(19) United States
(12) Patent Application Publication
(10) Pub. No.: US 2023/0226259 A1
(43) Pub. Date: Jul. 20, 2023

(54) NANOFIBER CARDIAC PATCH AND METHODS OF USE THEREOF

(71) Applicant: Ohio State Innovation Foundation, Columbus, OH (US)

(72) Inventors: Mahmood KHAN, Dublin, OH (US); Heather POWELL, Mason, OH (US)

(21) Appl. No.: 18/007,918

(22) PCT Filed: Jun. 3, 2021
(86) PCT No.: PCT/US2021/035676

§ 371 Date: Dec. 2, 2022

Related U.S. Application Data
(60) Provisional application No. 63/033,885, filed on Jun. 3, 2020.
(60) Provisional application No. 63/040,747, filed on Jun. 18, 2020.

(51) Int. Cl. A61L 27/48; A61L 27/54

(52) U.S. Cl. CPC A61L 27/48; A61L 27/54; A61L 2430/20; A61L 2400/12; A61L 2300/414

(57) ABSTRACT

The present disclosure relates to a biocompatible patch and methods of use thereof. A biocompatible patch and uses thereof for treating a damaged cardiac tissue.

### CROSS REFERENCE TO RELATED APPLICATIONS

This application claims the benefit of U.S. Provisional Patent Application Ser. No. 63/033,885, filed Jun. 3, 2020 and U.S. Provisional Patent Application Ser. No. 63/040,747 filed Jun. 18, 2020, which are expressly incorporated herein by reference.

### STATEMENT REGARDING FEDERALLY SPONSORED RESEARCH

This invention was made with government support under Grant No. R01HL136232 awarded by the National Institutes of Health. The Government has certain rights in the invention.

### FIELD

The present disclosure relates to nanofiber cardiac patches and uses thereof.

### BACKGROUND

Cardiovascular diseases (CVDs) are the leading cause of mortality worldwide. In the United States alone, an estimated 48% of adults suffer from some form of CVD. Cell-based approaches using adult stem/progenitor cells have been used for the treatment of CVDs in a number of pre-clinical and clinical studies. Unfortunately, the use of these stem cells has been challenging on account of their limited availability and low differentiation to functional cardiomyocytes post-transplantation. What is needed are compositions and methods for cell-based therapies for CVDs. The compositions and methods disclosed herein address these and other needs.

### SUMMARY

Disclosed herein is a biocompatible patch and uses thereof for treating a damaged cardiac tissue. In some aspects, disclosed herein is a biocompatible patch comprising:
a scaffold comprising a plurality of coaxial nanofibers, wherein the nanofibers comprise a polymeric core and a biocompatible shell; and
a cell, a tissue, or an organ in contact with a surface of the scaffold.

In some embodiments, the polymeric core comprises a material selected from the group consisting of polycaprolactone (PCL), poly(lactic-co-glycolic acid) (PLGA), polylactic acid (PLA), polyglycolide (PGA), and polyurethane (PU). In some embodiments, the biocompatible shell comprises a material selected from the group consisting of gelatin, collagen, collagen type I, collagen type IV, Matrigel, elastin, silk, laminin, and polyvinyl alcohol. In some embodiments, the plurality of nanofibers are aligned.

In some embodiments, the coaxial nanofibers have a diameter between about 200 nm to about 1000 nm.

In some embodiments, the biocompatible patch has a tensile strength between about 0.5 MPa to about 3.0 MPa.

In some embodiments, the biocompatible patch of any preceding aspect further comprises a growth factor. In some embodiments, the growth factor is incorporated into the biocompatible shell or on the surface of the biocompatible shell. In some embodiments, the growth factor is selected from the group consisting of vascular endothelial growth factor (VEGF), basic fibroblast growth factor (bFGF), insulin-like growth factor (IGF), placental growth factor (PIGF), angiopoietin-1, platelet derived growth factor-BB (PDGF-BB), and transforming growth factor β (TGF-β).

In some embodiments, the biocompatible patch of any preceding aspect further comprises fibronectin on the surface of the biocompatible shell.

In some embodiments, the cell comprises a stem cell or a cardiac cell. In some embodiments, the stem cell is selected from the group consisting of an induced pluripotent stem cell, a mesenchymal stem cell, and a cardiac progenitor cell.

In some embodiments, the tissue comprises a cardiac tissue.

In some embodiments, the patch is coated with polydopamine (PDA).

In some aspects, disclosed herein is a method for treating a damaged cardiac tissue in a subject, comprising transplanting the biocompatible patch of any preceding aspect to a site of the damaged cardiac tissue in the subject. In some embodiments, the method further comprises culturing the cell and the scaffold of the biocompatible patch of any preceding aspect ex vivo for at least 10 days prior to transplantation.

In some aspects, disclosed herein is a method of differentiating a stem cell, comprising:
contacting a stem cell with a surface of the scaffold of the biocompatible patch of any preceding aspect; and
culturing the stem cell.

In some embodiments, the stem cell is selected from the group consisting of an induced pluripotent stem cell, a mesenchymal stem cell, and a cardiac progenitor cell.

### BRIEF DESCRIPTION OF THE DRAWINGS

The accompanying figures, which are incorporated in and constitute a part of this specification, illustrate several aspects described below.

FIGS. 1A-1C show characterization of the electrospun scaffold. FIGS. 1A-1B show SEM images of gelatin, PCL and PCL-gelatin co-axial scaffolds. FIG. 1C shows ATR-FTIR analyses of gelatin, PCL, and co-axial nanofibrous scaffolds.

FIGS. 2A-2C show comparative assessment of hiPSCs cultured in 2D and on 3D PCL-gelatin co-axial scaffolds. FIG. 2A shows SEM images of hiPSCs cultured in 2D (I-II) and 3D (III-IV) cultures. Scale: I: 50 μm; II, IV: 10 μm; III: 500 μm. FIG. 2B shows fluorescent images showing the expression of PSC markers, OCT4 and SSEA4, in the human iPSCs cultures in 2D (I-IV) and 3D (V-VIII). Scale: 50 μm. FIG. 2C shows staining for viability of hiPSCs cultured in 3D. Representative fluorescence images showing the live (Calcein-AM, green) and dead (Propidium iodide, red) cells in 3D cultures. Scale: 375 μm.

FIGS. 3A-3E show morphological and functional assessment of cardiac differentiation of human iPSCs in 2D and 3D cultures. FIGS. 3A-3C show phase contrast image of human iPSCs differentiated to cardiomyocytes in 2D cultures on D14 (FIG. 3A) and D28 (FIG. 3B), and 3D cultures on D28 (FIG. 3C). Scale: 500 μm. FIG. 3D shows quantitative assessment of beating frequency of the hiPSCs differentiated to functional cardiomyocytes in 2D culture (grey) and 3D cultures (red). FIG. 3E depicts SEM images showing the surface morphology of the cells differentiated from hiPSCs cultured and differentiated to functional CMs in 2D (I-II, V-VI) and 3D (III-IV, VII-VIII) cultures on D14 and D28. Scale: I-IV: 50 μm; V, VIII: 10 μm; VI: 30 μm; and VII: 5 μm.

FIGS. 4A-4B show immunofluorescence analysis of cardiac differentiation of hiPSCs in 2D and 3D cultures. FIG. 4A shows confocal images showing the expression of cardiac progenitor marker NKX-2.5 on D7 during cardiac differentiation of hPSCs in 2D (I-III) and 3D (IV-VI). Scale: I-III: 50 μm: IV-VI: 20 μm. FIG. 4B shows immunofluorescence analysis of cardiomyocytes differentiated from hiPSCs in 2D and 3D cultures. Confocal images showing the expression of CM marker, sarcomeric alpha actinin (α-SA) in hiPSCs differentiated in 2D (I-III) and 3D (IV-VII). Dotted yellow lines represent the co-axial scaffold. Red and white arrows indicate fibers and cells, respectively. Scale: 50 μm.

FIGS. 5A-5D show gene expression analysis of cardiac progenitors (CP) and cardiomyocytes markers during cardiac differentiation of hiPSCs in 2D and 3D cultures. The bar graph shows the fold-change in expression of the CP-associated genes, SIRPA (FIG. 5A) and ISL1 (FIG. 5B), and CM-associated genes, MHC6 (FIG. 5C) and TNNT2 (FIG. 5D), in 2D and 3D cultures on DO, D7 and D28 of cardiac differentiation with respect to the DO (undifferentiated hiPSCs) samples. Values are represented as mean±SEM from 3 biological replicates. D represent days; ***p<0.001.

FIG. 6 shows schematic representation of cardiac scaffold transplantation onto the Fontan heart.

FIGS. 7A-7C show nanofiber scaffold with hCPCs. FIG. 7A shows SEM image of the aligned co-axial PCL-gelatin nanofiber scaffold. Inset shows the confocal image of the coaxial nanofibers, which clearly differentiates the core-shell structure (PCL, red; gelatin, green). FIG. 7B shows scaffold prepared for cell culture. FIG. 7C shows hCPCs grown on scaffold, stained with calcein-AM (green) for imaging live cells.

FIGS. 8A-8B show nanofiber scaffold with hiCMs. (FIG. 8A) SEM and (FIG. 8B) confocal image of the hiCMs seeded on a co-axial PCL-gelatin nanofiber scaffold.

FIGS. 9A-9E show functional assessment of hiPSC-CMs cultured in 2-D and 3-D cultures in response to Isoproterenol (ISO). Heat map showing the beat rate in representative wells of 2-D and 3-D cultures before treatment (baseline) and after treatment with ISO at different concentrations (FIG. 9A). Representative images showing changes in field potential in 2-D (FIG. 9B) and 3-D (FIG. 9C) cultures after treatment with ISO. Representative images showing changes in the number of beats before (baseline) and after treatment with 100 nM ISO (FIG. 9D). Quantitative assessment of fold change in FPDc following ISO treatment in 2-D and 3-D cultures (FIG. 9E). Data shown in (FIG. 9E) is mean±SD, n=8, from 3 independent cultures. ***: p<0.001 Vs the corresponding baseline; $p<0.001, #p<0.01 Vs corresponding 2-D cultures.

FIGS. 10A-10C show assessment of cardiac fibrosis and in vivo engraftment of Nanofiber cardiac patch at 4 weeks after transplantation post-MI. FIG. 10A shows isolated whole heart showing engraftment of cardiac patch onto the rat heart; FIG. 10B depicts transverse section of the heart showing strong integration of cardiac patch onto the epicardial heart surface; FIG. 10C shows Masson-trichrome staining indicating fibrosis (blue) and red area (white arrows) above the fibrotic scar indicating engraftment of transplanted hiCMs cardiac patch and survival of cardiomyocytes within the patch.

FIGS. 11A-11C show tracking of engrafted cardiac patch by magnetic resonance imaging (MRI) and assessment of angiogenesis in the patch in vivo at 4 weeks post-transplantation after MI. FIG. 11A depicts in vivo MRI imaging that shows hypo-intense regions (Yellow arrows) in the left ventricular wall, indicating presence of SPIO-labeled cardiomyocytes within the cardiac patch. FIG. 11B depicts immunostaining of cardiac section that shows fluorescent labeled dragon green SPIO particles in the heart along with cardiac troponin-T staining (red); FIG. 11C shows immunostaining with alpha smooth muscle actin, a marker for blood vessels (red), demonstrating the presence of blood vessels (White arrows) inside the cardiac patch indicating the cardiac patch promotes angiogenesis in vivo.

FIGS. 12A-12D show schematic of the aligned coaxial nanofibrous cardiac patch fabrication process and representative images of nanofibers. FIG. 12A shows that polycaprolactone (PCL) (8% w/v) and Gelatin (12% w/v) were employed for the fabrication of aligned coaxial (CoA) nanofibrous patches by an electrospinning method. FIG. 12B shows SEM image of the aligned CoA nanofibrous patch. Scale: 10 μM. FIG. 12C shows SEM image of a single CoA nanofiber showing core-shell structure. Scale: 4 μM. FIG. 12D shows confocal image of aligned CoA nanofibers showing the presence of PCL (red) in the core and gelatin (green) in the shell. Scale: 5 μM.

FIGS. 13A-13E show mechanical testing of aligned nanofibrous patches and comparison with aligned coaxial patch. FIG. 13A shows SEM images of aligned PCL, aligned gelatin, and aligned coaxial (CoA) nanofibrous patch. FIG. 13B shows FTIR spectra of aligned PCL, aligned gelatin, and aligned CoA nanofibrous patch. (FIG. 13C) Tensile strength, (FIG. 13D) Young's modulus, and (FIG. 13E) Stress vs elongation percentage for aligned PCL, aligned gelatin, and aligned CoA patches.

FIGS. 14A-14D show morphological analysis of hiPSC-CMs cultured on aligned coaxial patch. FIGS. 14A-14B show SEM images of hiPSC-CMs cultured on aligned coaxial (CoA) nanofibrous patches. Scale: FIG. 14A. 300 μM; FIG. 14B: 30 μM. FIG. 14C shows fluorescence image showing Calcein-AM staining of hiPSC-CMs cultured on CoA patches. FIG. 14D shows fold change in LDH released by hiPSC-CMs cultured in tissue culture plates (2-D) and CoA patches (3-D) as compared control. Data expressed as mean±SD, n=3.

FIGS. 15A-15B show expression of cardiac markers in cardiomyocytes cultured on the aligned coaxial patch. FIG. 15A shows confocal images showing the expression of α-SA, GATA4, cardiac TnT, and CX-43 in hiPSC-CMs cultured on aligned coaxial patches. FIG. 15B shows cross-section of aligned CoA patch showing the expression of α-SA, cardiac TnT, GATA4, and NKX 2.5 in the hiPSC-CMs.

FIGS. 16A-16G show calcium cycling and optical contractility analysis of the aligned coaxial cardiac patch. FIG. 16A shows representative images of calcium transients in 2-D and 3-D cultures. FIGS. 16B-16D show representative data of untreated control aligned coaxial (CoA) cardiac patch, and FIGS. 16E-16G show aligned CoA cardiac patch treated with 100 nM Isoproterenol (ISO), FIGS. 16B and 16E show spontaneous beat patterns expressed as displacement relative to a resting reference state vs time. FIG. 16C and FIG. 16F show fourier power spectra of beat patterns showing the dominant beat frequency as a peak and spatially resolved contractility analysis. FIGS. 16D and 16G show representation of PIV with warmer colors corresponding to higher divergence, a numerical estimate of contractile strength.

FIGS. 17A-17I show functional assessment of hiPSC-CMs cultured in 2-D and 3-D cultures in response to ISO and Verapamil. FIG. 17A depicts heat maps showing the beat rate in representative wells of 2-D and 3-D cultures before treatment (baseline) and after treatment with ISO or Verapamil at different concentrations. Representative images showing changes in beat detection before (baseline) and after treatment with (FIG. 17B) 100 nM ISO and (FIG. 17C) 0.3 μM Verapamil. Representative images showing changes in field potential in (FIGS. 17D and 17G) 2-D and (FIGS. 17E and 17H) 3-D cultures after treatment with (FIGS. 17D and 17E) ISO and (FIGS. 17G and 17H) Verapamil Quantitative assessment of change in FPDc and beat period after treatment with (FIG. 17F) ISO and (FIG. 17I) Verapamil, respectively, in 2-D and 3-D cultures. Dotted arrows in FIGS. 17D, 17E, 17G and 17H show FPDc. Data shown in FIGS. 17F, 17I is mean SD, n=8, from 3 independent cultures. **: p<0.01, ***: p<0.001 vs corresponding baseline; $:p<0.001, #:p<0.01 vs corresponding 2-D cultures.

FIGS. 18A-18G show functional assessment of hiPSC-CMs cultured in 2-D and 3-D cultures after treatment with E-4031. FIG. 18A depicts heat maps showing the beat rate in representative wells of 2-D and 3-D cultures before treatment (baseline) and after treatment with E4031 at different concentrations. FIG. 18B depicts representative images showing changes in beat detection before (baseline) and after treatment with E4031. FIG. 18C shows quantitative assessment of fold change in FPDc following E4031 treatment in 2-D and 3-D cultures. Representative images showing changes in field potential in (FIG. 18D) 2-D and (FIG. 18E) 3-D cultures after treatment with E4031. Quantitative assessment of change in (FIG. 18F) spike amplitude and (FIG. 18G) arrhythmicity after E-4031 treatment in 2-D and 3-D cultures. Dotted arrows in FIGS. 18D and 18E show FPDc. Data shown in FIGS. 18C, 18F, 18G is mean±SD, n=8, from 3 independent cultures. * p<0.05***: p<0.001 vs corresponding baseline; #: p<0.01 vs corresponding 2-D cultures.

FIG. 19 shows electrospinning set-up for fabrication of aligned co-axial (Co-A) PCL-gelatin nanofibrous scaffold. The 8% PCL solution (red) and 12% gelatin (green) were pumped at 1 ml/hr and 4 ml/hr, respectively through the co-axial nozzle such that gelatin flows through the outer nozzle while PCL flows through the inner nozzle. A voltage of 20 kV is applied to the nozzle tip. The extruded nanofibers have an outer shell of gelatin and inner core made of PCL. The fibers are collected onto a rotating collector to obtain aligned Co-A nanofibrous scaffolds.

FIG. 20 shows scanning electron microscopy (SEM) of co-axial aligned nanofibers: SEM image shows aligned (parallel) nanofibers within the scaffold. Scale bar: 20 μm.

FIGS. 21A-21D show crosslinking, coating and seeding hiPSC-CMs on the aligned nanofibrous scaffold. FIG. 21A shows that the aligned nanofibrous scaffold obtained by electrospinning is cut into 8 mm scaffold using a biopsy punch and cross-linked using 7 mM EDC solution. The cross-linked scaffold is sterilized by washing with 70% ethanol followed by PBS. FIG. 21B shows that, for coating, the cross-linked scaffold is placed on N-Terfaces and kept in 94 mm dishes containing drops of PBS (to prevent evaporation). 30 μl of 50 μg/ml fibronectin is added onto the scaffold and incubated for 1 hr at 37° C. FIG. 21C shows that, for seeding hiPSC-CMs onto the scaffold, a sterile sponge is placed in a 94 mm dish and sequentially washed with PBS and cardiomyocyte maintenance medium (CMM). The fibronectin-coated scaffold along with the N-Terface are placed on the inoculation sponge and cell suspension is added dropwise onto the scaffold. The dish is incubated for 2-4 hrs at 37° C., 5% CO₂. FIG. 21D shows that the scaffold along with the N-Terface is placed in a 6-well plate containing CMM and incubated overnight. The following day, the N-Terface is removed and scaffolds are left freely floating in CMM.

FIG. 22 shows Scanning Electron Microscope imaging of hiPSC-CMs cultured on aligned nanofibers: SEM image shows stoichiometric alignment of hiPSC-CMs in the direction of the nanofibers within the cardiac scaffold. Scale bar: 50 μm.

FIG. 23 shows structure of SARS-CoV-2. The virus is made up of four proteins: membrane (M) protein, envelope (E) protein, nucleocapsid (N) protein and spike (S) protein. A single stranded RNA constitutes the viral genome.

FIGS. 24A-24B show number of clinical trials registered: FIG. 24A depicts a chart showing the number of clinical trials for COVID-19, hydroxychloroquine (HCQ), and cardiovascular diseases (CVDs). FIG. 24B shows global distribution of clinical trials using hydroxychloroquine for COVID-19. Source: clinicaltrials.gov/.

FIG. 25 shows schematic of experimental design.

FIGS. 26A-26C show nanofiber scaffold with hiCMs. FIG. 26A shows SEM image of the aligned co-axial PCL-gelatin nanofiber scaffold. FIG. 26B shows confocal image of the coaxial nanofibers, which clearly differentiates the core-shell structure (PCL, red; gelatin, green). FIG. 26C shows hiCMs grown on 3-D scaffold, calcein-AM (green) staining for live cells.

FIGS. 27A-27F show functional assessment of hiPSC-CMs cultured in 2-D and 3-D cultures in response to ISO and E4031. FIG. 27A depicts a heat map showing the beat rate in representative wells of 2-D and 3-D cultures before treatment (baseline) and after treatment with ISO or E4031 at different doses. FIG. 27B depicts representative images showing changes in the number of beats before (baseline) and after treatment with 100 nM ISO and E4031. FIG. 27C shows quantitative assessment of fold change in FPDc following ISO treatment in 2-D and 3-D cultures. FIG. 27D depicts representative images showing changes in field potential in 2-D and 3-D cultures after treatment with ISO and E4031. FIGS. 27E-27F show quantitative assessment of fold change in FPDc and arrhythmicity following E4031 treatment in 2-D and 3-D cultures. Data shown in FIGS. 27C, 27E-27F is mean±SD, n=8, from 3 independent cultures. *p<0.05 ***: p<0.001 Vs the corresponding baseline; $p<0.001, #:p<0.01 Vs corresponding 2-D cultures.

FIG. 28 shows morphological comparison of hiCMs and adult human heart tissue. TEM imaging showing similar sarcomere structure and organization of hiCMs and adult human heart tissue. Scale 500 nm.

FIGS. 29A-29B show cryo-TEM and SEM imaging of PDA-NPs and DPDA-NPs.

FIGS. 30A-30E show characterization of particles. FIG. 30A. UV-Vis Spectroscopy of PDA-NPs and DPDA-NPs, FIG. 30B. Zeta-potential analysis of PDA-NPs/DPDA-NPs entrapping bFGF. FIG. 30C. Nanoparticle tracking analysis of PDA NPs and DPDA NPs. FIG. 30D. Quantification of unentrapped bFGF when loaded in the ratio of 1:100 (bFGF:NPs). FIG. 30E. Release studies of bFGF from PDA-NPs and DPDA-NPs.

FIG. 31 shows antioxidant efficacy of PDA-NPs and DPDA-NPs at different time intervals (5 and 60 min post-incubation) compared to ascorbic acid.

FIGS. 32A-32B show biocompatibility of PDA-NPs and DPDA-NPs. FIG. 32A. Biocompatibility of PDA-NPs and DPDA-NPs assessed in HUVEC cell line at 48 h time point. FIG. 32B. Confocal image showing the Intracellular uptake of PDA-NPs (green) in hiPSC-CMs showing expression of cardiac troponin T (red).

FIGS. 33A-33G show modulation of calcium transients in hiPSC-CMs following treatment with PDA NPs. Changes in Fluo-4-AM fluorescence in Control (FIG. 33A) and 25 μg/mL PDA NP-treated hiPSC-CMs (FIG. 33B) after 15 min. Graphs showing fold change in amplitude (FIG. 33C), time to peak (FIG. 33D), transient duration (FIG. 33E) and decay time (FIG. 33F) of calcium transients in PDA NP-treated hiPSC-CMs versus control hiPSC-CMs. n=40 cells in each group from three independent experiments. FIG. 33G shows fold change in beat period with varying dose of PDA-NPs ***. p<0.001, ****: p<0.0001

FIGS. 34A-34B show new blood vessel invading the mesh. FIG. 34A. Schematic representing the experimental setup. FIG. 34B. Stereomicroscopic images of nylon mesh coated with geltrex containing test samples, arrows shows the sprouting of new blood vessels invading the mesh.

FIGS. 35A-35B show images of electrospun nanofiber. FIG. 35A. Microscopic images of electrospun nanofiber patch coated in-situ with polydopamine. FIG. 35B. PDA-NPspassive binding the electrospun nanofibers.

FIGS. 36A-36B show photothermal suturing of PDA coated patches. FIG. 36A. Ex-vivo photothermal suturing of PDA coated patches on isolated mouse tissue sample. FIG. 36B. NIR light mediated photothermal suturing of PDA coated patch on isolated mouse beating heart.

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

The terms “about” and “approximately” are defined as being “close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

“Activate”, “activating”, and “activation” mean to increase an activity, response, condition, or other biological parameter. This may also include, for example, a 10% increase in the activity, response, or condition, as compared to the native or control level. Thus, the increase can be a 10, 20, 30, 40, 50, 60, 70, 80, 90, 100%, or any amount of reduction in between as compared to native or control levels.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The term “biocompatible” generally refers to a material and any metabolites or degradation products thereof that are generally non-toxic to the recipient and do not cause significant adverse effects to the subject.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Decrease” can refer to any change that results in a lower level of gene expression, protein expression, amount of a symptom, disease (e.g., a cardiovascular disease), composition, condition, or activity. A substance is also understood to decrease the level of the gene, the protein, the composition, or the amount of the condition when the level of the gene, the protein, the composition, or the amount of the condition is less/lower relative to the output of the level of the gene, the protein, the composition, or the amount of the condition without the substance. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Increase” can refer to any change that results in a higher level of gene expression, protein expression, amount of a symptom, disease (e.g., a cardiovascular disease), composition, condition, or activity. A substance is also understood to increase the level of the gene, the protein, the composition, or the amount of the condition when the level of the gene, the protein, the composition, or the amount of the condition is more/higher relative to the output of the level of the gene, the protein, the composition, or the amount of the condition without the substance. Also, for example, an increase can be a change in the symptoms of a disorder such that the symptoms are less than previously observed. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

As used herein, “induce”, as well as the correlated term “induction”, refer to the action of generating, promoting, forming, regulating, activating, enhancing or accelerating a biological phenomenon.

The term “polymer” as used herein refers to a relatively high molecular weight organic compound, natural or synthetic, whose structure can be represented by a repeated small unit, the monomer. Synthetic polymers are typically formed by addition or condensation polymerization of monomers. The polymers used or produced in the present invention are biodegradable. The polymer is suitable for use in the body of a subject, i.e. is biologically inert and physiologically acceptable, non-toxic, and is biodegradable in the environment of use, i.e. can be resorbed by the body. The term “polymer” encompasses all forms of polymers including, but not limited to, natural polymers, synthetic polymers, homopolymers, heteropolymers or copolymers, addition polymers, etc.

The term “subject” refers to a human in need of treatment for any purpose, and more preferably a human in need of treatment to treat a disease or disorder. The term “subject” can also refer to non-human animals, such as dogs, cats, horses, cows, pigs, sheep and non-human primates, among others.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition refers to an amount that is effective to achieve a desired therapeutic result. In some embodiments, a desired therapeutic result is the control of a cardiovascular disease or a symptom thereof. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

### Biocompatible Patch and Methods of Use

In some aspects, disclosed herein is a biocompatible patch comprising:
a scaffold comprising a plurality of coaxial nanofibers, wherein the nanofibers comprise a polymeric core and a biocompatible shell; and
a cell, a tissue, or an organ in contact with a surface of the scaffold.

The term “nanofiber” is used herein to refer to materials that are in the form of continuous filaments or discrete elongated pieces of material, and that typically have diameters of less than or equal to 1000 nm. In this regard, the term “scaffold” is used herein to refer to the arrangement of such nanofibers into a supporting framework that can then be used to support cells or other additional materials.

The present disclosure is not limited to a particular polymer. Any degradable or non-degradable polymer can be utilized. Examples include, but are not limited to, polymers and copolymers of carboxylic acids such as glycolic acid and lactic acid, polyurethanes, polyesters such as poly(ethylene terephthalate), polyamides such as nylon, polyacrylonitriles, polyphosphazines, polylactones such as polycaprolactone, and polyanhydrides such as poly[bis(p-carboxphenoxy)propane anhydride] and other polymers or copolymers such as polyethylene, polyvinyl chloride and ethylene vinyl acetate, homopolymers and copolymers of delta-valerolactone, and p-dioxanone as well as their copolymers with caprolactone, and those described in U.S. Pat. No. 6,290,729, herein incorporated by reference in its entirety. In some embodiments, the polymeric core comprises a material selected from the group consisting of polycaprolactone, poly(lactic-co-glycolic acid), and polylactic acid. In some embodiments, the polymeric core comprises a material selected from polycaprolactone (PCL), poly(lactic-co-glycolic acid) (PLGA), polylactic acid (PLA), polyglycolide (PGA), and polyurethane (PU). In some embodiments, the polymeric core comprises PCL. In some embodiments, the polymeric core comprises PLGA. In some embodiments, the polymeric core comprises PLA. In some embodiments, the polymeric core comprises PGA. In some embodiments, the polymeric core comprises PU. In some embodiments, the polymeric core comprises a copolymer of two or more polymers disclosed herein.

In some embodiments, the biocompatible shell comprises a material selected from the group consisting of gelatin, collagen, collagen type I, collagen type IV, Matrigel, elastin, silk, laminin, and polyvinyl alcohol. In some embodiments, the biocompatible shell comprises gelatin. In some embodiments, the biocompatible shell comprises collagen type I. In some embodiments, the biocompatible shell comprises type IV. In some embodiments, the biocompatible shell comprises Matrigel. In some embodiments, the biocompatible shell comprises elastin. In some embodiment, the biocompatible shell comprises silk. In some embodiments, the biocompatible shell comprises polyvinyl alcohol. In some embodiments, the biocompatible shell comprises laminin. In some embodiments, the biocompatible shell comprises polydopamine (PDA).

It should be understood that Matrigel used herein is a gelatinous protein mixture secreted by Engelbreth-Holm-Swarm (EHS) mouse sarcoma cells produced by Corning Life Sciences. It is a solubilized basement membrane preparation extracted from the Engelbreth-Holm-Swarm (EHS) mouse sarcoma, a tumor rich in such ECM proteins as laminin (a major component), collagen IV, heparin sulfate proteoglycans, entactin/nidogen, and a number of growth factors.

In some embodiments, the scaffold is comprised of only biopolymers/proteins including gelatin, silk, collagen, collagen type 1, collagen type IV, laminin and/or any combination of these materials. Some embodiments require no crosslinking and some require physical crosslinking such as dehydrothermal crosslinking (i.e. heating to elevated temperatures under vacuum). Some require chemical crosslinking which can include exposure to glutaraldehyde vapor, glutaraldehyde solutions, ethylaminodipropyl carbodiimide solutions, and/or genipin solutions. Additionally, combinations of physical and chemical cross-linking strategies may be utilized.

Accordingly, in some aspects, disclosed herein is a biocompatible patch comprising a scaffold comprising a plurality of coaxial nanofibers, wherein the nanofibers comprise a polymeric core and a biocompatible shell, wherein the polymeric core comprises polycaprolactone and the biocompatible shell comprises gelatin.

In some embodiments, the ratio of polycaprolactone to gelatin in the nanofiber is about 6:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, or 1:6.

In some embodiments, the PCL core has a diameter between 200 nm to about 1000 nm, between 200 nm to about 900 nm, between 200 nm to about 800 nm, between 300 nm to about 800 nm, between about 400 nm to about 700 nm, at least 200 nm, at least 300 nm, at least 400 nm, at least 500 nm, at least 600 nm, at least 700 nm, or at least 800 nm. The PCL/gelatin nanofibers of embodiments of the present disclosure exhibit improved Young's modulus and tensile strength relative to gelatin and improved elongation relative to PCL and are suitable for cell, tissue, or organ culture. Thus, the nanofibers of embodiments of the present disclosure provide improved strength in a fiber for use as a scaffold for biological applications. In some embodiments, the biocompatible patch has a tensile strength between about 0.3 MPa to about 5.0 MPa, between about 0.5 MPa to about 3.0 MPa, or between 0.5 MPa to about 1.0 MPa.

The nanofibers disclosed herein are made using electrospinning. Electrospinning as a facile and universal fiber-forming technique has enabled the fabrication of a variety of biomaterials into micro/nanometer-diameter fibers, including synthetic polymers (Reneker, D. H. et al., Nanotechnology 1996, 7, 216-223; Li, D. et al., Adv Mater 2004, 16, 1151-1170), proteins (Li, C. M. et al., Biomaterials 2006, 27, 3115-3124; Li, M. Y. et al., Biomaterials 2005, 26, 5999-6008) and lipids (McKee, M. G. et al, Science 2006, 311, 353-355; Zha, Z. B. et al., Adv Mater 2011, 23, 3435-3440).

In some embodiments, the plurality of the nanofibers are aligned. In some embodiments, the plurality of the nanofibers are parallelly aligned.

In some embodiments, the biocompatible patch of any preceding aspect further comprises a growth factor. In some embodiments, the growth factor is incorporated into the biocompatible shell or on the surface of the biocompatible shell. In some embodiments, the growth factor is on the surface of the biocompatible shell. In some embodiments, the growth factor is selected from the group consisting of vascular endothelial growth factor (VEGF), basic fibroblast growth factor (bFGF), insulin-like growth factor (IGF), placental growth factor (PIGF), angiopoietin-1, platelet derived growth factor-BB (PDGF-BB), and transforming growth factor β (TGF-β).

In some embodiments, the mean thickness of the scaffold of the biocompatible patch disclosed herein is between about 50 μm and about 500 μm, between about 80 μm and about 400 μm, between about 80 μm and about 300 μm, between about 80 μm and about 250 μm, between about 80 μm and about 200 μm, between about 80 μm and about 180 μm, between about 90 μm and about 160 μm, between about 90 μm and about 150 μm, between about 90 μm and about 140 μm, or between about 90 μm and about 130 μm; at least 70 μm, at least 80 μm, at least 90 μm, at least 100 μm, at least 110 μm, at least 120 μm, at least 130 μm, at least 140 μm, at least 150 μm, at least 160 μm, at least 180 μm, at least 190 μm, at least 250 μm, at least 300 μm, at least 400 μm, at least 500 μm, at least 600 μm, at least 700 μm, or at least 800 μm.

In some embodiments, the biocompatible patch disclosed herein comprises a cell, a tissue, or an organ in contact with a surface of the scaffold.

In some embodiments, the tissue comprises a cardiac tissue, connective tissue, muscle tissue, nervous tissue, or epithelial tissue. In some embodiments, the tissue comprises a cardiac tissue.

In some embodiments, the cell comprises a stem cell, a cardiac cell, an islet cells, a fibroblast, a hormone secreting cells, a neural cell, an epithelial cell, or an endothelial cell. In some embodiments, the cell comprises a cardiac cell. In some embodiments, the cell comprises a stem cell or a cardiac cell. In some embodiments, the cell comprises a cardiomyocyte. In some embodiments, the cardiac cell is differentiated from a stem cell. In some embodiments, the stem cell is selected from the group consisting of an induced pluripotent stem cell, a mesenchymal stem cell, and a cardiac progenitor cell. In some embodiments, the stem cell comprises an induced pluripotent stem cell. In some embodiments, the stem cell comprises a mesenchymal stem cell. In some embodiments, the stem cell comprises a cardiac progenitor cell. In some embodiments, the cell is a human cell. In some embodiments, the cell is an engineered cell.

In some embodiments, the organ is selected from heart, stomach, liver, gallbladder, pancreas, intestines, colon, rectum, anus, hypothalamus, pituitary gland, pineal body or pineal gland, thyroid, parathyroid, adrenal, kidney, tonsils, adenoid, thymus, spleen, hair, brain, spinal cord, nerves, ovaries, fallopian tubes, uterus, vagina, mammary glands, testes, vas deferens, seminal vesicles, prostate, penis, pharynx, larynx, trachea, bronchi, lungs, diaphragm, bones, cartilage, ligaments, and tendons.

In some aspects, disclosed herein is a biocompatible patch comprising:
a scaffold comprising a plurality of coaxial nanofibers, wherein the nanofibers comprise a polymeric core and a biocompatible shell.

In some embodiments, the patch is coated with polydopamine (PDA). In some embodiments, the biocompatible patch of any preceding aspect further comprises a growth factor.

In some aspects, disclosed herein is a method for treating a damaged cardiac tissue in a subject, comprising transplanting the biocompatible patch of any preceding aspect to a site of the damaged cardiac tissue in the subject.

In some aspects, disclosed herein is a method for treating a cardiovascular disease in a subject, comprising transplanting the biocompatible patch of any preceding aspect to a site of the damaged cardiac tissue in the subject.

In some aspects, disclosed herein is a method for treating a damaged cardiac tissue in a subject, comprising transplanting the scaffold of the biocompatible patch of any preceding aspect to a site of the damaged cardiac tissue in the subject.

In some aspects, disclosed herein is a method for treating a damaged cardiac tissue in a subject, comprising transplanting a biocompatible patch to a site of the damaged cardiac tissue in the subject, wherein the biocompatible patch comprises:
a scaffold comprising a plurality of coaxial nanofibers, wherein the nanofibers comprise a polymeric core and a biocompatible shell; and
a cell, a tissue, or an organ in contact with a surface of the scaffold.

In some embodiments, the subject is a human. In some embodiments, the subject has a cardiovascular disease. In some embodiments, the subject has ischemic heart disease. In some embodiments, the subject has previously received Fontan procedure. In some embodiments, the subject has a congenital heart disorder.

In some embodiments, the cell, the tissue, or the organ is derived from the subject. In some embodiments, the cell, the tissue, or the organ is not derived from the subject. In some embodiments, the cell, the tissue, or the organ has been engineered.

In some embodiments, the method of any preceding aspect further comprises culturing the cell and the scaffold of the biocompatible patch ex vivo for at least 4 days (e.g., at least 5 days, at least 6 days, at least 7 days, at least 8 days, at least 9 days, at least 10 days, at least 11 days, at least 12 days, at least 13 days, at least 14 days, at least 15 days, at least 16 days, at least 18 days, at least 20 days, at least 22 days, at least 24 days, at least 26 days, at least 28 days, at least 30 days, at least 35 days, at least 40 days, at least 45 days, at least 50 days, at least 55 days, or at least 60 days) prior to transplantation.

It should be understood therein that the method disclosed herein increases cardiac cell survival and migration to the biocompatible patch. In some embodiments, the method disclosed herein improves the angiogenesis and proliferation of cardiac cells and/or tissues.

In some aspects, disclosed herein is a method of differentiating a stem cell, comprising: contacting a stem cell with a surface of the scaffold of the biocompatible patch of any preceding aspect; and culturing the stem cell.

In some embodiments, the stem cell is selected from the group consisting of an induced pluripotent stem cell, a mesenchymal stem cell, and a cardiac progenitor cell.

In some aspects, disclosed herein is a method for assessing cardiotoxicity of a drug, comprising:
a) contacting a stem cell with a surface of the scaffold of the biocompatible patch of any preceding aspect;
b) providing a culture condition that differentiates the stem cell to a cardiac cell; and
c) administering the drug to the differentiated cardiac cell of step b).

In some embodiments, differentiation of the stem cell takes at least at least 5 days, at least 6 days, at least 7 days, at least 8 days, at least 9 days, at least 10 days, at least 11 days, at least 12 days, at least 13 days, at least 14 days, at least 15 days, at least 16 days, at least 18 days, at least 20 days, at least 22 days, at least 24 days, at least 26 days, at least 28 days, at least 30 days, at least 35 days, at least 40 days, at least 45 days, at least 50 days, at least 55 days, or at least 60 days.

In some embodiments, the drug is hydroxychloroquine.

In some embodiments, the cardiac cell comprises a cardiomyocyte. In some embodiments, the cardiac cell is derived from a healthy human or a dilated cardiomyopathy patient. In some embodiments, the method for assessing cardiotoxicity of any preceding aspect further comprises transducing the differentiated cardiac cell with a pseudotyped virus comprising a SARS-CoV-2 protein. In some embodiments, the SARS-CoV-2 protein is SARS-CoV-2 spike protein, nucleocapsid protein, envelope protein, and/or membrane protein or a fragment thereof. In some embodiments, the SERS-CoV-2 protein is SARS-CoV-2 spike protein or a fragment thereof. In some embodiments, the pseudotyped virus is a pseudotyped murine leukemia virus (MLV).

In some aspects, disclosed herein is a method for treating a damaged cardiac tissue in a subject, comprising;

coating the biocompatible patch of any preceding aspect with polydopamine (PDA);

transplanting the PDA-coated biocompatible patch to a site of the damaged cardiac tissue in the subject; and

irradiating the PDA-coated biocompatible patch with near-infrared light to attach the PDA-coated biocompatible patch to the cardiac tissue in the subject. In some embodiments, the biocompatible patch of any preceding aspect further comprises a growth factor.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. In Situ Differentiation of Human-Induced Pluripotent Stem Cells into Functional Cardiomyocytes on a Coaxial PCL-Gelatin Nanofibrous Scaffold

Human induced pluripotent stem cells (hiPSCs)-derived cardiomyocytes (hiPSC-CMs) have been explored for cardiac regeneration and repair as well as for development of in vitro 3D cardiac tissue models. Existing protocols for cardiac differentiation of hiPSCs utilize a 2D culture system. However, the efficiency of hiPSC differentiation to cardiomyocytes in 3D culture systems has not been extensively explored. The present study investigated the efficiency of cardiac differentiation of hiPSCs to functional cardiomyocytes on 3D nanofibrous scaffolds. Co-axial polycaprolactone (PCL)-gelatin fibrous scaffolds were fabricated by electrospinning and characterized using SEM and FTIR spectroscopy. hiPSCs were cultured and differentiated into functional cardiomyocytes on the nanofibrous scaffold and compared with 2D cultures. To assess the relative efficiencies of both the systems, scanning electron microscopy (SEM), immunofluorescence and gene expression analyses were performed. Contractions of differentiated cardiomyocytes were observed in 2D cultures at 2-weeks and in 3D cultures at 4-weeks. SEM analysis showed no significant differences in the morphology of the cells differentiated on 2D versus 3D cultures. However, gene expression data showed significantly increased expression of cardiac progenitor genes (ISL-1, SIRPA) in 3D cultures and cardiomyocytes markers (TNNT, MHC6) in 2D cultures. In contrast, immunofluorescence staining showed no substantial differences in the expression of NKX-2.5 and α-sarcomeric actinin. Furthermore, uniform migration and distribution of the in situ differentiated cardiomyocytes was observed in the 3D fibrous scaffold. Overall, this study demonstrates that co-axial PCL-gelatin nanofibrous scaffolds can be used as a 3D culture platform for efficient differentiation of hiPSCs to functional cardiomyocytes.

### 1. Introduction

Cardiovascular diseases (CVDs) are the leading cause of mortality worldwide. In the United States alone, an estimated 48% of adults suffer from some form of CVD. Cell-based approaches using adult stem/progenitor cells have been used for the treatment of CVDs in a number of pre-clinical and clinical studies. Unfortunately, the use of these stem cells has been challenging on account of their limited availability and low differentiation to functional cardiomyocytes post-transplantation. In contrast, since their discovery, human induced pluripotent stem cells (hiPSCs), have overcome this limitation and hence, opened up new avenues in the field of cardiovascular regenerative medicine. Moreover, cardiomyocytes differentiated from hiPSCs (hiPSC-CMs) have become increasingly popular for use as in vitro model systems for cardiac developmental biology studies, disease modeling, and toxicology studies, as well as for development of a new generation cell-based therapies for CVDs.

Indeed, to conduct these studies, many protocols have been standardized for the efficient differentiation of hiPSCs to functional cardiomyocytes. However, the majority of these studies have been conducted using 2-dimensional (2D) culture systems. Moreover, recent reports, have clearly shown that hiPSC-CMs differentiated in 2D cultures do not completely resemble adult cardiomyocytes. Similarly, 3D cultures can represent a better model system to recapitulate in vivo differentiation of hiPSCs. Among different 3D culture systems, enhanced differentiation of hiPSCs, to chondrogenic cells, definitive endoderm cells, pancreatic β-cells, and neuronal cells, when cultured on 3-dimensional (3D) scaffolds. Adipose-derived stem cells and mouse pluripotent stem cells have also been shown to have improved differentiation to functional cardiomyocytes on 3D scaffolds. A detailed study investigating the efficiency of cardiac differentiation of hiPSCs on 3D scaffolds is currently lacking.

Synthetic polymers like polycaprolactone (PCL), poly (glycerol sebacate) (PGS), poly(lactic-co-glycolic acid) (PLGA) and poly (lactic acid) (PLA) have been commonly used for synthesis of nanofibrous scaffolds for culture and differentiation of hiPSCs. Although studies have reported cardiac differentiation of hiPSCs on these scaffolds, some studies have reported a negative effect of these polymers on cell viability and function due to the inherent hydrophobicity and poor biocompatibility of the polymer, as well as lack of cell adhesion sites. Biopolymers like fibrin, collagen, and gelatin have been commonly used as a substrate for culturing hiPSCs in 2D, mainly due to their biomimetic properties. However, their use in fabrication of 3D scaffolds has been limited as a result of their poor mechanical strength and degradation in prolonged culture. Hence, the fabrication of a stable, nanofibrous scaffold with superior biomimetic and mechanical properties is important for an understanding the differentiation of hiPSCs to functional cardiomyocytes in 3D cultures.

In this example, a co-axial PCL-gelatin nanofibrous scaffold was fabricated and characterized with a gelatin shell and a PCL core. Using this nanofibrous scaffold, a protocol was established for culturing and differentiation of hiPSCs into functional cardiomyocytes in a 3D microenvironment. Additionally, the cardiac differentiation of hiPSCs was compared in 3D and 2D cultures, to understand the relative efficiencies of differentiation and maturation of hiPSC-CMs in vitro in the two culture systems.

### 2. Materials and Methods

### 2.1. Preparation of Polycaprolactone (PCL)-Gelatin Co-Axial Nanofibrous Scaffold

PCL-gelatin coaxial randomly aligned fibrous scaffolds were fabricated using electrospinning. Gelatin (12% w/v) and PCL (8%) were dissolved in 1,1,1,3,3,3-hexafluoro-2-propanol (HFIP, Sigma-Aldrich, MA). The PCL solution was fed to the inner tube of the coaxial spinneret with the flow-rate of 1 ml/hr, while the gelatin solution was fed to the outer needle at a flow-rate of 4 ml/hr. The distance between the spinneret tip and the grounded rotating collector was kept at 20 cm and the voltage applied at the tip was 20 kV. Scaffolds were thoroughly dried and cut into circular patches of 8- or 12-mm diameter prior to crosslinking in a 7 mM 1-ethyl-3-(3-dimethylaminopropyl)carbodiimide hydrochloride (EDC, Sigma-Aldrich, MO) solution in 100% ethanol overnight to stabilize the gelatin shell. The patches were then disinfected by soaking in 70% ethanol for 24 hours, followed by three rinses in sterile PBS and one rinse in Essential 8™ culture medium.

2.2. Culturing of hiPSCs on a 3D Scaffold

Maintaining hiPSCs in 2D cultures: The hiPSC line (SCVI840), re-programmed from peripheral blood mononuclear cells (PBMCs), used in the study was obtained from the Stanford Cardiovascular Institute (SCVI) Biobank and the Stem Cell Core Facility of Genetics, Stanford University. The hiPSCs were cultured and maintained as previously described. Briefly, the hiPSCs were thawed and cultured in Essential 8™ Medium (E8, Thermo Fisher Scientific, MA) on Vitronectin XF (Stem Cell Technologies, Canada)-coated 6-well plates (Greiner, NC). The medium was supplemented with 10 μM of Rho-associated protein kinase (ROCK) inhibitor (Y-27632, TOCRIS, MN) for the first 24 hours of culture. Once the cultures attained a confluence of >80% in the dish, the cells were passaged onto Matrigel® (354277, Corning, N.Y.) coated 12-well plates for 2D culture or onto the PCL-gelatin scaffolds for 3D cultures.

Preparing the 3D scaffold for the plating of hiPSCs: PCL-gelatin scaffolds were coated with Matrigel® (Cat. No. 354277, Corning, N.Y.) for 1-2 hours. The Matrigel®-coated patches were then placed on top of a sterile N-Terface® (Winfield Labs, TX), which was further placed on a sterile surgical sponge (Hydrosorb: Carwil Corp, New London, Conn.) pre-soaked in E8 medium supplemented with 10 μM ROCK inhibitor in 94-mm dishes. Human iPSCs cultured in 2D culture plates were dissociated into single cells by incubating with Gentle Cell Dissociation Reagent (Stem Cell Technologies, Canada) in E8 medium supplemented with 10 μM ROCK inhibitor to get a concentrated cell suspension containing of 0.2 million cells/100 μl, which was then added dropwise onto individual 3D patches (0.2 million cells per 12 mm patch). The seeded patches were incubated for 7-8 hrs to ensure cell attachment then transferred to a 12-well plate containing E8 medium, with one patch per well.

2.3. Cardiac Differentiation of hiPSCs

Cardiac differentiation of hiPSCs was performed using a Cardiomyocyte Differentiation Kit (Thermo Fisher Scientific, MA), as per the manufacturer's instructions. Briefly, on Day 0 (DO) of differentiation, the hiPSC colonies, with 50-60% confluence, were incubated with Cardiomyocyte Differentiation Medium A for 48 hrs. On Day 2 (D2), the medium was replaced with Cardiomyocyte Differentiation Medium B for another 48 hrs. From Day 4 (D4) onwards, the cells were cultured in Cardiomyocyte Maintenance Medium. The medium for the cultures was changed every other day. The morphological changes during cardiac differentiation of hiPSCs were assessed by phase-contrast imaging using a Leica DM IL LED microscope (Leica Microsystems, Germany). Videos were recorded to monitor the contractility of functional cardiomyocytes and quantitative analysis was performed post-acquisition. For quantitative assessment of the beating frequencies of differentiated cardiomyocytes, the beats per minute were manually counted at different days of differentiation in four patches or wells from three independent experiments.

### 2.4. Scanning Electron Microscopy (SEM) Analysis

To assess the morphology, hiPSCs were cultured and differentiated on glass coverslips or electrospun scaffolds and processed as previously described. Briefly, the cells were fixed in 4% paraformaldehyde (PFA) for 24 hours at 4° C. and dehydrated sequentially in 50%, 70%, 85%, 90% and 100% ethanol gradients. The samples were finally dried using a graded series of hexamethyldisilazane (HMDS, Sigma-Aldrich, MO) in ethanol. After processing, the patches were affixed to SEM stubs using carbon tape and sputter coated with gold-palladium (Pelco Model 3). For characterization of the acellular scaffolds, scaffolds were cut, affixed to SEM stubs and sputter coated with gold-palladium coating. All samples were imaged on the Nova NanoSEM 400 microscope (FEI, OR) at 5 kEV.

### 2.5. Fourier-Transform Infrared Spectroscopy (FTIR) Studies

Surface chemical analysis of the patches was assessed using attenuated total reflection Fourier transform infrared spectroscopy (ATR-FTIR) in the range of 400-4000 cm⁻¹ (Thermo Nicolet Nexus 670 FTIR spectrometer, MN). Approximately 25-40 scans were performed on gelatin, PCL and PCL-gelatin coaxial scaffolds.

### 2.6. Immunofluorescence Studies

Immunofluorescence imaging was performed on undifferentiated and differentiated cells cultured on 3D patches and on coverslips (2D). The cultured cells were washed twice with DPBS and processed for immunostaining as described previously. Briefly, the cells were fixed in 4% PFA for 15 minutes at RT, permeabilized using 0.2% triton X-100 and incubated in blocking buffer containing 1% bovine serum albumin (BSA, Sigma-Aldrich, MO). The cells were incubated with the primary antibody overnight at 4° C. followed by the secondary antibody for 1 hour at RT in dark. The cells were counterstained with DAPI (Thermo Fisher Scientific, MA) and the coverslips were mounted over glass slides using ProLong™ Gold Antifade Mountant (Life Technologies, MA). The cells were then imaged on the Olympus FV3000 (Olympus Life Sciences, PA) confocal microscope. For cells differentiated in 3D, the patches were fixed in 4% paraformaldehyde, embedded in Optimal Cutting Temperature (OCT) compound, and sectioned at 7 μm using the Leica CM 1950 cryostat (Leica Biosystems, Germany). The sections were mounted onto glass slides and immunostaining was performed as described above. The antibodies used for immunostaining are as follows: rabbit anti-Oct4 (1:200, Thermo Fisher Scientific, MA, Cat #PA5-27438), mouse anti-SSEA4 (1:200, Thermo Fisher Scientific, MA, Cat #MA1-021), rabbit anti-Troponin T (1:200, Sigma-Aldrich, MO, Cat #HPA017888), mouse anti-Sarcomeric Alpha-Actin (1:200, Sigma-Aldrich, MO, Cat #A7811), rabbit anti-NKX-2.5 (1:200, Thermo Fisher Scientific, MA, Cat #PA5-49431), anti-mouse Alexa Fluor® 488 (1:1000, Cell Signaling Technology, MA, Cat #4408S), anti-rabbit Alexa Fluor® 594 (1:1000, Cell Signaling Technology, MA, Cat #8889S).

### 2.7. Gene Expression Analysis

At culture days 0, 7 and 28, cellular gene expression profile was analyzed. The cells were lysed in TRIzol (Invitrogen. MA) and total RNA was isolated using the Direct-zol RNA Miniprep kit (Zymo Research, CA) as per the manufacturer's instructions. The quantity and purity of the RNA was assessed using a NanoDrop200 spectrophotometer (Thermo Fisher Scientific, MA). First strand cDNA was synthesized using the RT² First Strand Kit (330404, Qiagen, MD) as per the manufacturer's instructions with the RT reaction being performed for 1 hour at 37° C. qRT-PCR was performed using Qiagen RT² SYBR Green ROX qPCR Mastermix (330523, Qiagen, MD) according to the manufacturer's protocol. The reaction was performed on a QuantStudio 3 (Applied Biosystems, MA) using QuantStudio design and analysis software V.1.4.1. Gene expression was normalized to the geometric mean of three housekeeping genes-β-Actin, beta-2 microglobulin, and RPL13a and the relative expression was calculated with respect to undifferentiated cells, D0, using the 2^{-ΔΔCt} method. Data is expressed as mean±SD, n=3.

### 2.8. Cell Viability Assay

The viability of cells was determined using the LIVE/DEAD™ cell imaging kit (Molecular Probes, Life Technologies Corp., CA) per the manufacturer's instructions. Briefly, hiPSCs cultured on 3D scaffolds were incubated with an equal volume of staining solution for 20 min at 37° C. The cells were washed in PBS and fresh medium was added to the cells. The samples (n=3) were imaged at 488 nm and 570 nm for green and red fluorescence, respectively.

### 2.9. Statistical Analysis

Statistical significance was assessed by one-way ANOVA followed by Tukey's HSD post-hoc test or by two-tailed Student's t-test to obtain p-values. All values were represented as mean±SD, and p<0.05 was considered statistically significant.

### 3. Results

### 3.1. Characterization of Co-Axial PCL-Gelatin Nanofiborous Scaffold

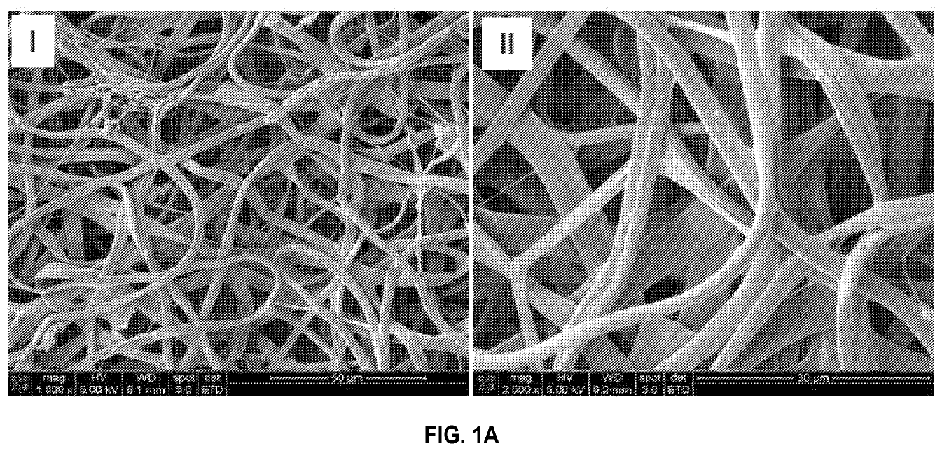

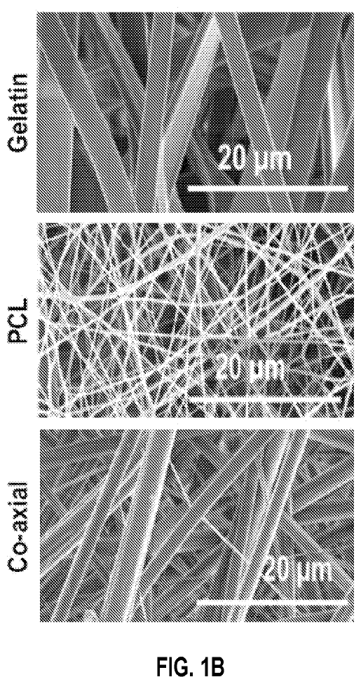

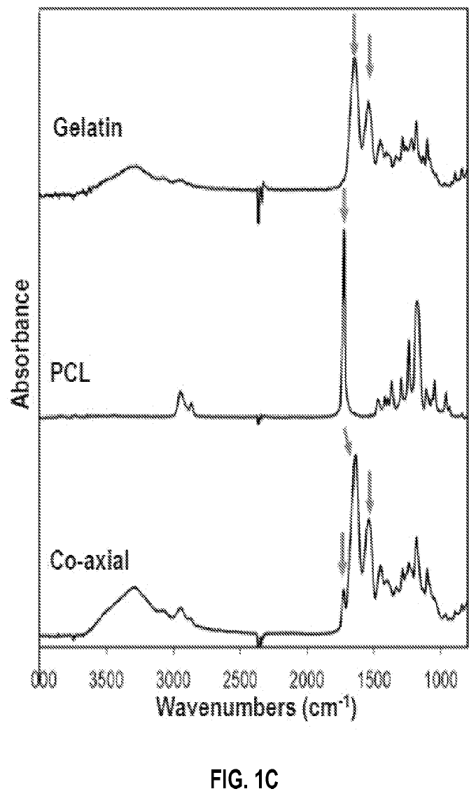

SEM images of the co-axial PCL-gelatin fibrous scaffold showed random alignment of the fibers within the patch (FIG. 1A). The co-axial fibers appeared to be more cylindrical (FIG. 1B) compared to fibers made with pure gelatin and pure PCL fibers. FTIR spectrum for the purely gelatin scaffold showed strong characteristic peaks at 1650 and 1540 cm⁻¹ (FIG. 1C, blue arrows) corresponding to the amide bonds (I and II, respectively) of gelatin. Purely PCL scaffolds displayed a characteristic peak at 1724 cm⁻¹ corresponding to its carbonyl group (FIG. 1C, red arrows). The FTIR spectrum of the co-axial PCL-gelatin scaffold was dominated by the amide I and II peaks, with a smaller peak at 1724 cm⁻¹ (FIG. 1C, blue and red arrows, respectively). These results showed the presence of both PCL and gelatin in the co-axial scaffolds.

3.2. Morphological Assessment of hiPSCs Cultured in 2D and 3D Microenvironment

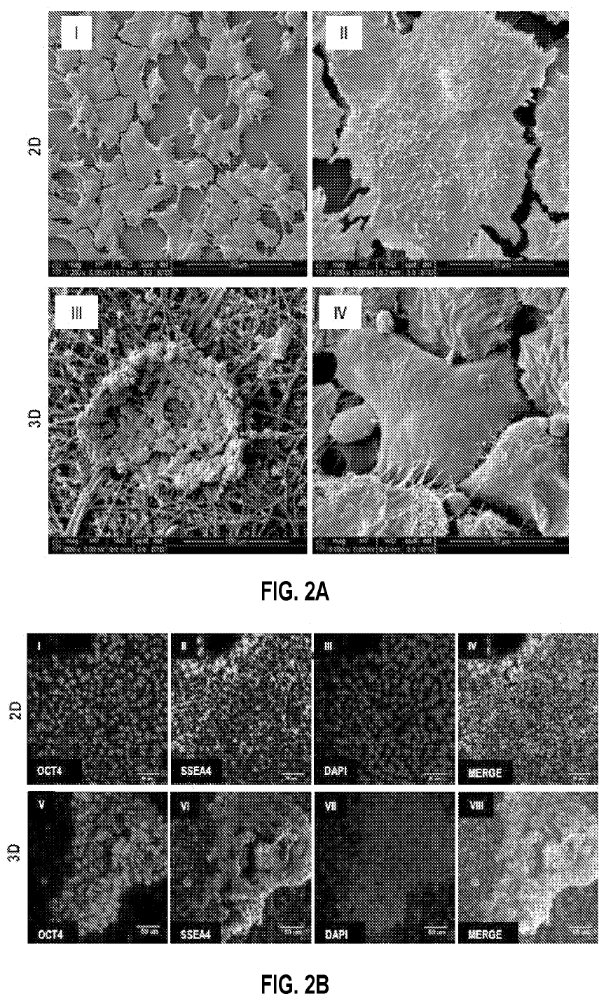

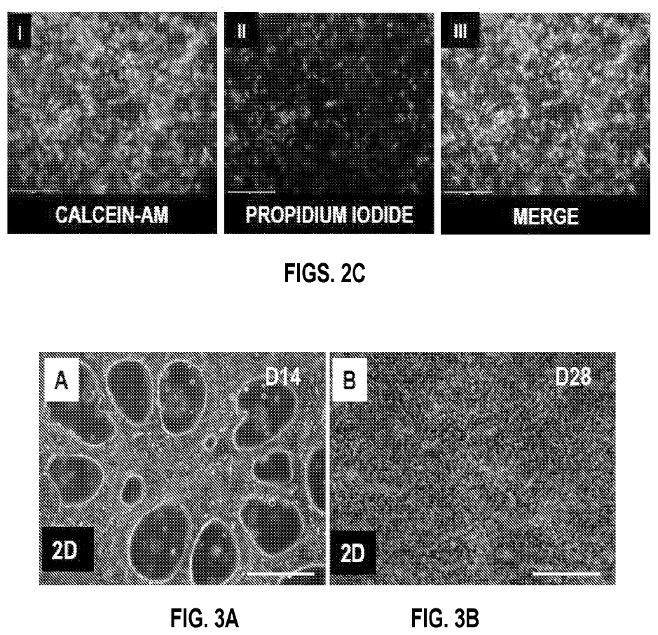

Human iPSCs cultured on 2D tissue culture plates showed 60-70% confluence in 48-72 hours. While the hiPSCs cultured in 2D showed a flattened morphology and formation of monolayer colonies (FIG. 2A, I-II), the human iPSCs cultured on 3D PCL-gelatin scaffolds showed the formation of individual dispersed colonies (FIG. 2A, III-IV). No significant difference was observed in the expression of the pluripotency markers, OCT4 and SSEA4, in hiPSCs cultured in 2D versus 3D cultures (FIG. 2B), as assessed by immunofluorescent staining.

3.3. Viability of hiPSCs Cultured on 3D Scaffolds

Cell viability was assessed following the culture of hiPSCs on 3D scaffolds for 72 hours. While a majority of the cells stained positive for Calcein-AM (green) indicating the presence of live cells, a positive staining for propidium iodide (red) was also detected in the scaffolds, indicative of the presence of dead cells (FIG. 2C). The results clearly indicated that while some cell death was observed in 3D cultures of hiPSCs, most of the cells remained viable in the scaffold. Therefore, the co-axial nature of the scaffold is non-toxic to the cells.

### 3.4. Morphological Assessment of Cardiac Differentiation in 2D and 3D Cultures

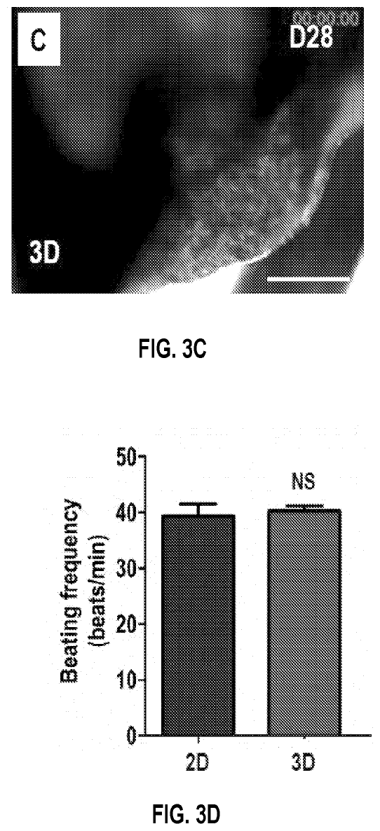

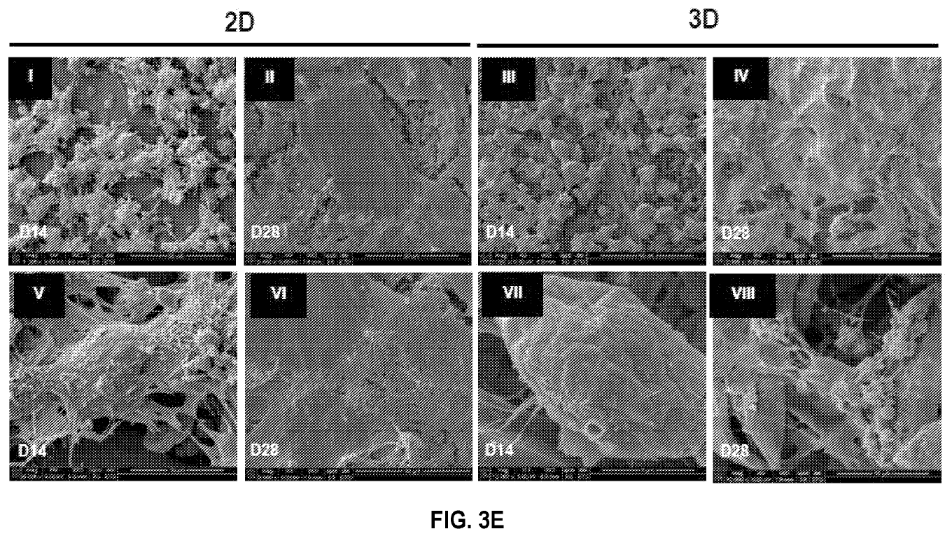

Following induction of cardiac differentiation of hiPSCs in 2D cultures, small clusters of functional contracting cardiomyocytes were observed by D8. By D14, contracting ‘sheets’ of cardiomyocytes were observed in 100% of the wells (FIG. 3A). By D28, the cardiomyocytes, differentiated from hiPSCs, formed a continuous syncytium and showed synchronous contractions (FIG. 3B). In the case of hiPSCs differentiated on 3D PCL-gelatin scaffolds, spontaneous macroscopic contractions were observed from D21 and by D28, 89% of the scaffolds showed spontaneous contractions (FIG. 3C). No significant differences in the beating frequencies were observed in hiPSCs differentiated on 2D versus 3D cultures (FIG. 3D).

Changes in cell morphology during cardiac differentiation in 2D versus 3D cultures were assessed by SEM analysis. On D14, SEM images of the 2D and 3D cultures did not show any significant differences in their morphology (FIG. 3E, I, III, V and VII). However, in the case of 3D cultures, the cells appeared to have penetrated the scaffold and wrapped themselves around the individual fibers (FIG. 3E, VII-VIII). On Day 28, the differentiated cardiomyocytes formed a monolayer with good cell-cell contact in both 2D and 3D cultures (FIG. 3E, II, IV, VI and VIII). Increased levels of ECM secretions were observed by D28 as compared to D14 in both the 2D and 3D cultures. Taken together, these results showed differentiation of hiPSCs to functional cardiomyocytes on the scaffolds.

### 3.5. Immunofluorescent Studies of Cardiac Differentiation in 2D and 3D Cultures

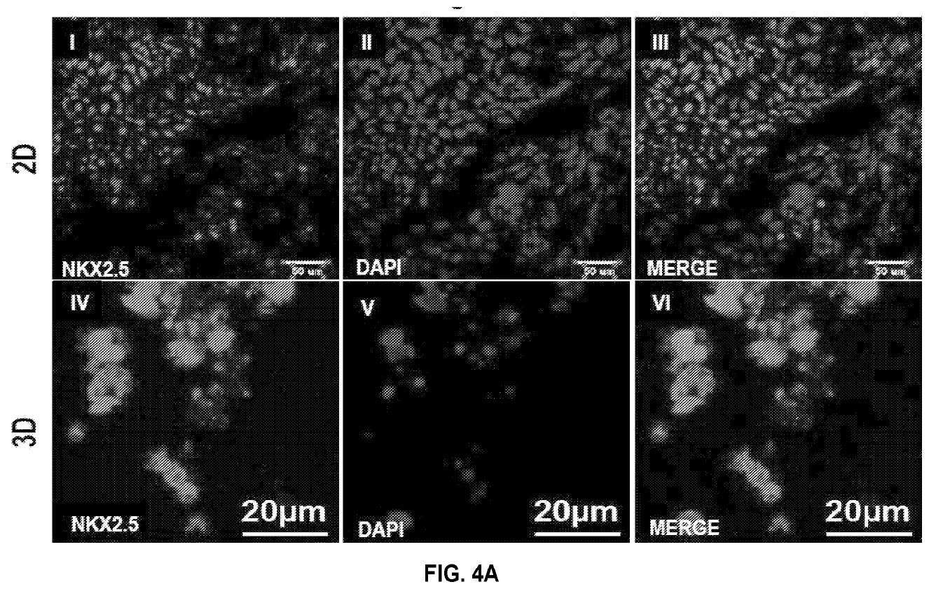

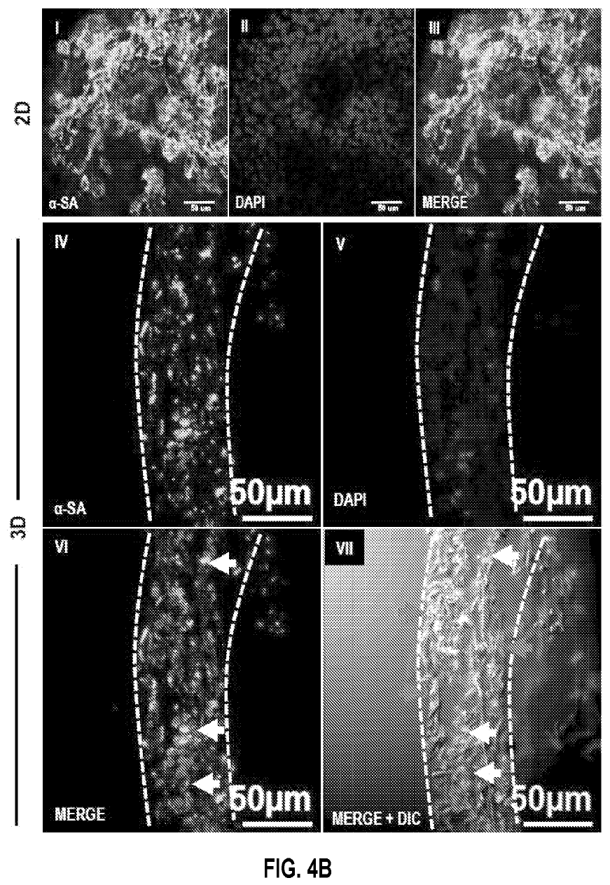

Following cardiac differentiation of hiPSCs, expression of NKX-2.5 was observed in the differentiated cells in both 2D and 3D cultures by D7 (FIG. 4A). On Day 28, the expression of cardiomyocyte marker sarcomeric alpha-actinin was observed in a majority of the differentiated cells in both 2D as well as 3D cultures (FIG. 4B). Additionally, cross-sections of 3D scaffolds on which the hiPSCs were differentiated to functional cardiomyocytes showed a uniform distribution and migration of the differentiated cardiomyocytes throughout the depth of the scaffold (FIG. 4B, IV-VII). These results demonstrate that cardiomyocytes differentiated from hiPSCs in situ on 3D scaffolds expressed markers associated with differentiated cardiomyocytes and also were distributed uniformly in the patch.

### 3.6. Assessment of Gene Expression During Cardiac Differentiation in 2D and 3D Cultures

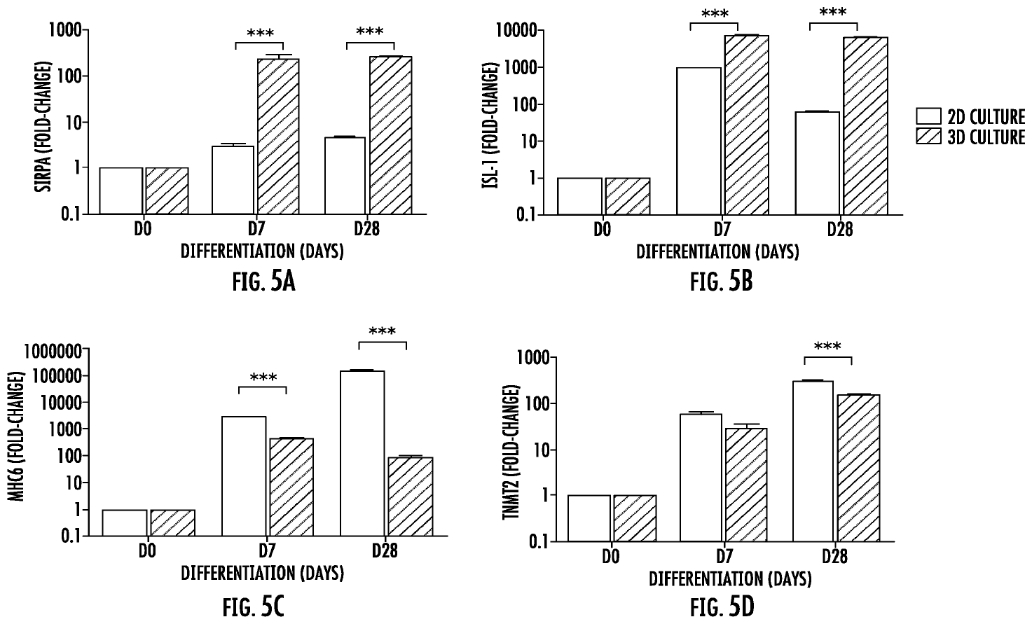

Gene expression analysis by qRT-PCR showed a significant up-regulation in the expression of Cardiac Progenitor (CP)-associated genes, SIRPA and ISL-1, and cardiomyocyte-associated genes, MHC6 and TNNT2, on D7 and D28, in both 2D and 3D cultures (FIG. 5). However, compared to 2D cultures, the CP-associated genes, SIRPA (FIG. 5A) and ISL-1 (FIG. 5B) were significantly up-regulated in the 3D cultures on both D7 and D28, as compared to their 2D counterparts. On the other hand, the expression of the cardiomyocyte-associated genes, MHC6 (FIG. 5C) and TNNT2 (FIG. 5D) was significantly up-regulated in the 2D cultures when compared to the 3D cultures on D28. These data demonstrate an enhanced differentiation of hiPSCs to cardiac progenitors in 3D cultures, when compared to their 2D counterparts. On the other hand, the data clearly showed significantly higher expression of differentiated cardiomyocyte gene expression in 2D cultures compared to 3D cultures.

### 4. Discussion

This example demonstrated the effect of co-axial PCL-gelatin nanofibrous scaffolds on the growth and differentiation of hiPSCs to functional cardiomyocytes in vitro. This study is the first one to demonstrate differentiation of hiPSCs to functional cardiomyocytes in 3D cultures using co-axial nanofibrous scaffolds. While no significant differences were observed in culturing undifferentiated hiPSCs in 2D and 3D cultures, a delay in appearance of functional cardiomyocytes was observed in the latter. On the other hand, while 3D cultures showed increased expression of cardiac progenitor-associated genes, 2D cultures showed an increased expression of cardiomyocyte-associated genes. Furthermore, although migration into the scaffold and even distribution of the differentiated hiPSC-CMs was observed in 3D cultures, a higher number of dead cells were as observed in these cultures.

This study developed a co-axial scaffold comprised of fibers with a PCL core and gelatin shell. Electrospun PCL nanofibrous scaffolds have previously been used extensively for cardiac applications. Additionally, fibrous scaffolds made from PCL have been used for differentiation of hiPSCs to neural cells as well as pancreatic p-cells. However, the reports have suggested a negative effect of PCL fibers on cell viability and function. On the other hand, when PCL was combined with natural polymers like gelatin or collagen, the scaffolds have improved biocompatibility. In fact, a core-shell structure is superior compared to a blend in terms of biocompatibility, without significant changes to the mechanical properties of the scaffold. Contradictory to this, in the current study, some cell death was observed during culture of hiPSCs on the co-axial scaffolds when compared to the 2D counterparts. In the case of 2D cultures, dead cells were regularly washed off when the cell culture medium was replaced every day. As opposed to this, in 3D cultures, the dead cells are entrapped within the scaffold and are not washed off easily during media replacement. Another plausible reason for increased cell death is the insufficiency of diffusion of nutrients and gases towards the core of the scaffolds maintained in static in vitro cultures. This problem can be addressed using dynamic culture systems.

In terms of morphology, no significant differences were observed in the morphology of the hiPSCs differentiated in 2D or 3D cultures. Differentiation of hiPSCs to spontaneously contracting cardiomyocytes in 2D cultures has previously been well-established. In the current study, spontaneously contracting ‘cardiac patches’ were also observed in culture following differentiation of hiPSCs cultured on 3D scaffolds. Visible contractions at a significantly earlier time (D8) were observed, when iPSCs were differentiated to functional cardiomyocytes in 2D cultures as compared to 3D cultures. However, no significant differences were observed in the beating frequency of cardiomyocytes four weeks (D28) after induction of differentiation between the two culture systems.

Remarkably, a delay was observed in the appearance of macroscopic contractions in human iPSCs differentiated on 3D scaffolds (D21) when compared to 2D cultures (D10). Contrary to these observations, a recent study reported appearance of macroscopic contractions by D14, following differentiation of hiPSCs to cardiomyocytes on poly vinyl alcohol (PVA) scaffolds fabricated by gas foaming. The delayed detection of contractions on the 3D scaffolds shown herein can be due to a few reasons including the ability to visualize contractions. The cells cultured on the scaffold in this study cannot be visualized under phase-contrast light microscopy, thus the precise time of the initiation of beating of the cardiomyocytes cannot be determined. In contrast, small clusters of cardiomyocytes showing weak contractions can easily be identified in 2D cultures as early as D8. It is also plausible that the force of contraction generated by the cardiomyocytes at D14 was not strong enough to translate into macroscopic contractions on the 3D scaffolds. It has been well-established that the force of contraction generated by cardiomyocytes increases with their maturation and duration in culture. Hence, the delay in appearance of contractions on the current 3D scaffold is indicative of an immature state of the cardiomyocytes differentiated from hiPSCs on 3D scaffolds or relative stiffness of the co-axial scaffold compared to normal extracellular matrix.

When compared to corresponding 2D cultures, the 3D cultures had a significantly higher expression of CP-associated genes. While the cardiomyocyte-associated genes were expressed (on both D7 and D28) at significantly lower levels indicating efficient differentiation of hiPSCs to CPs and immature cardiomyocytes on 3D scaffolds, but not to mature cardiomyocytes. However, this observation is contrary to previous reports, which showed an up-regulation of cardiomyocyte genes following cardiac differentiation of mouse iPSCs or human embryonic stem cell-derived CPs, cultured on PCL/fibrin-based scaffolds. Importantly, other studies have also shown improved differentiation, maturation and contractile gene expression following culture of hiPSC-derived cardiomyocytes in 3D cultures, as compared to 2D cultures. These studies have implicated a number of factors in the enhanced maturation of the cardiomyocytes in 3D cultures, such as (a) matrix composition, (b) fiber alignment, (c) matrix stiffness, (d) density of cells seeded onto the scaffold, (e) cells used for co-culture, and (f) electrical or mechanical stimulation. However, few studies have shown enhanced cardiac differentiation in 3D cultures as compared to 2D cultures, on aligned nanofibrous scaffolds compared to random scaffolds, and in stiffer matrices. Furthermore, a comprehensive understanding of how these factors affect the differentiation of human iPSCs to functional cardiomyocytes is currently lacking.

Another observation found in the current study was the migration and distribution of cells throughout the depth of the scaffold following cardiomyocyte differentiation on the 3D scaffolds. The hiPSC-CMs were able to migrate without the use of inducing factors; this has not been reported previously. Of importance is the fact that a gradual migration of the cells in the scaffold was observed during differentiation. It has been reported that during cardiac differentiation, hiPSCs undergo epithelial-to-mesenchymal transition followed by mesenchymal to epithelial transition in a stage-specific manner. Previous studies, using both in vitro as well as in vivo models, have shown higher migration potential of mesenchymal cells as compared to the epithelial cells. Hence, it is possible that in situ differentiation of hiPSCs to functional cardiomyocytes enabled the cells to migrate into the scaffold during the process of epithelial-to-mesenchymal transition (EMT), which occurs during the process of differentiation.

Overall, this example demonstrated that co-axial PCL-gelatin nanofibrous scaffolds can be used as a 3D platform for the culture and differentiation of hiPSCs to functional cardiomyocytes. Furthermore, efficient migration and uniform distribution of differentiated cells was observed on 3D scaffolds.

### Example 2. Nanofiber Patch for Cardiac Repair

In the United States and Canada, more than 1,000 Fontan operations are performed annually. The Fontan operation for children born with a single ventricle palliates effectively in childhood, but is associated with high morbidity and mortality in adulthood. The Fontan circuit allows blood to be oxygenated at nearly normal levels, however, the tradeoff is systemic venous hypertension and decreased cardiac output, which lead to progressive functional decline. Complications in the single ventricle population are ubiquitous, with the vast majority of Fontan patients manifesting an associated complication by their early-20's. Higher systemic venous pressure results in a cascade of physiologic derangements manifesting as lower extremity swelling, ascites, and protein losing enteropathy and plastic bronchitis in the absence of traditional systemic ventricular failure—globally described as “Fontan failure (FF).” At present, FF is treated with palliative measures, and is an indication for transplantation, as traditional heart failure therapies have not proven to be useful in this physiology. The combination of limited organ availability and the growing population of Fontan patients pose a challenge. As such, there is an increasing demand for mechanical circulatory support (MCS) as codified in the most recent heart allocation prioritization system in the U.S. Currently, one in five children is now bridged to transplant with a ventricular assist device (VAD). The use of VAD has led to significant improvements in end-organ perfusion and has decreased half of the ‘waitlist’ mortality. Despite the allocation requirements and the increased use in pediatric patients, use of MCS in the Fontan population has demonstrated limited success. Use of MCS in Fontan patients necessitates inpatient hospitalization, typically in intensive care, unlike non-congenital cohorts. Improvement in MCS outcomes and potential for successful decannulation from MCS and/or transplantation is needed. Therefore, 3-D cardiac patches seeded with stem cells can substantially enhance the contraction of the single ventricle, improving cardiac output and abrogating many of the physiologic disorders associated with the Fontan heart.

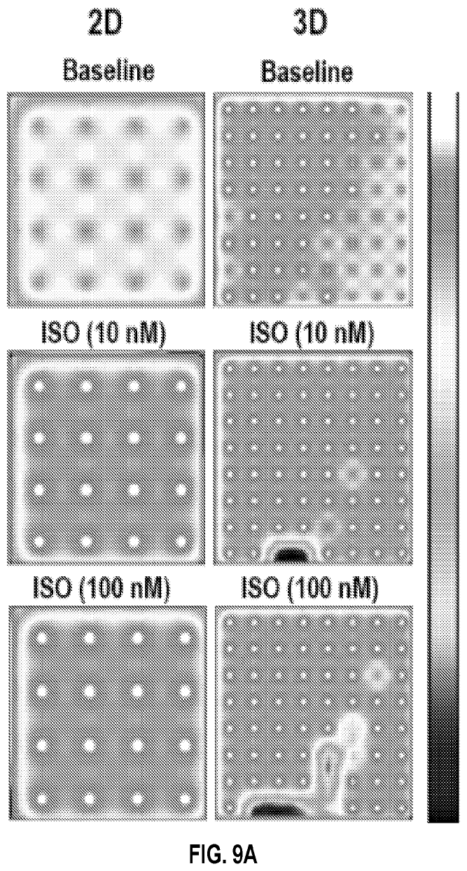

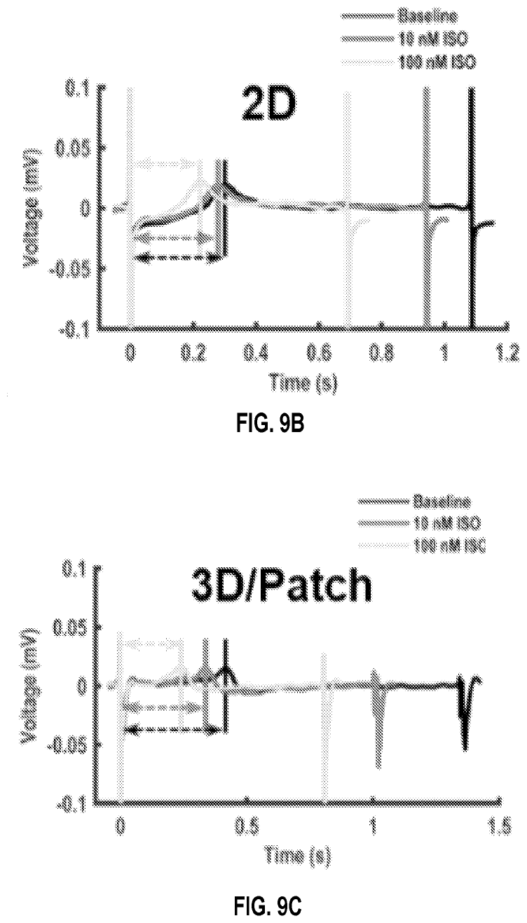

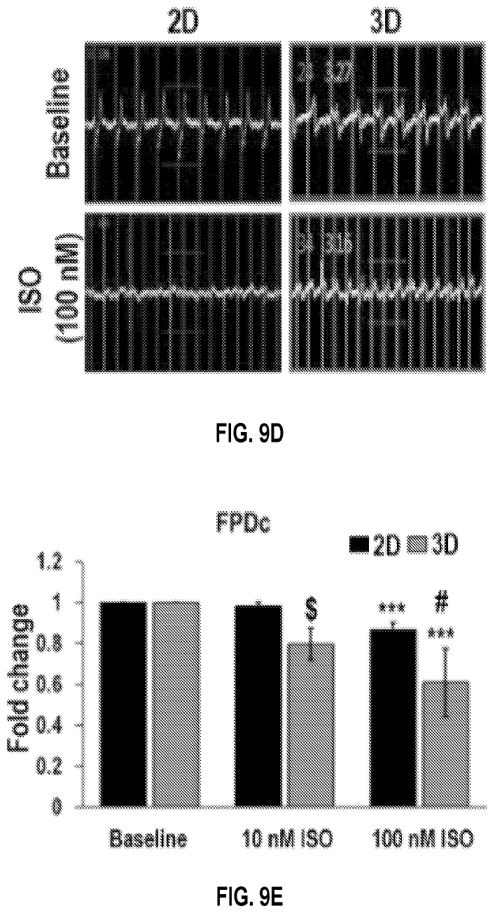

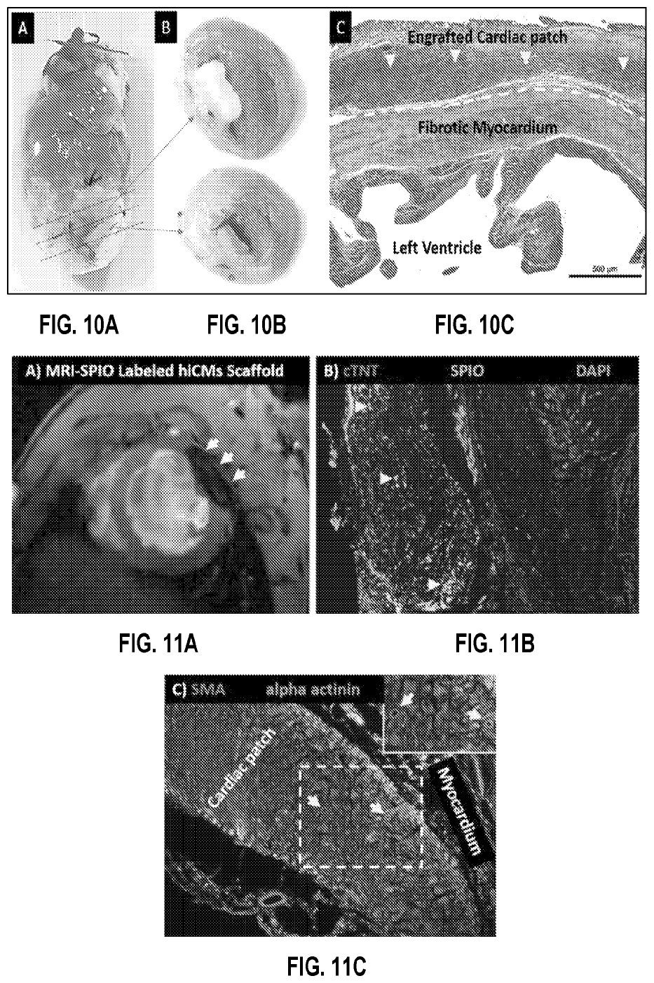

The discovery of human induced pluripotent stem cells (hiPSCs) in 2007 by Dr. Yamanaka, and the subsequent differentiation of hiPSCs to cardiomyocytes is emerging as autologous platform for cardiac repair applications. Furthermore, recent studies using hiPS-derived CPCs (hiCPCs) have shown their ability to differentiate into tri-lineage (cardiac, endothelial and smooth muscle) cells and transplantation of hiCPCs in mouse MI model attenuated fibrosis, promoted myoangiogenesis, and improved cardiac function. However, a major impediment to stem-cell therapy is poor retention and survival of transplanted cells after intramyocardial injection or intracoronary infusion leading to modest improvements in cardiac function and the effect is attributed mainly to paracrine mechanisms. Therefore, the use of scaffold/extracellular matrix is currently being investigated to improve the survival of stem cells and to provide trophic support to the ischemic heart. Electrospun nanofibers can be made from multiple polymers and the process tuned to adjust for biosafety, stiffness, tensile strength and porosity. Human-iPSC-derived cardiomyocytes (hiCMs) seeded on an electrospun aligned PLGA nanofiber scaffolds provided an anisotropic environment and increased maturation of cardiomyocytes compared with cells cultured on flat surface. Here, more robust biomimetic co-axial nanofiber scaffolds are used, which have a polycaprolactone (PCL) core and a gelatin (Gel) shell for mechanical strength and cell adhesion properties. The data strongly demonstrated that PCL-Gel co-axial nanofibrous scaffolds seeded with hiCMs showed macroscopic contractions within one-week and responded to increasing concentrations of isoproterenol (FIG. 9). Furthermore, successful engraftment and survival of hiCMs was observed upon transplantation of the cardiac scaffold onto the ischemic heart in an acute myocardial infarction (MI) model in rats (FIGS. 10 and 11). This study tests the synergistic effect of co-culturing hiCMs and hiCPCs on a cardiac scaffold and testing its efficacy in both in vitro and in vivo MI model.

Functional and Paracrine Characterization of Cardiac Scaffolds Seeded with a Combination of hiCMs and hiCPCs In Vitro.

In vitro assays are performed on cardiac scaffolds (hiCMs+hiCPCs) via a multi electrode array (MEA) to assess its contractility, field potential, and conduction velocity. Differentiation of hiCPCs into cardiomyocytes is assessed in co-culture system and expression of paracrine factors and their secretion is evaluated in the cell supernatant. The cardiac patch seeded with a combination of hiCMs and hiCPCs have increased electrophysiological function and enhanced expression/secretion of paracrine factors as compared to single cell cardiac scaffolds.

Long-Term Functional Assessment of Cardiac Scaffold Transplantation in a Rat MI Model In Vivo.

Myocardial infarction is performed in athymic nude rats and cardiac scaffold/patch (hiCMs, hiCPCs and co-cultured hiCMs+hiCPCs) is transplanted onto the ischemic heart. Cardiac function is assessed by echocardiography and MRI at 1 and 4 and 8 weeks post-MI. Transplantation of the cardiac patch onto the infarcted ventricle can improve left ventricular (LV) function and attenuate fibrosis. Furthermore, paracrine array (cytokines) and RNA-sequencing are performed on LV cardiac sections to assess changes induced in the rat heart by the scaffolds.

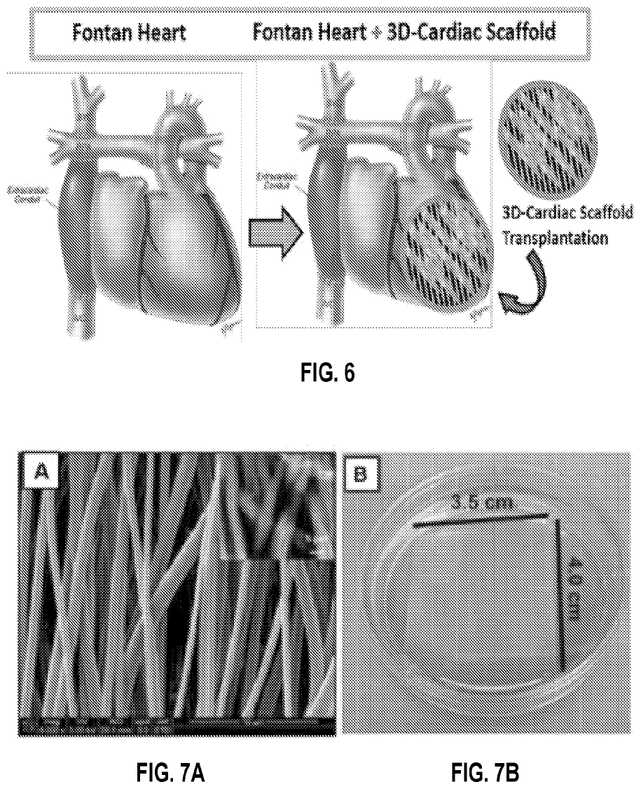

The biomimetic 3-D cardiac scaffold seeded with stem cells is applied to improve the left ventricular functional outcome in patients with Fontan failure (FIG. 6).

Relevance to Single Ventricle Heart Defects

Systolic dysfunction in “Fontan failure” is the most common late manifestation of Fontan failure recognized in the pediatric population. A systemic ventricle of “non-left ventricle” morphology in children, but not adults, carries a higher risk of unscheduled hospitalization in children compared with adults. Due to improved medical care, there is an increasing population of people with a single-ventricle. Unfortunately, a portion of these patients will develop heart failure (HF) and mechanical circulatory support (MCS) remains the only viable treatment option. Although, patients with a single-ventricle currently represent a small proportion of the total number of patients who receive mechanical circulatory support (MCS), these numbers tend to increase as the population of patients with a single-ventricle increases.

Limited case studies in children have addressed Fontan failure with the transplantation of stem cells and clinical trials are underway. One case, an 11-month-old boy with severe heart failure after Fontan with 22% EF of his systemic right ventricle, was treated with autologous bone marrow cells via an intracoronary infusion, which after one year increased his EF to 44%. Clinical trials using autologous umbilical cord blood cells are underway, which infuse and inject the cells to pediatric patients. Other studies involving stem cell transplantation in children are using bone marrow cells or peripheral stem cells. Results thus far show an increase in LVEF of about 20% after treatment. The results of these studies illustrate the increased regenerative capacity of the pediatric heart. However, results also show that intramyocardial or intracoronary infusion of cells to the heart can be inefficient, as cells are lost to the circulation or have poor engraftment, which is difficult to assess in patients.

Stem cells and tissue engineering approaches for cardiac repair. Stem cell therapy for myocardial regeneration post-MI has been investigated to a great extent in the last few decades. Adult stem cells like mesenchymal stem cells, c-kit⁺ CPCs, cardiosphere-derived cells and umbilical cord blood-derived mononuclear cells as well as PSC-derived CPCs and cardiomyocytes, have been studied for their potential use in cardiac regenerative medicine. Furthermore, clinical studies have established the safety in using these stem cell types in post-MI patients. In addition to MI, use of stem cells as a palliative therapy for a number of other cardiac conditions, including hypoplastic left heart syndrome (HLHS) has been explored with a few studies showing an improvement in cardiac function following stem cell transplantation. However, a major drawback in most of these studies has been the poor retention and engraftment of cells delivered into the host myocardium by direct intramyocardial injection. As a result, only a modest improvement in the cardiac function has been reported. To overcome this limitation, in the last decade, tissue engineering approaches making use of bioengineered scaffolds and stem cells have been developed to improve cell engraftment. A study demonstrated the use of fibrin-based scaffolds for transplantation of hiCMs in pre-clinical animal models. These studies have shown improved engraftment of the transplanted cells into the host myocardium and reduced instances of arrhythmias. Furthermore, the scaffolds also provide mechanical support to the cardiac tissue, thus preserving its structure and improving its functional output. On the other hand, Xuan et al demonstrated that intramyocardial delivery of hiCPCs attenuated fibrosis, promoted myoangiogenesis, and improved cardiac function in infarcted mice. Therefore, electrospun co-axial aligned nanofiber cardiac patches are used herein, which provides both mechanical strength (PCL) and cell adhesion (Gel) for cardiac repair. Additionally, utilizing a cardiac patch seeded with a combination of hiCMs and hiCPCs provides functional, mechanical, and paracrine support to the failing left ventricular myocardium. CPCs, known to secrete myriad growth factors and activating signaling molecules, provide paracrine support, while cardiomyocytes provides contractile support to failing heart tissue. Using hiPSC-derived cells can allow for autologous cell transplantation and an electrospun co-axial nanofiber scaffold can also lend structural support to the ventricle. Thus, transplantation of an electrospun co-axial nanofiber cardiac patch seeded with both hiCPCs and hiCMs is used herein to provide functional, mechanical, and paracrine support to a failing ventricle.

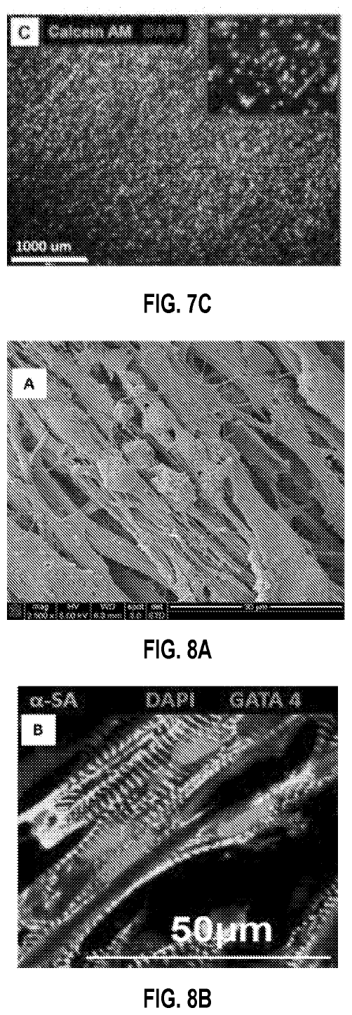

Despite the palliative interventions, the single-ventricle is poorly suited to provide long-term systemic perfusion and is prone to eventual failure. In the absence of satisfying curative treatments, stem cell therapy represents an innovative approach to the management of ventricular dysfunction in hypoplastic left heart syndrome (HLHS) patients. A recent case report demonstrated that intracoronary infusion of bone-marrow-derived mononuclear cells in a patient 23 years after the Fontan operation improved ventricular function, with maximal changes observed 3 months after the cell transplantation. The study herein brings several main innovative aspects 1) Using biomimetic aligned co-axial PCL-Gel nanofiber scaffold for seeding hiCMs (FIG. 7); 2) the data demonstrates engineered cardiac patch responding to isoproterenol (FIG. 9); 3) Survival and engraftment of transplanted patch at 4 weeks (FIGS. 10 and 11); 4) In vivo tracking of transplanted patch by MRI (FIG. 11); and 5) combination of hiCMs and hiCPCs for cardiac repair and this combination has not been tested before.

3-D Biodegradable Scaffold Seeded with a Combination of hiCMs and hiCPCs Provides Synergistic Long-Term Effect in Improving Ventricular Function and Superior Paracrine Effect in the Failing Ventricle.

Single-ventricle systolic function is improved using a biologic cardiac scaffold composed of hiCMs and hiCPCs. Assessment is performed for examining whether a co-culture seeded nanofiber scaffold has increased functionality, paracrine secretion, and survival after transplantation to the rat MI model, as compared to single cell type seeded scaffolds. Functional data determines the effect on cardiac output in terms of LVEF and fractional shortening (Echo and MRI). The addition of CPCs to the scaffold enhances paracrine signaling to support not only the transplanted cells but also the failing heart tissue.

The approach involves a combination of stem cells and biomimetic scaffolds to engineer a cardiac patch capable of delivering long-term mechanical support and paracrine signaling, when transplanted onto the infarcted LV myocardium in an acute MI model in rats. RNA-sequencing is performed in the LV cardiac sections to assess pathways associated with proliferation, cardiac muscle contraction, cardiac muscle regeneration, calcium transport and conduction systems that are impacted after an MI. Proteomic analysis of cytokines in rat heart lysates demonstrates the paracrine effects of the patch transplantation onto the ischemic heart.

1. Functional and Paracrine Characterization of Cardiac Scaffolds Seeded with a Combination of hiCMs and hiCPCs In Vitro.

In vitro assays of 3-D cardiac scaffolds are performed on MEA to assess its contractility, field potential, and conduction. Cell viability within the patch is analyzed. Expression of paracrine factors and their secretion in the cell supernatant are examined. The cardiac patch seeded with a combination of hiCMs and hiCPCs have increased electrophysiological function and enhanced expression/secretion of paracrine factors as compared to single cell type patches.

The approach involves testing coaxial nanofiber scaffolds seeded with hiCMs/hiCPCs in vitro. The co-cultured hiCMs and hiCPCs cardiac patches are evaluated. Functional assessment of the cardiac patches is performed by MEA system to record the field potentials. Furthermore, mechanical testing and SEM imaging of these cardiac patches are performed. The hiCPCs and hiCMs are co-cultured for one-week on the nanofiber scaffolds in a ratio of 1:2, respectively, with a total of 500,000 cells/cm². Similarly, cardiac patches are seeded separately with only hiCMs and only hiCPCs for comparison (FIGS. 7 and 8). The hiCPCs are procured from Fujifilm. These cells have been well characterized and are known to differentiate into cardiac, endothelial and smooth muscle cell lineages.

1.1. Electrophysiological assessment of patches: Cardiac patches are assessed for spike amplitude, beat period, field potential duration, beat irregularity, and conduction velocity using an Axion Biosciences Maestro Edge MEA system (FIG. 9). A 24-well MEA plate allows for simultaneous testing over time in a gas and temperature controlled environment. Co-culture patches are compared with hiCMs only and hiCPC only patches. These cardiac patches are treated with isoproterenol (FIG. 9), a non-selective β adrenoreceptor agonist, to asses the electrophysiological changes in field potential in co-cultured patches versus hiCMs only patch.

1.2. Assessment of cell viability, proliferation and differentiation of co-cultured cells in the patch: hiCPCs and hiCMs ARE transfected with plasmids for GFP (green fluorescent protein) and RFP (green fluorescent protein). Single cell type and co-culture patches are assessed for cell viability with calcein-AM staining, XTT, and TUNEL assay. Simultaneous detection with antibodies to KDR (hiCPC marker) and cTnT (hiCM marker) are performed to identify hiCPCs/hiCMs within the scaffold, respectively (FIG. 11B), determining if cell types are equally healthy. Differentiation of hiCPCs are assessed in the scaffold to cardiomyocytes, endothelial cells and smooth muscle cells via cTnT, CD31/vWF and α-SMA immunostaining, respectively. hiCPCs are identified by co-localization of these markers with GFP. Patches are analyzed for strength, stiffness and elastic/viscoelastic recovery using a TestResources Dynamic Mechanical Testing Rig with a biobath. At 1 and 4 weeks post cell seeding onto the patch SEM imaging illustrates mesoscale cell organization, infiltration and biodegradation of the patch.

1.3. Hypoxic induction to assess cardiomyocyte survival in co-cultured scaffolds: To assess whether co-culturing of hCPCs along with hiCMs protect cardiomycoytes under hypoxia, the cardiac scaffolds are cultured under a controlled hypoxic microenvironment (1% 02) and the cell viability is assessed by XTT and TUNEL assay.

1.4. Analysis of gene expression of co-culture patches: A cardiac gene array and paracrine-signaling array are performed on the co-cultured cell patches, as well as single cell type patches for comparison of gene expression. A custom cardiac gene array is designed to analyze 26 cardiac-related genes to investigate cardiomyocyte maturity and presence of progenitor cell population. The paracrine array (PAHS-150Z, Qiagen) analyzes expression of 84 cytokines and chemokines to explore signaling in the cells. For both arrays, total RNA is isolated from patches by the established protocol using an exogenous Luciferase mRNA spike-in (L4561, Promega) as a normalization control. Furthermore, cell lysates and conditioned media are analyzed at the protein level for 32 growth factors, cytokines, and chemokines with an ELISA array (Signosis, EA-4002). This analysis gives a clear depiction of functional paracrine signaling from each patch type.

Results: Increased contractility in hiCMs+hiCPCs co-cultured patches is observed, compared to hiCM-only. Co-culturing of hiCPCs with hiCMs improves the electrophysiological function (assessed by MEA) and cardiac gene expression. Cell death of hiCMs on the co-culture patch is decreased, when compared to hiCM-only, as a result of the paracrine effects of the hiCPCs. Cytokine and chemokine gene expression changes.

2. Long-Term Functional Assessment of Cardiac Scaffold Transplantation in a Rat MI Model In Vivo.

Since there are no available models to simulate Fontan failure, a rat MI model is used to study Fontan failure. Briefly, myocardial infarction is performed in athymic nude rats and cardiac scaffold/patch (hiCMs, hiCPCs and combined hiCMs+hiCPCs) are transplanted onto the ischemic heart after an MI. Cardiac function is assessed by echocardiography and MRI and fibrosis is assessed by Masson's trichrome staining. The data shown herein indicate that transplantation of the cardiac patch onto the failing ventricle improves left ventricular function and attenuate cardiac fibrosis.

Transplantation of cardiac scaffolds in a rat MI model is used herein. Safety and feasibility of cardiac scaffold (seeded with hiCMs) transplantation in a rat MI model were already shown (FIG. 10). The results demonstrated integration and survival of the transplanted hiCMs in the cardiac patch in the MI hearts (FIGS. 10B and 10C).

Induction of myocardial infarction: Immunocompromised athymic nude rats (NIH-Foxn1^{mu}) are divided into 6 groups (n=8/group; 1) Sham, 2) MI, 3) MI+patch only; 4) MI+hiCMs scaffold, 5) MI+hiCPCs scaffold, and 6) MI+hiCMs/hiCPCs scaffold). Cardiac patch is sutured (3 individual sutures, using 6-0 suture; see FIG. 10A) onto the epicardial heart surface, 30 minutes following a permanent LAD ligation. Animals are allowed to recover and monitored for up to 8 weeks post-MI. To track engraftment and survival of transplanted cells in the cardiac patch, hiCMs are transfected with RFP and GFP and both cells are labeled with SPIO particles for in vivo tracking by MRI as shown in FIGS. 11A and 11B.

2.1. Cardiac patch-induced improvement in cardiac function: Echocardiography is performed to assess the cardiac function at baseline, 1, and 4 and 8 weeks after cardiac patch transplantation. Cardiac function is determined by Echocardiography and magnetic resonance imaging (MRI). Transplanted cells are detected in heart slices with an antibody that reacts with human nuclear factor but does not react with rat, which demonstrates cell survival. Concurrent staining with KDR and cTNT determines transplanted cellular identity as hiCPC or hiCM, respectively.

2.2. Magnetic resonance imaging for tracking transplanted cardiac patch in vivo: MRI imaging is performed at 4 and 8 weeks post-cardiac patch transplantation to identify hiCMs/hiCPCs labeled with dragon green fluorescent superparamagnetic iron oxide nanoparticles (SPIO; FIGS. 11A and 11B) using Horizontal bore magnet at 9.4 T.

2.3. Assessment of cardiac fibrosis, cell engraftment and angiogenesis: At 8 weeks after cardiac patch transplantation, rats are euthanized and hearts are excised, fixed, and processed for immunostaining. Cardiac fibrosis is assessed by Masson's-trichrome staining (FIG. 11C). To assess long-term survival of cells in the cardiac patch cells, immunostaining is performed to identify the engrafted cells in the heart with human nuclear factor antibody staining (this reacts only to human cells) and anti-RFP/GFP staining of transfected cells. Immunostaining for angiogenesis is performed by CD31/alpha-SMA/Lectin staining (FIG. 11C). Furthermore, tissue homogenate is analyzed for expression of 23 growth factors, cytokines, and chemokines with a rat-based ELISA (Signosis, EA-4006).

2.4. RNA sequencing in cardiac tissue post-cardiac patch transplantation: paired-end sequencing is performed on the total RNA obtained from the rat hearts post-MI or cardiac patch transplanted groups (Sham, MI, MI+hiCMs scaffold, MI+hiCPCs scaffold, and MI+hiCMs/hiCPCs) at 8 weeks post-MI. The reads are trimmed following a quality control analysis using FATQC. The trimmed reads are then disambiguate and map against the human reference genome and rat reference genome from NCBI using BBMap tool. This helps isolate reads that uniquely map to each species from the mixed species RNAseq data. The reads that uniquely map to each species' genomes are then be re-aligned to their respective genomes using the splice aware RNASeq aligner HISAT2 (v2.2.0) with default settings to generate binary alignment map (BAM) files. The BAM files are used to determine gene-level and exon-level counts using the SummarizeOverlaps function from the

GenomicAlignments package in R for each species. The counts are used to determine differentially expressed genes using DESeq2. Pathways enriched among the significantly differentially expressed genes (adjusted P-value≤0.05) are identified using Ingenuity Pathway Analysis (Qiagen). In parallel, Gene Set Enrichment Analysis (GSEA) is used and performed using SeqGSEA, to include differential splicing and differential gene expression in pathways analysis in the rat hearts following the different treatments.

Cardiac scaffold transplantation can improve the cardiac function in rats subjected to acute MI. It was shown that hiCM-derived exosomes possess angiogenic effects in vitro, which aids in the repair of the post-MI heart. The co-cultured patch has a synergetic effect on paracrine signaling and protection of cardiomyocytes against hypoxia. Many studies of stem cell transplantation have shown poor cell survival, with <10% of cells remaining after 2 weeks. However, the data here (FIG. 11B) demonstrated detection of hiCMs in the patch 4 weeks after MI. Therefore, viable cell patches can be detectable at 8 weeks. This study establishes the efficacy, safety, and feasibility cardiac scaffold transplantation for translational studies in a large animal porcine model. The RNAseq data show pathways associated with proliferation, cardiac muscle contraction, cardiac muscle regeneration, calcium transport and conduction systems to be impacted in the MI rats compared to the Sham and Patch treated rats. In addition, pathways associated with fibrosis is impacted when MI rats get a surgical patch with hiCMs and/or additional hiCPCs. In parallel, expression changes in the graft when both hiCMs and hiCPCs are used together as compared to use of individual cell type patches.

### Example 3. Fabrication and Testing of Tissue-Engineered 3-D Aligned Coaxial Nanofiber Cardiac Patch

Recent advances in cardiac tissue engineering have shown that human induced pluripotent stem cell-derived cardiomyocytes (hiPSC-CMs) cultured in a 3-D micro-environment exhibited superior physiological characteristics compared with their 2-D counterparts. These 3-D cultured hiPSC-CMs have been used for both drug testing as well as cardiac repair applications. However, the fabrication of cardiac scaffolds with the desired mechanical strength without compromising its biomimetic properties, remains a challenge. In the current study, an aligned PCL-Gelatin coaxial nanofiber patch is fabricated using electrospinning. The structural and mechanical properties of the patch were assessed by SEM, ICC, FTIR-spectroscopy, and tensile strength analysis. hiPSC-CMs were cultured on the aligned coaxial patch for two weeks and their viability (LDH assay), morphology (SEM, ICC), and functionality (calcium cycling, MEA) were assessed. Furthermore, particle image velocimetry (PIV) and microelectrode array (MEA) were used to evaluate the cardiotoxicity and physiological functionality of the cells in response to cardiac drugs. Nanofibers patches were comprised of highly aligned core-shell fibers with an average diameter of 578±184 nm. Acellular coaxial patches were significantly stiffer than gelatin alone with an ultimate tensile strength of 0.780±0.098 MPa, but exhibited gelatin-like biocompatibility. Furthermore, hiPSC-CMs cultured on the coaxial patch showed an elongated, rod-shaped morphology and well-organized sarcomeres, as observed by cardiac Troponin-T and α-Sarcomeric actinin expression. Additionally, hiPSC-CMs cultured on these patches formed a functional syncytium evidenced by the expression of Cx-43 and synchronous calcium transients. Moreover, MEA analysis showed that the hiPSC-CMs cultured on aligned patches showed an improved response to cardiac drugs like isoproterenol (ISO), verapamil (Vera), and E4031, compared to the corresponding 2D cultures. Overall, these results demonstrated that an aligned, coaxial 3-D cardiac patch can be used for culturing of hiPSC-CMs. These biomimetic cardiac patches can further be used as a 3-D in vitro cardiac tissue platform to perform drug testing, cardiotoxicity studies, a model for “clinical trials in a dish” and for in vivo cardiac repair applications for treating myocardial infraction.

1. Introduction

Cardiovascular diseases (CVDs) are the number one cause of morbidity in North America. However, the development of therapeutics for CVDs has been limited by the unavailability of efficient model systems for drug screening and toxicology studies. Most pharmacological studies make use of primary cell lines or model organisms like rodents, rabbits, pigs and non-human primates for assessing the effect of putative drug molecules. Small animal models like rodents, which have been extensively used for pre-clinical cardiovascular drug testing, are not ideal models, since their cardiomyocytes differ significantly from humans in their structure and function. On the other hand, large animal models (pigs and non-human primates) are a good system since their cardiovascular system and the associated hemodynamics is similar to humans. However, the costs associated with their housing, maintenance, and ethical concerns make them less favorable for pre-clinical drug testing applications. While primary cultures of adult cardiomyocytes are a good, cost-effective system to study drug effects in vitro, their use is restrained by their limited availability and lack of efficient culture protocols.

In this context, human induced pluripotent stem cell-derived cardiomyocytes (hiPSC-CMs) have become increasingly popular for use as an in vitro model system. These cells have shown great potential in developing strategies for cardiac repair. Additionally, the advances in techniques for hiPSC generation, ease of scalability of hiPSC-CMs, and development of next-generation genetic manipulation techniques make hiPSC-CMs an attractive model for the development of patient-specific personalized precision medicine. Hence, hiPSC-CMs have become increasingly popular as an in vitro model for cardioprotective and cardiotoxic drug screening). However, in most of these studies, hiPSC-CMs used were cultured in two-dimensional (2-D) culture dishes. This 2-D culture system has been shown to have a few drawbacks: (a) immature phenotype of hiPSC-CMs in 2-D cultures, (b) heterogeneity of the cells in culture, (c) lack of alignment of hiPSC-CMs, and (c) differential response to drug treatment. However, recent studies have shown improved function and maturation of hiPSC-CMs in 2-D cultures with increased culture time or electrical and mechanical stimulation.

In view of the above, 3-D culture systems have been shown to improve the maturation as well as functionality of hiPSC-CMs. Cells cultured in a 3-D environment are shown to have better physiological characteristics and more closely resemble native tissue than the same cells grown in the classical 2-D culture flasks. Hence, 3-D cultures provide for a better and more relevant model for cardiotoxicity and drug screening studies. The 3-D models currently explored in the case of the CVDs are as follows. a) hydrogel-based engineered heart tissue b) self-assembling spheroids formed via hanging drop method c) cardiac cell-sheets and d) bioengineered scaffolds. Of these, the scaffold-based model has been extensively studied for the development of engineered heart tissues.

3-D scaffolds have been fabricated using different bioengineering techniques, like microfluidics, 3-D bioprinting, gas foaming and electrospinning. Of these, electrospinning provides for a superior fabrication technique to ensure reduced batch-to-batch variation, better uniformity within scaffolds, nano-dimensional architecture similar to cardiac tissue, and controlled alignment of nanofibers. Further, nanofiber-based scaffolds have been fabricated using different natural and synthetic biocompatible materials. It has been reported that scaffolds made using natural polymers like gelatin and collagen exhibit efficient cell adhesion but poor mechanical properties, while the scaffolds made using synthetic polymers like poly lactic-co-glycolic acid (PLGA), polylactic acid (PLA) and polycaprolactone (PCL) have poor biomimetic and cell adhesion properties but improved mechanical support. In the present study, an aligned coaxial nanofibrous scaffold was fabricated, with nanofibers having a PCL core with a gelatin shell. The PCL imparts mechanical strength while gelatin provides the required biomimetic properties, thereby improving cell attachment. This aligned coaxial nanofibrous scaffold was seeded with hiPSC-CMs to obtain a functional 3-D ‘cardiac patch’, which was then used for drug screening and toxicity studies.

2. Materials and Methods

2.1 Fabrication of PCL-Gelatin Aligned Coaxial Nanofibrous Patch

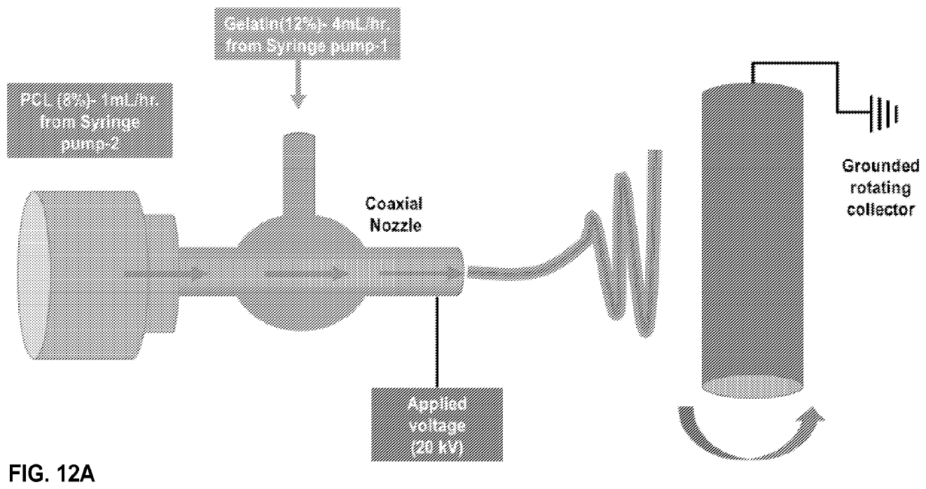

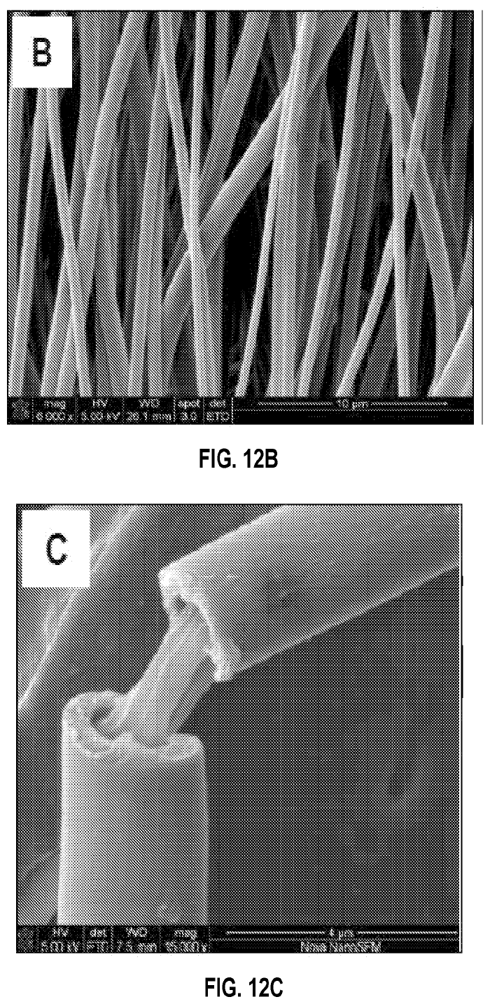

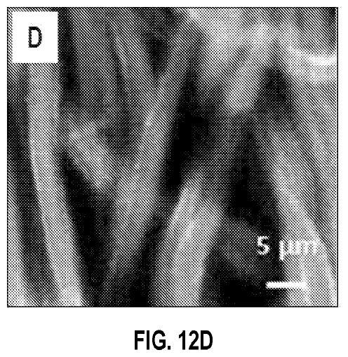

Gelatin (12% w/v) [gelatin from bovine skin, Sigma-Aldrich, St. Louis, Mo.] and PCL (8% w/v) [Sigma-Aldrich, St. Louis, Mo.; Mn=42,500] solutions were prepared in 1,1,1,3,3,3-hexafluoro-2-propanol. The gelatin and PCL solutions were fed to the outer (at a flow rate of 4 ml/h) and inner tube (at a flow rate of 1 ml/h), respectively, of the coaxial spinneret as shown in FIG. 12A. The distance between the spinneret tip and the grounded rotating collector was maintained at 20 cm and a 20 kV voltage was applied at the spinneret tip. The aligned coaxial nanofibers collected were dried inside a chemical fume hood overnight, to remove the remnant solvent.

The morphology of the nanofibrous patch was assessed by scanning electron microscopy. To confirm the coaxial morphology of the nanofibrous patch, gelatin and PCL solutions were mixed with 1% w/v fluorescein (Sigma-Aldrich, St. Louis, Mo.) and rhodamine (Sigma-Aldrich, St. Louis, Mo.), respectively, and nanofibrous patches were fabricated as mentioned above and imaged using a confocal microscope (Olympus FV3000 Confocal microscope).

Before confocal imaging, mechanical testing, and cell seeding, patches were cross-linked, sterilized, and hydrated. Briefly, patches were cut into the desired diameter using biopsy punches (8 mm) and treated with 7 mM 1-ethyl-3-(3-dimethylaminopropyl)carbodiimide hydrochloride (EDC) solution in ethanol for 24 hours, followed by incubation in 70% ethanol for hydration and sterilization. The patches were then washed in PBS, two times for 24 hours each. Patches were then used for confocal imaging, mechanical testing, and cell seeding.

2.2 Fourier-Transform Infrared Spectroscopy (FTIR) Studies

Surface chemical analysis of the gelatin-only, PCL-only, and coaxial patches was performed using attenuated total reflection Fourier transform infrared spectroscopy (ATR-FTIR) between the range of 400-4000 cm⁻¹ (Thermo Nicolet Nexus 670 FTIR spectrometer, MN). Approximately 25-40 scans were performed on the three types of patches.

2.3 Mechanical Testing

The mechanical properties of acellular aligned PCL, gelatin, and PCL-gelatin coaxial nanofibrous patches were tested using a Universal Test Machine (TestResources 100R, Shakopee, Minn.) as per American Society for Testing and Materials (ASTM) D638 Type V, using the protocol described previously. For aligned gelatin and coaxial patches, tests were performed after crosslinking in EDC solution and further hydration in PBS. For aligned PCL patches, incubation was carried out in ethanol (to mimic the crosslinking process) before hydration in PBS. The thickness of each sample was measured using digital calipers. Samples were strained (along the axis of alignment) at a grip speed of 2 mm/sec until failure. Samples were made using a dog-bone shaped punch, 3 mm gauge width, and 10 mm gauge length. The thickness of each sample was quantified using digital calipers. Stress-strain curves were generated for each sample and the tensile strength and Young's modulus were determined and reported as mean t SD. Tensile strength was determined at the point of greatest stress, before failure. Young's modulus was calculated from the stress-strain curve using linear regression analysis for the first linear region of the curve (where R²˜0.98) past the toe-in region, if present.

2.4 Culturing and Maintenance of hiPSC-CMs on Aligned Coaxial Nanofibrous Patches

The hiPSC-CMs were obtained from Fujifilm Cellular Dynamics International (CDI, WI, USA, Cat #R 1007). The cells were plated in a sterile 6-well plates according to the manufacturer's protocol and after 48 hours, cells were cultured in CDI hiPSC-CMs maintenance medium in a humidified atmosphere at 37° C. at 5% CO₂.

Once the hiPSC-CMs showed contractions, the cells were seeded onto aligned coaxial nanofibrous patches. For this, sterile cross-linked 8 mm aligned coaxial patches were transferred onto N-terface (Winfield Labs, TX, USA) and coated with 30 μl of fibronectin (50 μg/ml) for 1 hour in a humidified atmosphere at 37° C. The patches on N-terface were then transferred onto sterile sponges soaked in the hiPSC-CMs culture medium placed in a 100 mm culture dish. The hiPSC-CMs were harvested using 0.25% trypsin-EDTA and seeded onto the coaxial patches at a final density of 1×10⁶ cells/cm² (50 μl of cell suspension/patch) and incubated at 5% CO₂ at 37° C. for 1 hour. After 4-6 hours, the aligned coaxial cardiac patches (aligned coaxial nanofibrous patches seeded with hiPSC-CMs) were transferred to 6-well plates (1 patch/well) containing 2 Ml CDI hiPSC-CMs maintenance medium. The medium was replaced every alternate day for two weeks.

2.5 Scanning Electron Microscopy (SEM)

SEM was used to observe the morphology of the aligned PCL, gelatin, and PCL-gelatin coaxial nanofibrous patches. SEM was also performed on aligned coaxial cardiac patches to determine the distribution and alignment of the seeded hiPSC-CMs. For these studies, the samples were prepared. In brief, the cardiac patches were fixed in 4% paraformaldehyde (PFA, MilliporeSigma, WI, USA) for 1 hour. Patches were then washed with de-ionized water and further dehydrated by gradually increasing ethanol concentration (50%, 70%, 80%, 95%, and 100/6). The patches were then dried chemically using various gradients of hexamethyldisilazane. Finally, patches were subjected to gold-palladium coating using a sputter-coater (Pelco Model 3) and imaged on FEI NOVA nanoSEM.

2.6 Calcein-AM Staining of hiPSC-CMs

CellTrace™ Calcein Green, AM (Cat #C34852) was procured from Invitrogen (Life Technologies corporation OR, USA) and 1 mM stock was prepared in DMSO. hiPSC-CMs were incubated with 250 nM Calcein-AM for 30 min at 5% CO₂ in a humidified atmosphere at 37° C. Then hiPSC-CMs were washed with maintenance medium three times and imaging was performed on an EVOS™ FL Auto 2 Fluorescent microscope (ThermoFisher, CA, USA).

2.7 Lactate Dehydrogenase (LDH) Assay

To measure the cytotoxicity of the aligned coaxial patch on hiPSC-CMs, LDH assay was performed using the in vitro toxicology assay kit, lactic dehydrogenase based (Cat #TOX7-1KT, MilliporeSigma, WI, USA). The culture medium was collected from hiPSC-CMs cultured in tissue culture plates or on aligned coaxial patches after 48 hours of culture and LDH release assay was performed according to the manufacturer's protocols. Background and primary absorbance of the plate were measured on a spectrophotometer (2030 Multilabel Reader, Victor® x3, PerkinElmer Inc., MA, USA) at 690 nm and 490 nm, respectively. The assay was performed in quadruplicate (n=4) and data obtained was analyzed on WorkOut 2.5 (build 0428, PerkinElmer Inc., MA, USA), by subtracting background absorbance from primary absorbance.

2.8 Immunostaining

Immunofluorescence staining was performed to analyze the expression of cardiac markers in aligned coaxial cardiac patches. Briefly, aligned coaxial cardiac patches, at two weeks, were washed twice with PBS and fixed with 4% PFA for 10 min at room temperature. The patches were then washed twice with PBS and incubated in blocking buffer (PBS, 5% normal goat serum, and 0.3% Triton X) for 1 hour to block non-specific antibody binding. Following this, the patches were incubated with anti-α-sarcomeric actinin (A7811, MilliporeSigma, WI, USA), anti-GATA4 (PA1-102, ThermoFisher Sc., MA USA), anti-Troponin-T (HPA017888, MilliporeSigma, WI, USA), and anti-Connexin-43 (MAB 3067, MilliporeSigma, WI, USA) antibodies, overnight at 4° C. and after which the patches were washed thrice in PBS, 5 mins. each. Cells were then incubated with the corresponding secondary antibodies conjugated either with Texas Red or FITC against rabbit (1:5000, 8889S, Cell Signaling, MA, USA) or mouse (1:5000, 4408S, Cell Signaling, MA, USA) for 1 hr at room temperature in dark and. washed thrice in PBS. Nuclei were counterstained with NucBlue (R37605, Invitrogen, CA, USA). Finally, the patches were washed thrice with PBS, transferred onto slides and mounted with ProLong™ Glass Antifade mounting medium (Cat #P36984, Invitrogen, MA, USA). Imaging was performed on a confocal microscope (Olympus FV 1000 spectral, Olympus Corporation, PA, USA) and images were processed using the Olympus FLUOVIEW Ver. 4.2a Viewer.

2.9 Assessment of Calcium Cycling in hiPSC-CMs

Calcium transients were imaged in hiPSC-CMs cultured on fibronectin-coated glass coverslips and aligned coaxial cardiac patches using the calcium-binding dye Fluo-3, AM (F1242, Invitrogen, MA, USA). For staining, the hiPSC-CMs cultured on coverslips or patches were washed three times with Dulbecco's Modified Eagle's Medium (DMEM) and incubated in 5 μM Fluo 3-AM in DMEM for 1 hour in dark at 37° C., 5% CO₂ in a humidified atmosphere. The cells were then washed three times in DMEM and incubated for an additional 30 minutes in serum-containing medium at 37° C., 5% CO₂ in a humidified atmosphere. The cell culture plate was then placed on the microscope (Leica Microsystems, Wetzlar, Germany) stage to record a movie using Leica Application Suite X 3.0.6.17580 software.

2.10 Particle Image Velocimetry (PIV)

Cross-correlation (“PIV”) analysis of images. Cell contractility kinetics were assessed using the optical flow/PIV method. Briefly, a motion pattern (velocity field) captured on a pair of images was calculated by dividing the first image into overlapping tiles, each 64 pixels wide. The second image was then scanned pixel-by-pixel, by shifting an equally sized (64×64 pixels) window. The most similar (by Euclidean distance) tile on the second image was then assumed to be the location where the pattern in the first image moved. The resulting displacement vectors characterizing each image tile were then interpolated and denoised by a thin-plate spline fit, yielding a coarse displacement field. The coarse estimate was used to construct a second, higher resolution displacement field. In this second step, the cross-correlation search for pattern similarity was repeated with tiles that were only 32 pixels wide but in a much smaller search area allowing only for 4-pixel displacements around the location predicted by the coarse displacement field.

Beat patterns. To determine the beat patterns, a suitable reference image taken at time t*, which is a frame between two contraction cycles, where movement is minimal: V (t)>=V(I*), in a motion-free state, was first identified. This reference image was then compared to all other images of the recordings with cross-correlation (“PIV”) analysis. The result is a series of displacement vector fields d(t,x), which estimate for each time point t and location x the total movement (magnitude and directionality) relative to a resting state. For each time point t the beat pattern D(t) is the spatial average of the magnitude of d(t,x) as D(t)=<Id(t,x)|>_x, where < . . . >_x denotes spatial averaging over all possible locations x.

Frequency analysis. Fourier spectra were calculated from D(t) beat patterns using the discrete Fourier transform algorithm. Power densities were calculated as the magnitudes of the squared Fourier spectra, and indicate periodicity within the signal in the form of peaks at the corresponding frequencies. When the analyzed signal is not a pure sine wave, harmonics are expected to appear at integer multiples of the fundamental frequency f(2f, 3f, etc). The magnitude of a peak in the power spectrum indicates the amplitude of the signal oscillating with the corresponding frequency.

Convergence analysis. Cell layers often move passively without actively contracting. The optical flow-based method does not distinguish between active and passive (elastic response of the adjacent cell layer) contractility. To identify contractile centers, the convergence maps of the displacement field was estimated as its negative divergence from optical flow data d(t,x). The procedure localizes contraction centers, which often do not correspond to areas with high displacement values.

2.11 Functional Characterization Using a Multi-Electrode Array (MEA) System

The field potentials of hiPSC-CMs cultured in 2-D or on aligned coaxial patches were measured using an MEA system. For this, hiPSC-CMs were either cultured directly on 24-well MEA plates (M384-tMEA-24W, Axion Biosystems, GA, USA) having 16 PEDOT microelectrodes per well (as described previously) or on 8 mm aligned coaxial patches cultured for two weeks. The aligned coaxial cardiac patches were transferred into a sterile 6-well MEA plate (M384-tMEA-6W, Axion Biosystems, GA, USA) having 64 PEDOT microelectrodes per well. The plate was equilibrated in the MEA system (Maestro Edge, Axion Biosystems, GA, USA) for 30 minutes in 5% CO₂ with a humidified atmosphere at 37° C. For the patches, excess culture medium was removed to facilitate better contact with the electrodes. The baseline was recorded for each well for 5 minutes. After which the hiPSC-CMs in 2-D as well as the cardiac patches were treated with different cardiac drugs: (a) Isoproterenol (10 nM and 100 nM) (b) Verapamil (0.1 μM and 0.3 μM) and (c) E4031 hydrochloride (50 nM and 100 nM). The stock solutions of all drugs were prepared in dimethylsulphoxide (DMSO). The plates were equilibrated for 5 mins after the addition of drugs and the field potentials were recorded for 5 minutes for each drug treatment. AxIS Navigator™ version 1.4.1.9 was used for data recording while CiPA™ analysis tool version 2.1.10 (Axion Biosystems, GA, USA) was used for data analysis. The beat period, field potential duration (FPD), spike amplitude and the incidences of arrhythmias were calculated. Further, the Fredericia's correction was applied to the FPD, to interpret the effect of drugs on the QT interval. Data are expressed as mean±SD (n=3).

2.12. Statistical Analysis

Data acquired is expressed as mean±SD. Statistical significance was determined using one-way ANOVA. All pairwise multiple comparison procedures were performed by the Holm-Sidak method. p-value<0.05 was considered statistically significant.

3. Results

3.1 Fabrication of Aligned Nanofibrous Coaxial, PCL, and Gelatin Patches

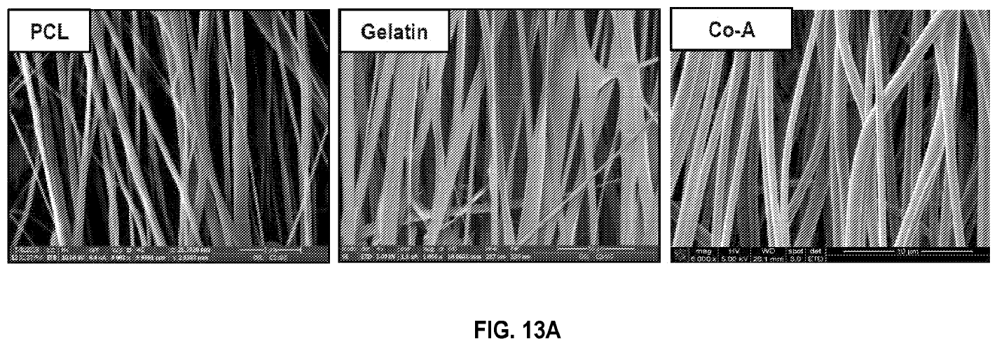

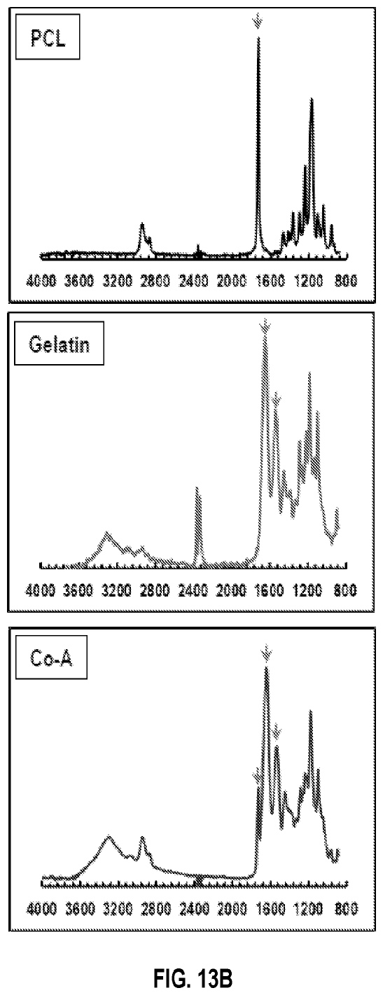

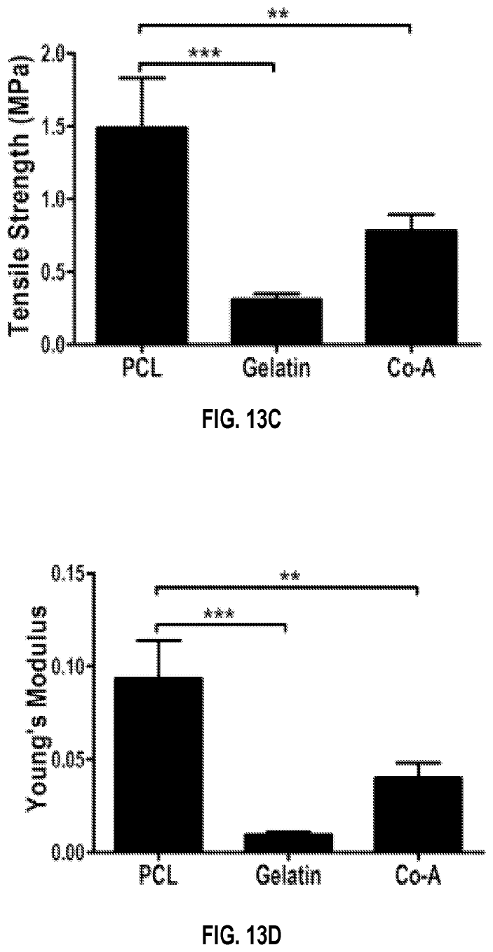

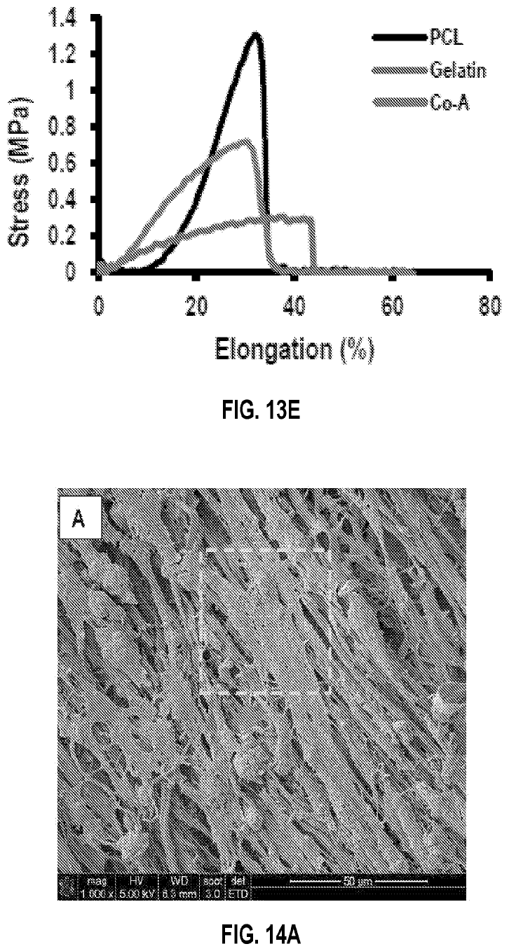

Aligned nanofibrous PCL-gelatin coaxial patches were successfully fabricated using the electrospinning technique (FIG. 12A). The nanofibers in the coaxial patches fabricated showed good parallel alignment with a mean diameter of 578±184 nm (FIG. 12B). The overall mean thickness of the aligned coaxial nanofibrous patches was 115±11 μm. SEM image of a single nanofiber showed a core-shell structure indicating successful coaxial morphology (FIG. 12C). Further, confocal microscope images of the coaxial patches after mixing of rhodamine and fluorescein with PCL and gelatin, respectively, validated the coaxial morphology. These images clearly showed the presence of PCL (red) in the core and gelatin (green) in the shell (FIG. 12D). The confocal image of the aligned coaxial patch showed that the nanofibers had a core diameter of 2.21±0.50 μm. The increased mean diameter of nanofibers in confocal images, when compared with the mean diameter observed in SEM images can be a consequence of hydration during crosslinking and washing of the patches before confocal imaging. Additionally, a comparison of the SEM images of only PCL and only gelatin nanofibrous patches with coaxial patches clearly showed that the coaxial nanofibers had more uniformity and cylindrical morphology (FIG. 13A). Also, the FTIR analysis of the PCL-only and gelatin-only patches showed peaks corresponding to C═O ester of PCL at 1722^{cm-1} (green) and peaks corresponding to the amide groups at 1544^{cm-1} and 1657^{cm-1} (brown), respectively (FIG. 13B). On the other hand, the coaxial patches showed all three peaks (FIG. 13B), further reiterating the presence of both polymers in the nanofibers.

3.2 Mechanical Testing of Aligned Coaxial Patches

The mechanical strength of PCL-only, gelatin-only, and the PCL-gelatin coaxial patches was determined by tensile testing. The stress-strain curves obtained showed that the aligned PCL-only patch had the highest values for both tensile strength (1.490±0.290 MPa) as well as Young's modulus (0.093±0.017 MPa) (FIGS. 13C and 13D). This was followed by the aligned coaxial patch, which had a tensile strength and Young's modulus of 0.780±0.098 MPa and 0.039±0.007 MPa, respectively (FIGS. 13C and 13D). The aligned gelatin-only patches had the least mechanical strength with tensile strength and Young's modulus of 0.308±0.032 MPa and 0.009±0.001 MPa (FIGS. 13C and 13D), respectively. However, in terms of percent elongation, gelatin showed the highest elongation, while no significant difference was observed between the PCL and coaxial patches (FIG. 13E). Taken together, these results showed that the PCL patches had the highest mechanical strength while gelatin had the highest elongation. The aligned coaxial patches had better mechanical properties compared with the gelatin-only patches, while their elongation potential matched that of the PCL-only patches.

3.3 Morphology and Viability of hiPSC-CMs on Aligned Coaxial Nanofibrous Patches

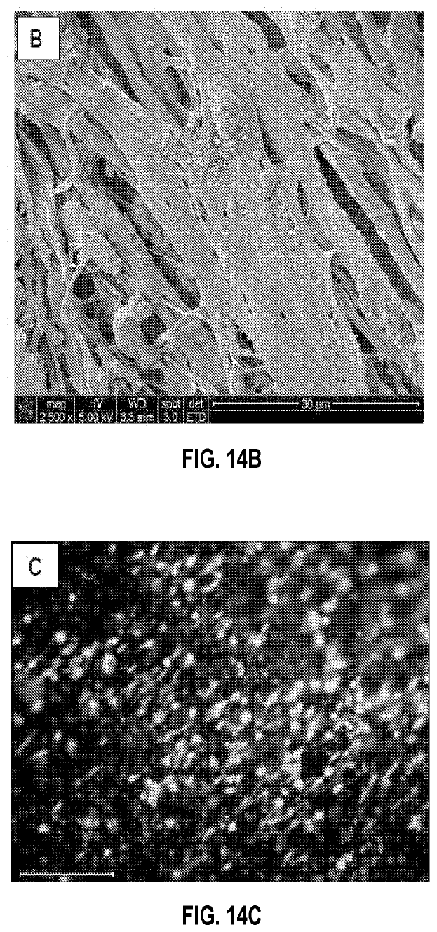

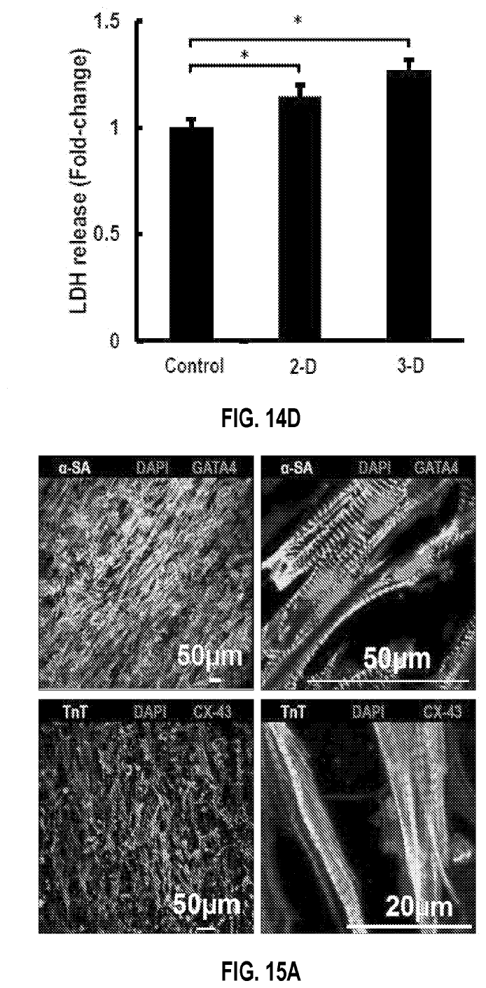

The hiPSC-CMs cultured on aligned coaxial patches and their morphology was assessed at two weeks by SEM. The SEM images showed a uniform distribution and attachment of the hiPSC-CMs on the coaxial patches at two weeks (FIG. 14A). Also, the hiPSC-CMs showed a parallel alignment with the nanofibers (FIG. 14B). The viability of the hiPSC-CMs cultured on the coaxial patches was assessed by staining with Calcein-AM and performing an LDH assay. Fluorescence images showed that the cells were viable and metabolically active, as they stained positive for calcein-AM cells (FIG. 14C). Additionally, the calcein-AM staining showed that the hiPSC-CMs had an aligned morphology on the coaxial patch, which reiterated the SEM findings. Furthermore, LDH assay showed that the level of LDH released from hiPSC-CMs cultured on the aligned coaxial nanofibrous patches (3-D) was not significantly different from hiPSC-CMs cultured on a flat-plate (2-D) (FIG. 14D). Taken together, these results showed that aligned coaxial patches can be used for the culture of hiPSC-CMs, since they do not induce a toxic effect on cell viability and metabolism. These aligned coaxial PCL/Gel patches are biocompatible and support the 3-D culture of hiPSC-CMs.

3.4 Assessment of Cardiac Marker Expression in hiPSC-CMs Cultured on Aligned Coaxial Patch

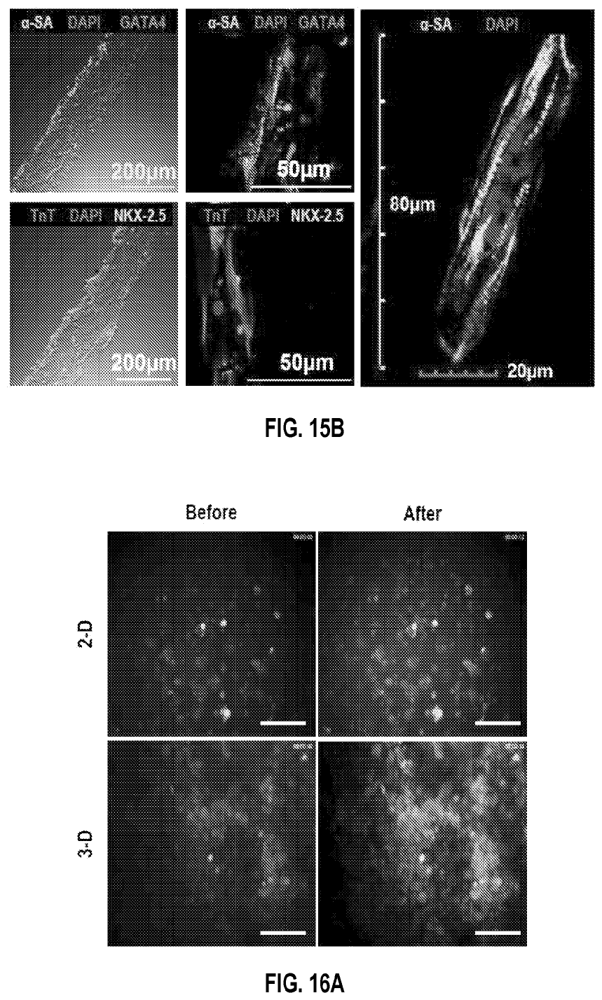

The expression of cardiac markers in hiPSC-CMs was assessed by immunofluorescence studies to understand the intracellular sarcomere arrangement in the cells following culture on the aligned coaxial patches. At two weeks, the hiPSC-CMs cultured on aligned coaxial patches showed they maintained the expression of the cardiac lineage marker, GATA 4 (FIG. 15A). Additionally, these hiPSC-CMs showed parallelly arranged sarcomeres, as observed by α-sarcomeric actinin (α-SA) and cardiac Troponin T (TnT) staining (FIG. 15A). Also, the hiPSC-CMs showed the expression of connexin-43 (Cx-43), indicating good intracellular contact between neighboring cardiomyocytes (FIG. 15A). The distribution of the hiPSC-CMs was also assessed across the depth of the patch. The cross-sections of aligned coaxial cardiac patches showed that the hiPSC-CMs were present up to 40-50 microns below the surface (FIG. 15B), evidenced by the expression of α-SA, TnT, Cx43, and NKX2.5. Additionally, increased magnification of a single hiPSC-CM imaged on the patch showed a multi-nucleated rod-shaped morphology with well-organized sarcomeres (FIG. 15B). These observations indicated that the aligned coaxial nanofibrous patch can be used as a model for understanding the structural maturation of hiPSC-CMs on 3-D scaffolds.

3.5 Assessment of Calcium Transients in hiPSC-CMs Seeded on an Aligned Coaxial Cardiac Patch

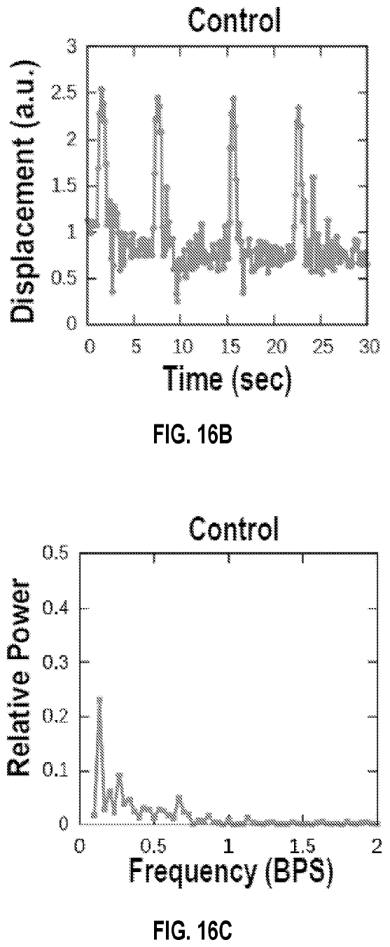

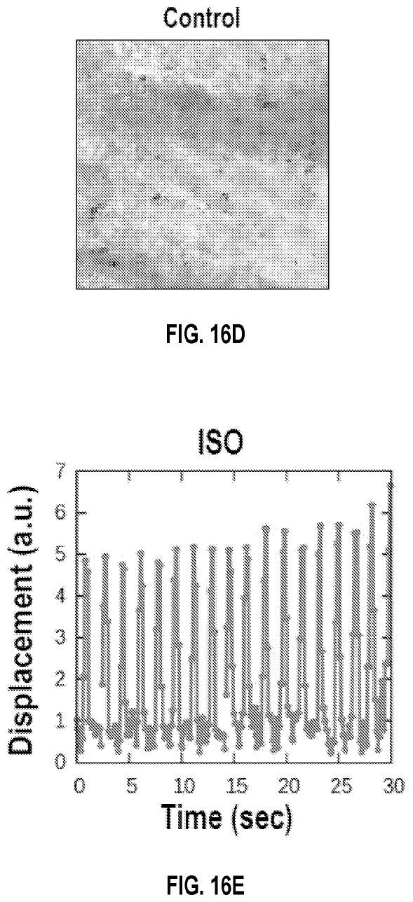

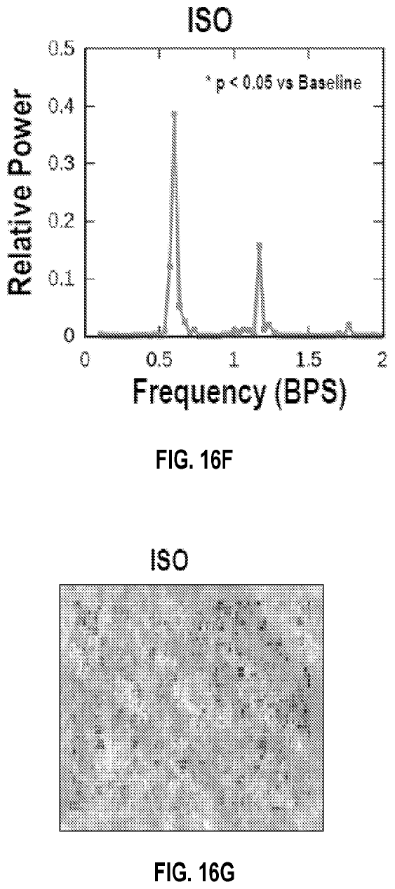

The calcium transients in hiPSC-CMs cultured on tissue culture plates (2-D) and aligned coaxial patches (3-D) were assessed after two weeks in culture. The hiPSC-CMs cultured in 2-D and 3-D showed synchronous calcium transients, indicating the formation of a syncytium between neighboring cells (FIG. 16A). This data showed that the hiPSC-CMs cultured on aligned coaxial patches formed a functional syncytium as indicated by synchronous calcium waves.

3.6 Assessment of Cell Contractility in hiPSC-CMs Cultured on Aligned Coaxial Patches

PIV analysis was performed to evaluate the contractility of hiPSC-CMs cultured on aligned coaxial patches. For this, the response of the patches, at two weeks of culture, to 100 nM isoproterenol (ISO), a non-specific β-adrenoreceptor agonist, was analyzed by an optical measure of contractility (FIGS. 16B-16G). For both treated and control cultures, six videomicroscopic recordings were obtained from two parallel cultures. Beat patterns (FIGS. 16B and 16E) indicated an isoproterenol-induced increase in spontaneous beat frequency and amplitude. Also, Fourier power spectra of the beat patterns indicated an increase in frequency by shifting the dominant peak towards higher frequencies (right) after ISO treatment (FIGS. 16C and 16F). A significant increase (p<0.0004) in the average frequency of spontaneous beating from 0.136+−0.0069 SEM Hz to 1.497+−0.0394 SEM Hz was observed following ISO treatment (FIGS. 16C and 16F). Additionally, ISO treatment also promoted better-defined beating activity that is indicated by the tall narrow peaks. The contractility maps showed a stronger, spatially extended beating activity in ISO-treated patches (FIGS. 16D and 16G). These results demonstrate the ability of the aligned coaxial patches to respond to cardiac drugs in a reproducible manner.

3.7 Electrophysiological Assessment of hiPSC-CMs Cultured on Aligned Coaxial Patches

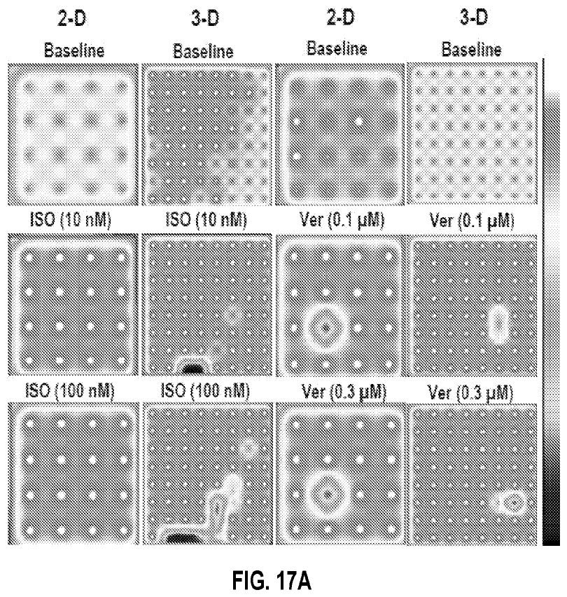

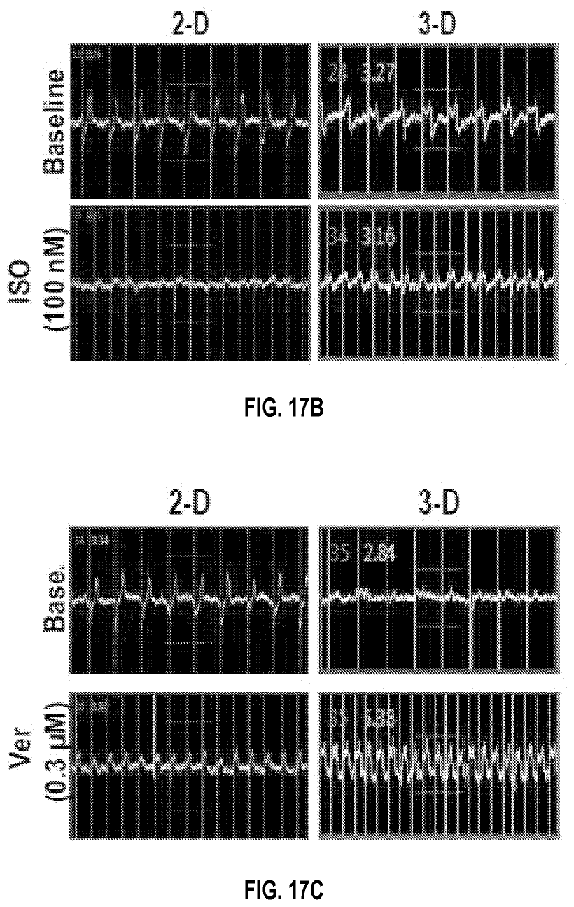

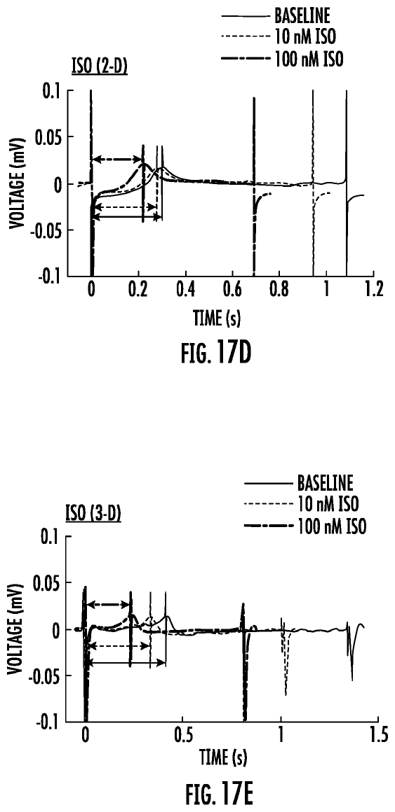

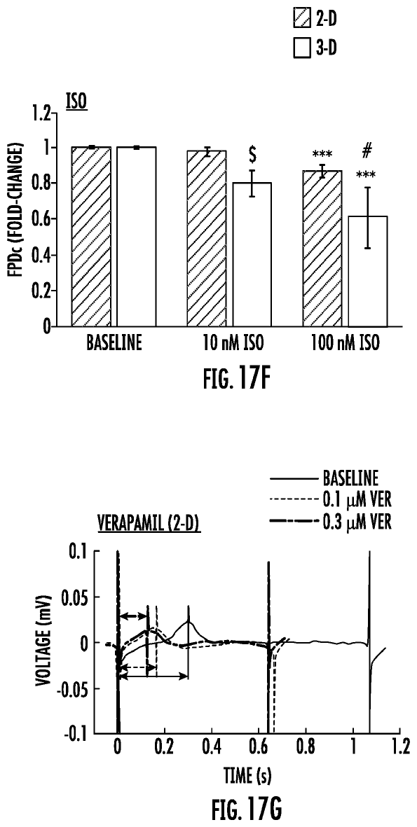

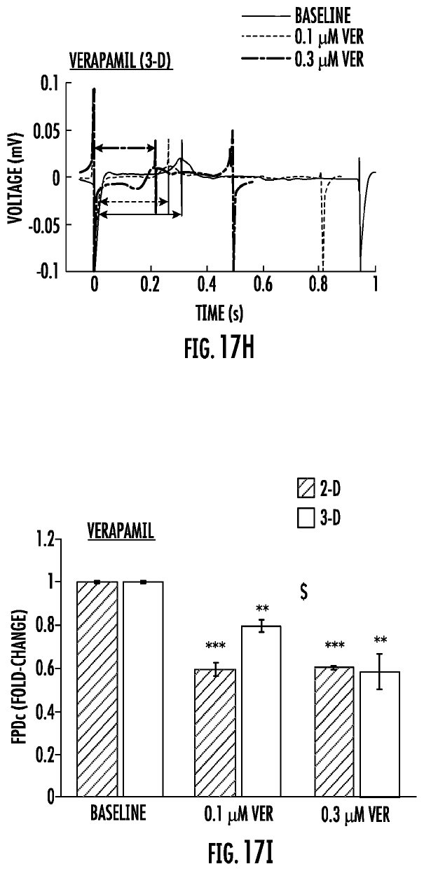

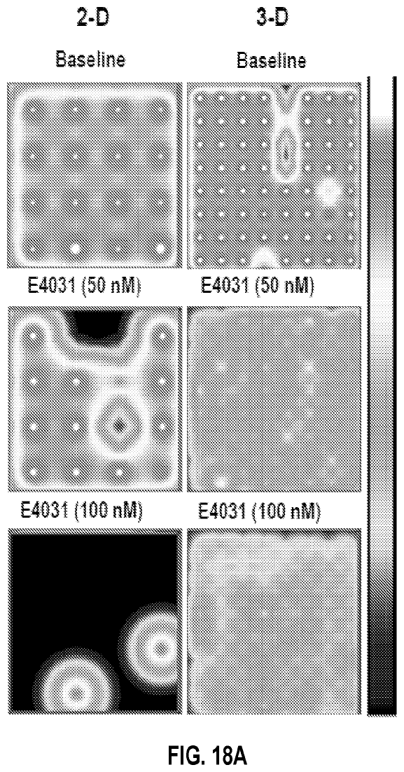

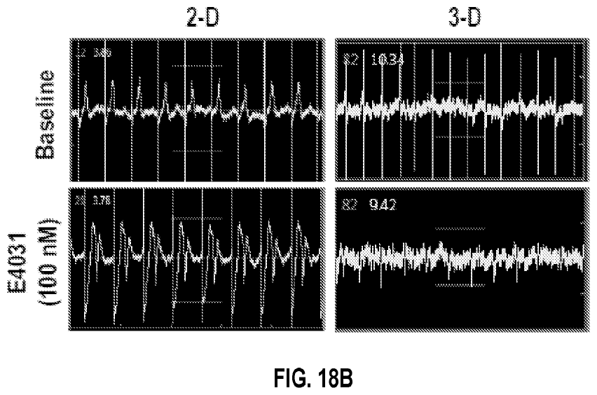

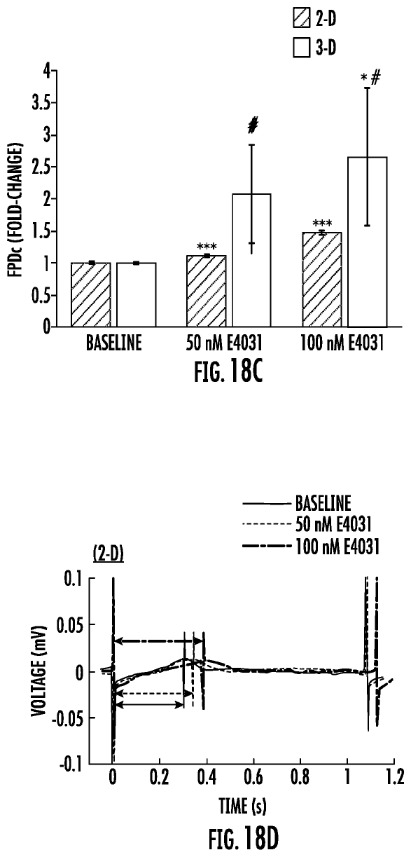

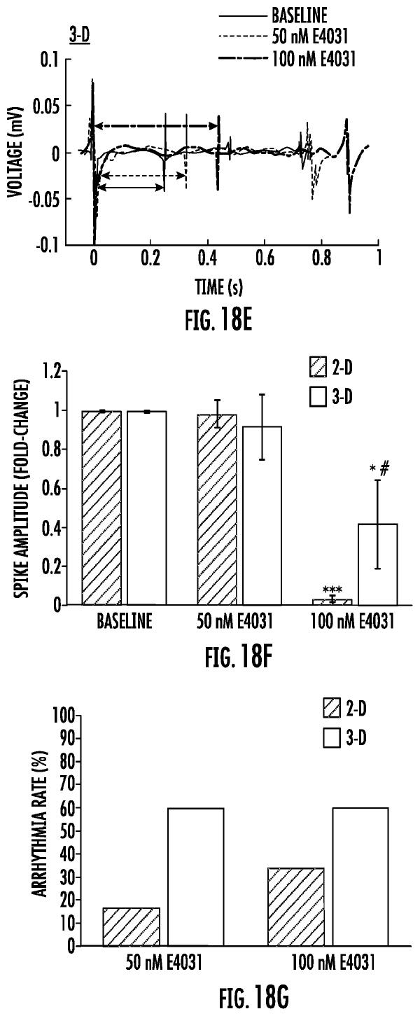

The hiPSC-CMs cultured in 2-D and 3-D (aligned coaxial patch) was assessed on the MEA system for field potentials, which result from spontaneous cardiac action potentials propagating across cells on neighboring electrodes. The hiPSC-CMs were treated with different concentrations of ISO, Verapamil, and E4031, and the changes in their field potential was measured. An increase in the beating frequency (beats per minute) was observed in both the 2-D as well as 3-D cultures following treatment with ISO (FIGS. 17A and 17B) and verapamil (FIGS. 17A and 17C), while a decrease in beating frequency was observed following E4031 treatment (FIGS. 18A and 18B).

A significant dose-dependent decrease in the beat period was observed in hiPSC-CMs in both 2-D and 3-D cultures after treatment with ISO. After treatment with 10 nM ISO, the beat period in the 2-D and 3-D cultures decreased from 1.04±0.04 seconds and 1.22±0.16 seconds to 0.67±0.04 seconds and 0.8±0.15 seconds, respectively (FIGS. 17D and 17E). Similarly, after treatment with 100 nM ISO, the beat period of the hiPSC-CMs in 2-D and 3-D cultures was 0.66±0.03 seconds and 0.66±0.1 seconds, respectively (FIGS. 17D and 17E) indicating that the dose-dependent response was improved in case of 3-D cultures. Furthermore, ISO treatment resulted in a dose-dependent shortening of the QT interval, as evidenced by the decrease in Friedrica's corrected field potential duration (FPDc) in both the 2-D and 3-D cultures (FIGS. 17D-17F). Also, between the 2-D and 3-D cultures, a significant difference in the FPDc was observed after treatment with 10 and 100 nM ISO, indicating that the 3-D cultures responded better to the treatment (FIG. 17F).

A similar decrease in the beat period and FPDc was observed in the hiPSC-CMs cultured in 2-D and 3-D cultures after treatment with the L-type Ca channel blocker Verapamil (FIGS. 17C and 17G-17I). A significant fold decrease in the beat period was observed in both the 2-D as well as 3-D cultures after treatment with 0.1 μM and 0.3 μM verapamil, as compared to their respective baseline controls (FIG. 17I). However, a dose-dependent decrease in the FPDc was observed only in the case of the 3-D culture indicating that the 3-D culture system was a better model for drug testing (FIG. 17I).

The effect of the hERG-type K⁺ channel blocker, E-4031, was also assessed. It is an anti-arrhythmic drug. However, this drug is pro-arrhythmic in vitro especially at higher doses. Following treatment with the E4031, a significant increase in the beat period and FPDc was observed in the 2-D cultures following treatment with 50 nM and 100 nM of E-4031, while a significant increase was observed in 3-D cultures only after treatment with 100 nM E-4031 (FIGS. 18C-18E). Furthermore, higher variability in the FPDc was observed in 3-D cultures as compared to the 2-D cultures (FIG. 18C). This was reiterated by a Comprehensive in vitro pro-arrhythmia assay (CiPA), which showed that 16.67% and 33% of the samples in 2-D cultures showed beat period irregularity after treatment with 50 nM and 100 nM E-4031, respectively, while 60% patches showed beat period irregularity following treatment with E-4031. This data indicated the occurrence of arrhythmias in these cardiac patches (FIG. 18G). Additionally, a significant decrease in the spike amplitude was observed in both the 2-D and 3-D cultures, following treatment with 100 nM E-4031 (FIG. 18F). Taken together, these results showed that hiPSC-CMs cultured in 2-D and 3-D cultures responded to drug treatments and that 3-D cultured hiPSC-CMs can be a better model for in vitro drug testing.

The main focus of the present study was to develop an efficient 3-D culture system for cardiac drug testing. The findings shown herein demonstrate that aligned PCL/Gel coaxial nanofibrous scaffolds successfully culture hiPSC-CMs in a 3-D microenvironment. The comparative assessment of hiPSC-CMs cultured in 2-D and 3-D culture systems carried out in the present study showed improved functional characteristics and increased responsiveness to cardiac drugs in the latter.

Coaxial PCL-gelatin scaffolds demonstrated excellent biological properties including cell attachment, organization, and expression of cardiac lineage markers along with high mechanical strength and modulus allowing for the scaffold to be handled easily. These coaxial scaffolds demonstrated good biomimetic properties similar to gelatin scaffolds, in addition to the high mechanical strength found in PCL scaffolds. These observations show coaxial electrospinning as an efficient strategy for surface modification of nanofibrous scaffolds made from synthetic polymers like PCL, PLGA, and PVA. While coaxial electrospinning has been more commonly used for controlled drug/biomolecule release, it has also been used to coat synthetic polymer-based nanofibers with a natural polymer (like gelatin, alginate, and collagen) to make them more biomimetic. The use of coaxial nanofibers with a PCL inner core and gelatin outer shell has previously been used for wound healing and vascular and bone tissue engineering applications. This coaxial structure improves the biocompatibility of the scaffolds as well as provide structural support to the cells for in vivo applications.

Another striking observation made in this study was the alignment of hiPSC-CMs with the nanofibers in the scaffold. As a result, the hiPSC-CMs cultured on the aligned scaffolds showed an elongated rod-shaped morphology. Additionally, the sarcomeres in these hiPSC-CMs showed parallel organization inside the cell, with some cells being binucleated. These observations indicate the maturation of the cells cultured on the coaxial scaffolds. These observations are consistent with previous reports demonstrating a similar alignment of hiPCMs when cultured on aligned scaffolds. These studies have clearly shown enhanced maturation of hiPSC-CMs cultured on aligned 3-D scaffolds based on increased expression of cardiac genes, re-organization of sarcomeres, and an adult cardiomyocyte-like rod-shaped morphology of the cells. Additionally, it has been observed that scaffolds with fibrous, aligned structure mimic the structure of heart tissue, thereby providing a 3-D microenvironment for anisotropic arrangement of hiPSC-CMs similar to cardiomyocytes in the heart. Of relevance is another observation made in a previous study, wherein hiPSC-CMs cultured in 2-D showed similar morphological maturation, but only after four weeks in culture. This further reiterates that 3-D cultures are more relevant for structural and functional maturation of hiPSC-CMs when compared to 2-D cultures.

Another important aspect of an in vitro model system for cardiac tissue, is the development of a functional syncytium of beating cardiomyocytes. It is a well-established fact that the electrical interconnectivity of cardiomyocytes is an essential pre-requisite for developing in vitro cardiac tissues for drug testing applications. This is especially important to determine the effect of a drug molecule on the heart function (e.g. heart rate, arrhythmia-inducing potential). Hence, cell-cell interaction between cardiomyocytes is extremely critical. Previous studies have shown the formation of a functional syncytium in 2-D cultures mainly due to hypertrophic growth of hiPSC-CMs. On the contrary, functional coupling between cardiomyocytes cultured on 3-D constructs has been reported only after mechanical or electrical stimulation. The current study observes the formation of a functional syncytium of hiPSC-CMs cultured on the coaxial patches, evidenced by the expression of the gap junction protein, Cx-43 and synchronous calcium transients across the patch. These observations are similar to the behavior of hiPSC-CMs cultured in 2-D, further indicating that the aligned coaxial scaffolds can be used as an in vitro culture system.

It has been shown that 3-D culture systems making use of hiPSC-CMs are better suited for cardiac drug testing and toxicity studies because they mimic the in vivo response. However, the use of 3-D cultures has been restrained due to lack of (a) extensive literature, (b) reproducible culture protocols, and (c) precise analysis tools. In the recent years, several different techniques have been developed and optimized for analyzing the effect of different cardiac drugs on hiPSC-CMs in 3-D cultures, such as calcium imaging using fluorescent dyes, PIV, and measurement of field potentials by MEA. In the study shown herein, these analysis tools were used to monitor the effectiveness of a few commonly used cardiac drugs on hiPSC-CMs cultured in 2-D vis-à-vis 3-D cultures. An increase in the beat rate along with a decrease in beat period and FPDc was observed in both the 2-D as well as the 3-D cultures after ISO treatment. These observations are consistent with previous studies, where a dose-dependent increase in contractility was observed in 2-D hiPSC-CMs following treatment with ISO, as a result of $1 and 02 receptor stimulation. Additionally, although verapamil has been known to inhibit voltage-dependent calcium channels, and increased contractility was observed in both 2-D and 3-D cultures.

However, consistent with the observation shown herein. a recent study identified that this discrepancy in the effect of verapamil in vitro and in vivo, resulted from the low potassium concentration in cell culture medium. On the other hand, E-4031, an hERG channel blocker, resulted in prolonged QT interval (FPDc) in addition to a decrease in the spike amplitude in a dose-dependent manner for 3-D cultured cells. The incidence of arrhythmicity was also high following the addition of E-4031 in both the 2-D as well as 3-D cultures. Furthermore, 3-D cultures showed four times greater response to drugs than 2-D cultures. These observations are in agreement with earlier reports using hiPSC-CMs. Overall, these results strongly indicated an elevated sensitivity of hiPSC-CMs in 3-D cultures to various drugs used in this study. Similar observations were reported in the case of primary hepatocytes and cancer cells. Likewise, a recent study demonstrated that 3-D cardiac spheroids mimics an in vivo milieu drug testing and cardiotoxicity studies.

Overall, this study demonstrated the fabrication of an aligned coaxial PCL-gelatin nanofiber patch with mechanical and biomimetic properties desired for cardiac applications. hiPSC-CMs cultured on these patches showed a rod-shaped morphology and were aligned in parallel with the nanofibers. These cells on the cardiac patch showed synchronous contractions and exhibited quick response to cardiac drugs. Finally, these cardiac patches can be used as an in vitro drug screening platform for cardiotoxicity studies, as well as to develop future strategies of cardiac patch transplantation for ischemic heart disease.

### Example 4. Electrospun Aligned Co-Axial Nanofibrous Scaffold for Cardiac Repair

Cardiovascular diseases (CVDs) are one of the leading causes of mortality worldwide and a number one killer in the US. Cell-based approaches to treat CVDs have only shown modest improvement due to poor survival, retention and engraftment of the transplanted cells in the ischemic myocardium. Recently, tissue-engineering and the use of 3D scaffolds for culturing and delivering stem cells for ischemic heart disease is gaining rapid potential. Describe herein is a protocol for the fabrication of aligned co-axial nanofibrous scaffold comprising of a polycaprolactone (PCL) core and gelatin shell. Furthermore, a detailed protocol is shown for the efficient seeding and maintenance of human induced pluripotent stem cell derived cardiomyocytes (hiPSC-CMs) on these nanofibrous scaffolds, which can have an application in the generation of functional ‘cardiac patch’ for myocardial repair applications as well as an in vitro 3D cardiac tissue model to evaluate the efficacy of cardiovascular drugs and cardiac toxicities.

### 1. Introduction

Cell-based approaches have become an integral part in the field of regenerative medicine, especially for organs with limited or no regenerative potential, e.g. the heart. Although a number of different cell types, including various stem/progenitor cells as well as cardiomyocytes have been studied, only a modest improvement in cardiac function has been reported following transplantation into the ischemic myocardium. A vast majority of the studies have attributed this to poor retention and low survival of the cells at the site of infarction. To overcome these limitations, tissue-engineering techniques have been employed in combination with cells to improve the ease of cell delivery as well as retention. Among these techniques, use of 3D bioengineered scaffolds in combination with different cell types has become increasingly popular. Such scaffolds have been successfully used in various pre-clinical as well as clinical studies. However, several factors have been shown to influence the successful use of such bioengineered scaffolds for cardiac applications, namely, the nature of biomaterial used, mechanical properties of the scaffolds, thickness and dimensions, and alignment.

Studies have reported the use of natural biomaterials such as collagen, gelatin and fibrin in scaffolds for culturing cardiomyocytes. While these materials do not alter the cell viability or function, they have been shown to exhibit poor strength and stiffness. On the other hand, synthetic polymers like polycaprolactone (PCL) and poly (lactic-co-glycolic acid) (PLGA) have been used to fabricate scaffolds with better mechanical properties, but the adhesion and survival of cells on these patches was low. Co-axial (Co-A) nanofiber systems, with a synthetic polymer (PCL or PLA) in the core and a more adhesive natural material (gelatin) as the shell, conferred both mechanical strength, high cell adhesion and viability, and lower in vitro production of inflammatory cytokines when compared to purely synthetic nanofibrous scaffold. For tissue-engineering applications, among various available techniques, electrospinning has been extensively used for fabricating nanofibrous scaffolds. In this invention, a method is shown for the fabrication of an aligned polycaprolactone (PCL)-gelatin co-axial nanofibrous scaffold for the development of cardiac patch, which can be implanted along with hiPSC-CMs on the epicardial heart surface for cardiac tissue regeneration.

### 2. Materials

Prepare all reagents using sterile ultrapure water and culture grade reagents. Prepare and store all reagents at room temperature (until mentioned otherwise). Prepare all organic solvents in a chemical hood and use the appropriate personal protective equipment.

### 2.1. Electrospinning:

1. 12 wt./vol. % Gelatin solution: Dissolve 1.2 g of Gelatin from Bovine Skin in 10 ml of 1,1,1,3,3,3-hexafluoro-2-propanol (HFIP) (see Note 1).
2. 8 wt./vol. % Polycaprolactone (PCL): Dissolve 0.4 g of PCL in c5 mL 1,1,1,3,3,3-hexafluoro-2-propanol (HFIP) (see Note 1).
3. 18 gauge needle
4. T-shaped spinneret tube
5. Syringe pumps
6. Rotating collector

### 2.2. Cross-Linking

1. 7 mM N-(3-Dimethylaminopropyl)-N′ ethylcarbodiimide hydrochloride (EDC) solution (Cross-linking solution): Dissolve EDC in sterile 100% ethanol
2. Sterile 70% ethanol
3. Sterile Phosphate-buffered saline (PBS)
4. Biopsy punch (8 mm)
5. Sterile forceps
6. 94 mm dishes
   2.3. Culture of hiPSC-CMs
1. hiPSC-CMs (Fujifilm Cellular Dynamics, WI): Thaw and culture the hiPSC-CMs as per the manufacturer's instructions on 1% gelatin-coated 6 well plates [5].
2. 50 μg/ml Fibronectin solution: Dissolve 50 μg human fibronectin in 1 ml PBS.
3. iCell cardiomyocyte maintenance medium (CMM, Fujifilm Cellular Dynamics, WI)
4. 0.25% trypsin-EDTA
5. 94 mm tissue culture-treated dishes
6. 6 well plates
7. Sterile sponge (see Note 2).
8. Sterile N-Terface (Winfield Labs; see Note 2).

### 2.4. Autoclaved Forceps and Scissors Scanning Electron Microscopy:

1. 4% paraformaldehyde (PFA) solution: Dissolve 4 g of PFA in 100 ml PBS.
2. 50%, 70%, 80%, 95% and 100% ethanol gradients (in PBS)
3. 25%, 50%, 75% and 100% Hexamethyldisilazane (HDMS) gradients (in ethanol)
4. Carbon tape

### 2.5. Confocal Microscopy:

1. Gelatin-fluorescein solution: Dissolve 1 g of fluorescein in 100 ml of 12% gelatin solution
2. PCL-rhodamine solution: Dissolve 1 g of rhodamine in 100 ml of 8% PCL solution.
3. Glass slides
4. Coverslips
5. Prolong Diamond Antifade

3. Methods

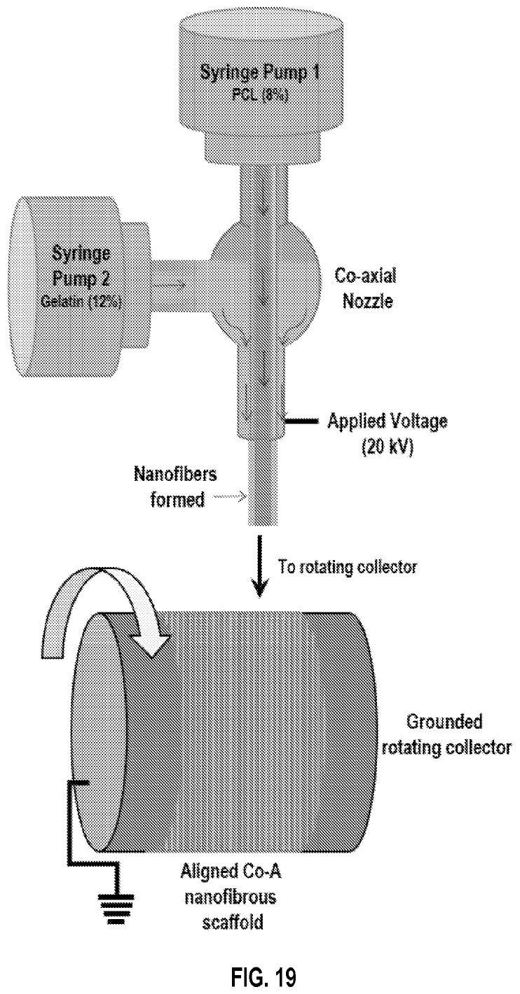

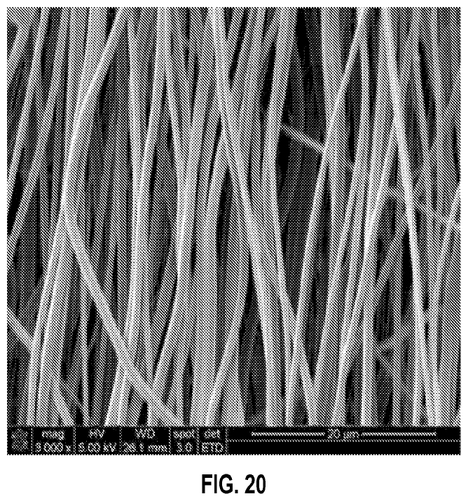

3.1. Scaffold Preparation
1. Setting up a Co-A spinneret: Insert a 18 G needle through a T-shaped spinneret tube to obtain a Co-A spinneret set-up. Manually wrap the 18 G needle with a thin wire to ensure that the inner needle is positioned at the center of the T-shaped spinneret and does not move vertically or laterally (see Note 3, 4). FIG. 19 shows the schematic of the entire electrospinning setup (see Note 5).
2. Set-up the syringe pumps to feed 12 wt./vol. % Gelatin solution and 8 wt./vol. % PCL solution at flow rates of 4 ml/hr and 1 ml/hr, respectively (see Note 6).
3. Adjust the distance between the spinneret nozzle and the grounded rotating collector to 20 cm (see Note 7).
4. Maintain the co-axial spinneret nozzle at a voltage of 20 kV.
5. Collect the aligned co-axial nanofibers which start forming on the rotating collector, when the voltage is applied on the nozzle and the collector grounded.
6. Allow the collected aligned co-axial nanofibers to dry overnight in the chemical hood in order to remove the remnant solvent (see Note 8).

### 3.1.1. Support Protocol 1: Scanning Electron Microscopy of Scaffolds

The alignment of the nanofibers in the scaffold and the uniformity of the fibers can be analyzed by scanning electron microscopy (SEM, FIG. 20)
1. Cut a 0.5 cm×0.5 cm piece of the nanofibrous scaffold
2. Mount the piece onto a carbon tape and subject it to gold-palladium sputter coating as per the instrument manufacturer's instructions
3. Image the coated scaffolds on a scanning electron microscope.

### 3.1.2. Support Protocol 2: Confocal Microscopy of Scaffolds

The co-axial nature of the nanofibers formed can be verified by confocal microscopy.
1. Use gelatin and PCL solutions containing fluorescein and rhodamine, respectively for fabrication of the nanofibers as described above.
2. Cut small pieces of the nanofiber scaffold and follow the cross-linking process as mentioned in section 3.2
3. Place a small piece of the scaffold onto a glass slide.
4. Mount a glass coverslip on the scaffold with 1-2 drops of Prolong Diamond Antifade.
5. Image the nanofibers on a confocal microscope (see Note 9).

### 3.2. Cross-Linking and Sterilization

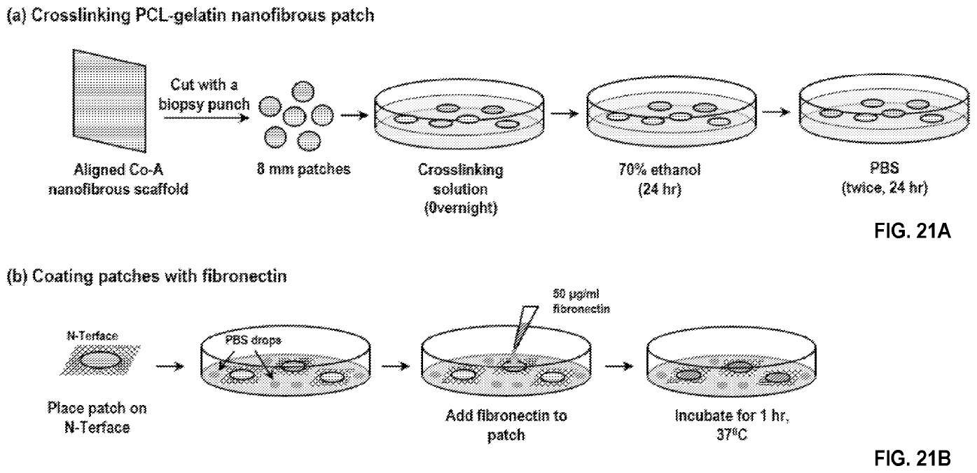

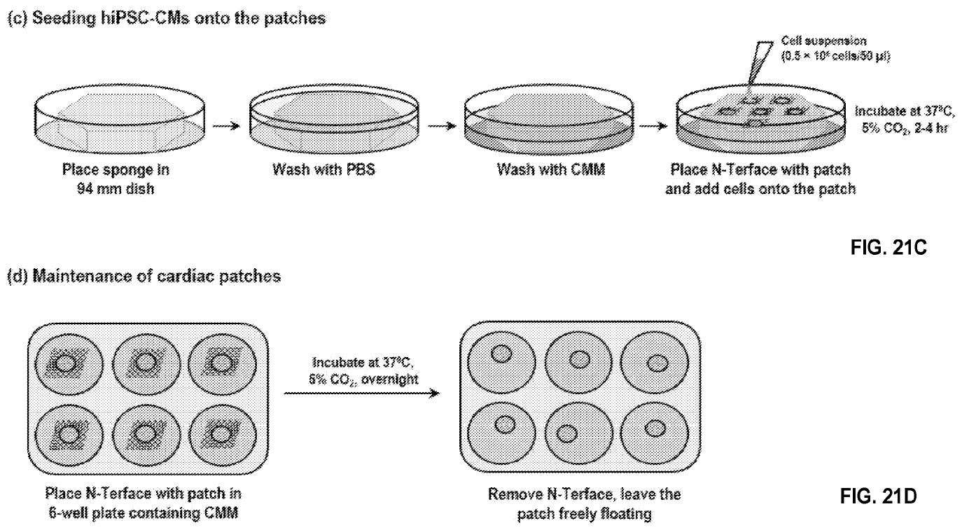

1. Using a biopsy punch, cut the aligned Co-A nanofibrous scaffold into small circular pieces (FIG. 21).
2. Place the scaffold in a 94 mm dish containing cross-linking solution (Make a solution of EDC in 100% ethanol; for gelatin or gelatin-PCL or gelatin-PLA scaffolds use 7 mM and 5 mM for collagen scaffolds) overnight at room temperature.
3. Next day, transfer the scaffold in 70% ethanol in a sterile dish and incubate for 24 hr (Note: the scaffolds will shrink).
4. The next day aspirate the 70% ethanol and add fresh sterile PBS to the scaffold and incubate for 24 hr.
5. Aspirate the PBS and add fresh sterile PBS. Allow dishes to sit overnight.
6. Remove PBS and rinse 5 times with PBS for 20 min each followed by medium. Punch/cut scaffolds to the appropriate size and inoculate/seed cells (Note: DO NOT keep unused scaffolds). They are intended to be crosslinked, rinsed and used immediately. Thus, plan to start the crosslinking process 5 days prior to seeding as described in section 3.3.
   3.3. Seeding hiPSC-CMs on an Aligned Nanofibrous Scaffold

### 3.3.1. Coating of Scaffold

1. Cut out 20 mm×20 mm squares of the sterile N-Terface and place the pieces carefully in a 94 mm dish (FIG. 21).
2. Place one cross-linked 8 mm scaffold on the N-Terface carefully using sterile forceps. (see Note 10).
3. Add 30 μl of fibronectin solution onto each scaffold. Avoid spilling over of the fibronectin out of the scaffold (see Note 11).
4. Incubate the scaffold at 37° C. for at least 1 hr.

### 3.3.2. Seeding Cells on the Scaffold

1. Place a sterile sponge in a 94 mm dish and add 15 ml of PBS over it (FIG. 21). Once the sponge is completely soaked in PBS, aspirate out the PBS. Lightly press the sponge to ensure complete removal of PBS. Repeat this step twice.
2. Repeat step 1 with CMM.
3. Transfer the scaffold along with the N-Terface onto the sponge as shown in FIG. 3.
4. Wash the hiPSC-CMs twice with PBS to remove any traces of medium in it.
5. Add 1 ml of 0.25% trypsin-EDTA (Pre-warmed at 37° C.) per well of a 6-well plate.
6. Incubate the cells at 37° C. for 5-7 min (see Note 12).
7. Add equal volume of CMM to the cells to neutralize trypsin activity. Dislodge the cells from the cell culture plate by gentle pipetting (see Note 13).
8. Collect the cells into a 15 ml centrifuge tube. Centrifuge the cells at 300 g for 10 min at RT.
9. Re-suspend the cell pellet in 5 ml CMM and count the number of the cells.
10. Centrifuge the cells again at 300 g for 10 min at RT and re-suspend the cells in appropriate volume of CMM to obtain 0.5 million cells/50 μl (see Note 14).
11. Add 25 μl of cell suspension onto each scaffold. After 2-5 min, add 25 μl of cell suspension once again onto each scaffold (see Note 15).
12. Carefully place the dish in the incubator at 37° C., 5% CO₂ for 4-6 hrs.
13. At the end of incubation, carefully pick the N-Terface along with the scaffold using forceps and transfer them into a 6-well plate containing 2 ml of CMM/well. (see Note 16).
14. Incubate the scaffold at 37° C., 5% CO₂ overnight.
15. The following day, carefully remove the N-Terface from under the scaffold without disturbing the orientation of the scaffold (see Note 17, FIG. 21).
16. Add an additional 1 ml of CMM per well of the 6-well plate.
17. Change the medium for the scaffold after every 48 hrs.

### 3.3.2.1. Support Protocol 3: Scanning Electron Microscopy of Cells:

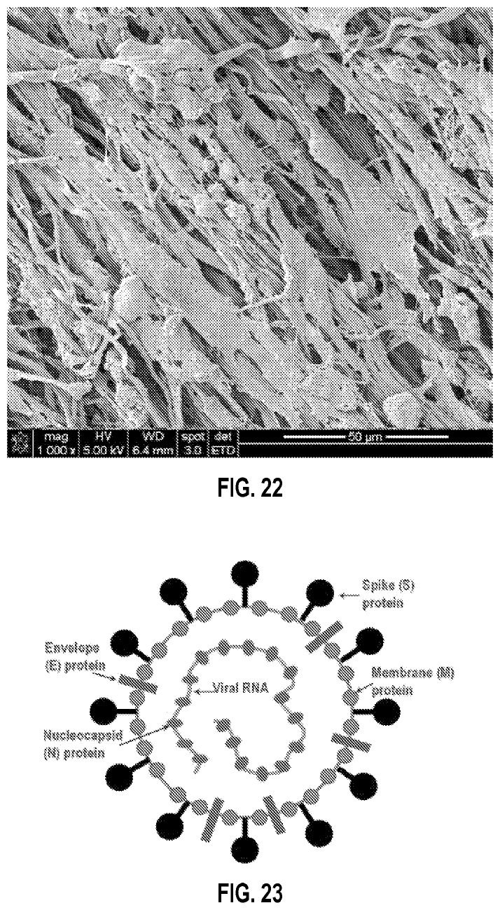

The hiPSC-CMs seeded onto the scaffold can be imaged using a scanning electron microscopy (SEM, FIG. 22)
1. Wash the hiPSC-CMs cultured on the aligned nanofibrous scaffold with PBS (2 times)
2. Fix the scaffold in 4% PFA for 20 min at RT
3. Wash the scaffold twice in PBS
4. Gradually dehydrate the scaffold using 50%, 70%, 80%, 95% and 100% ethanol gradients. Incubate the scaffold for 20 min in each gradient solution.
5. Gradually dry the scaffold using 25%, 50%, 75% and 100% HDMS gradients. Incubate the scaffold for 10 min in each solution. After incubation in 100% HDMS for 10 mins, remove HDMS and allow it to evaporate completely in the hood before cutting the scaffold.
6. Make a cut on the scaffold (approximately 75% of the diameter).
7. Mount the scaffold onto the carbon tape and fold the scaffold along the slit such that both the sides of the scaffold are exposed on the top for sputter coating.
8. Subject the scaffold to gold-palladium sputter coating as per the instrument manufacturer's instructions
9. Image the coated scaffolds on a scanning electron microscope.

### 4. Notes

1. Cover the beaker to prevent evaporation of the solvent. Place the solution on a magnetic stirrer to ensure complete dissolution. Leave the PCL and gelatin solutions for 2 hr and 48 hr, respectively for complete dissolution.
2. Sterilize the sponge and N-Terface using ethylene oxide gas or gamma irradiation prior to use in cell culture.
3. Make sure the 18 G needle (the inner tube) and the T-shaped spinneret tube (the outer one) are snugly fit and is not moving. This is very crucial to obtain co-axial nanofibers.
4. The inner needle acts as tube which delivers the core (PCL) solution while the outer tube delivers the shell (Gelatin solution).
5. The electrospinning process is very sensitive to humidity. Make sure to maintain the humidity of the setup between 35-40% relative humidity.
6. While using the syringe pump, make sure to select the appropriate syringe diameter and the flow rate.
7. Make sure the Co-A nozzle is aligned in line with the rotating collector to maximize the nanofiber collection yield.
8. Make sure to remove all the remnant solvent by keeping the scaffold inside the chemical hood for at least for 24 hours
9. Ensure crosslinking is performed before confocal imaging to stabilize the gelatin shell. Gelatin will degrade quickly without crosslinking.
10. Ensure that the scaffold lies flat on the N-Terface and no crease appears on it to prevent improper coating and/or distribution of cells on the scaffold. In case the scaffold folds while placing, gently straighten it using blunt forceps. Be careful to avoid penetrating/tearing the scaffold.
11. Allow the scaffold to dry for 1-2 min before addition of fibronectin onto it. This can help retain the fibronectin on the scaffold and not flow out of it.
12. Monitor the cells every 3-4 min to observe the changes in their morphology. The duration of trypsinization can be extended till the cells start to detach from each other as well as from the dish. Gently rock the dish back and forth every few min to dislodge the cells. Do not tap the dish!
13. Avoid over pipetting.
14. The cell density to be plated is 1 million/cm². If scaffolds of different sizes are to be used, the cell numbers must be varied accordingly.
15. Add the medium dropwise onto the scaffold. Make sure the cell suspension is added evenly throughout the scaffold and not concentrated in one region. Add the second 25 μl after the first medium drop has been completely absorbed by the sponge. Otherwise the cells can spread out of the scaffold.
16. Place the N-Terface such that it floats on the surface of the medium.
17. When removing the N-Terface, gently push it into the medium. When the scaffold comes off and floats freely, take out the N-Terface. Make sure that the scaffold does not fold, crease or invert while doing so.

### 5. Troubleshooting

1. Low viability of hiPSC-CMs after trypsinization: Loss of cell viability can occur if the cells are over-trypsinized. Neutralize trypsin activity once the cells start to detach from the dish. Minimize the number of times the cells are pipetted to re-suspend the cell pellet.
2. Poor attachment of hiPSC-CMs onto the scaffold: This can occur in case of improper fibronectin coating. Do not allow the fibronectin drop to dry during coating. Additional drops of PBS can be placed in the dish to maintain the humidity in the dish. If the problem persists, increase the concentration of fibronectin used for coating (e.g. 60-70 μg/ml) or leave the scaffolds on the sponge overnight to give more time of attachment. Also, while handling the scaffold make sure that the tip of the forceps does not scrap off cells attached to the scaffold.
3. No contracting (beating) scaffolds: Normally, the contracting scaffolds are observed with 48-72 hrs of seeding the hiPSC-CMs onto the scaffold. In case no contractility is observed:
   a. Ensure that the hiPSC-CMs show a synchronous contractility in gelatin-coated dishes before seeding them onto the scaffold. hiPSC-CMs show some batch-batch variation in the initiation of synchronous contractility. However, the time varies depending on the batch of hiPSC-CMs used.
   b. If SEM analysis shows absence of cells: The drop of cell suspension must be added onto the scaffold. Make sure the drop does not spread onto the sponge.
   c. SEM analysis showed non-uniform distribution of cells: The cells need to distribute uniformly and form a syncytium for the scaffold to contract. During seeding, add the cells dropwise throughout the scaffold. However, if the problem persists, increase the seeding cell density to 1.5-2 million/cm².

### 6. Results

The synthesized aligned co-axial nanofibrous scaffold should show a parallel arrangement with each fiber showing a similar thickness (FIG. 20). Visible contacting cardiac scaffolds should be seen within 2-4 days of seeding the cells and SEM analysis must show CMs aligned in the direction of the of the fibers (FIG. 22).

### Example 5. HiPSC-Derived Cardiomyocytes for Evaluating Hydroxychloroquine

The current global pandemic of COVID-19 caused due to SARS-CoV-2 infection resulted in >3.75 million confirmed cases and >250,000 deaths worldwide. In US alone >1.2 million confirmed cases and >67,000 deaths were reported (May 8, 2020). Patients can be asymptomatic carriers, present with mild symptoms or become severely ill and require intensive care. A meta-analysis of studies published as of Mar. 1, 2020 included 1558 patients and demonstrated that hypertension, diabetes, chronic obstructive pulmonary disease (COPD), cardiovascular disease, and cerebrovascular disease were major risk factors for severity of COVID-19. Another study by Mehra et al suggested that underlying cardiovascular disease is associated with an increased risk of in-hospital death among patients hospitalized with COVID-19. The factors independently associated with an increased risk of in-hospital death in patients >65 years old were coronary artery disease (10.2%), heart failure (15.3%), cardiac arrhythmia (11.5%), COPD, and smoking. The lack of an approved antiviral therapy or vaccine for SARS-CoV-2 has led to repurposing of already approved drugs to treat critically ill patients. The FDA has recently issued an Emergency Use Authorization (EUA) allowing hydroxychloroquine (HCQ) and chloroquine to be used for patients hospitalized with COVID-19. In vitro studies have reported antiviral activity of HCQ against SARS-CoV and SARS-CoV-2. However, the serious cardiotoxicity associated with HCQ treatment in COVID-19 patients with underlying heart disease is not fully understood.

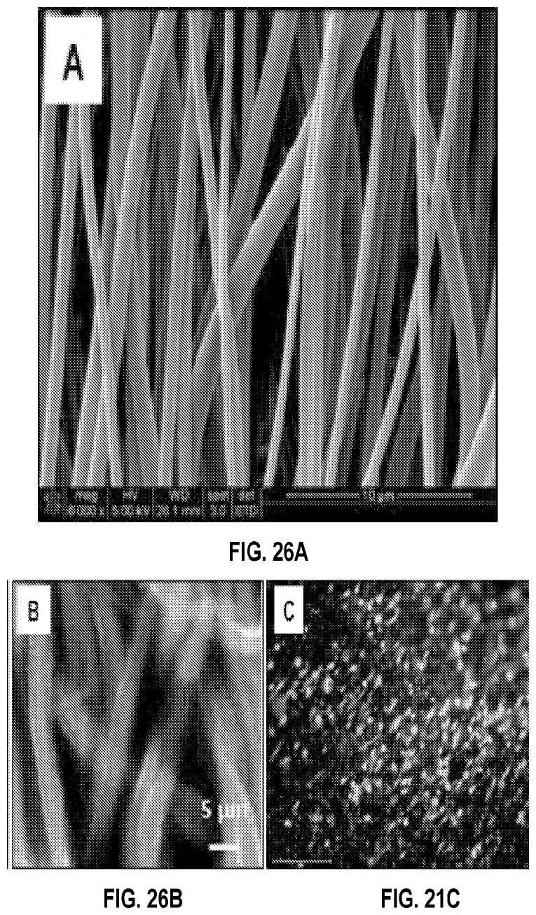

A recent open label non-randomized clinical study was conducted in France to compare treating COVID-19 patients with HCQ and azithromycin (AZ) combination, HCQ alone, or without HCQ. In this study, Gautret et al reported 100% viral clearance in nasopharyngeal swabs in 6 HCQ/AZ-treated patients after 5 and 6 days of inclusion, touting the antiviral efficacy of the treatment. However, of those initially included and treated with HCQ, one patient died and 3 were transferred to the ICU within 4 days and these patients were excluded from analysis. Another study by Molina et al, reported that 11 patients were given the same dose of HCQ/AZ as the Gautret's study, however one patient died within 5 days and 2 patients were transferred to the ICU. Treatment was discontinued after 4 days in one patient due to observed prolongation of the QT interval. Furthermore, a study in New York found no significant association between HCQ treatment and consequent intubation or death (Geleris et al, PMID 32379955). While HCQ is likely to be safe in a majority of the population, it poses serious and potentially lethal risks to patients with underlying heart conditions, like dilated cardiomyopathy (DCM) and heart failure. The mechanism of cardiotoxicity remains still unknown, which leads to clinical uncertainty for who might benefit from this treatment. Hence, understanding the molecular mechanism for cardiotoxicity induced by HCQ treatment of COVID-19 is an urgent need. Therefore, the present study assesses the cardiotoxic mechanism(s) of HCQ treatment in healthy and diseased (DCM) hiPSC-derived cardiomyocytes (hiCMs). Additionally, the well-established 3-D biomimetic model are used (FIGS. 26 and 8), comprised of an electrospun co-axial (PCL core, gelatin shell) aligned nanofiber scaffold seeded with hiCMs, for in situ drug testing. Furthermore, challenging cells with a BSL2-amenable retroviral vector pseudotyped with SARS-CoV-2 Spike protein safely mimics viral attachment and entry, which circumvents BSL-3 safety requirements of live SARS-CoV-2. The use of diseased hiCMs illustrates differences in response for patients with underlying cardiovascular disease, who are at greater risk for adverse side effects of HCQ. This study proposal assesses HCQ-induced cardiotoxicity in hiCMs derived from healthy and dilated cardiomyopathy (DCM) patients in a 3-D cardiac scaffold.

Evaluate the functional effect of HCQ on hiCMs derived from healthy and diseased patients on a 3D scaffold in vitro. To determine the cytotoxicity and functional role of HCQ on cardiomyocytes, experiments are performed for assessing whether HCQ induces changes in electrophysiological function via multi electrode array, patch clamp, mitochondrial function & bioenergetics, and cytotoxicity in hiCMs with/without transduction with virus pseudotyped with SARS-CoV-2 Spike protein. HiCMs derived from healthy and DCM patients are used on a 3-D scaffold as biomimetic cardiac models of healthy patients and those with underlying disease.

Identify molecular mechanisms responsible for HCQ-induced cardiotoxicity in hiCMs in vitro. The molecular mechanisms of HCQ-induced cardiotoxicity are not well understood. Experiments are performed for assessing whether HCQ induces changes in cardiac genes, thereby impacting cardiomyocyte structure. To have a comprehensive understanding of the mechanisms underlying HCQ mediated cardio toxicity, cytokine and cardiac Troponin-I secretion, miRNA profiling, and RNA sequencing are analyzed in 3-D cardiac patches with/without transduction with SARS-CoV-2 pseudotyped virus.

Myocardial injury has been observed with COVID-19 infection as evidenced by elevated serum troponin levels, with mortality associated with coincident functional abnormalities detected by EKG and echo. For a cohort of patients in Wuhan, heart failure contributed to 40% of deaths, either alone or with respiratory failure. Acute cardiac injury had a risk association more significant than age, type 2 diabetes, chronic lung disease, or prior heart disease for severely ill patients. The mechanism behind this damage is unknown but can be due to direct myocardial infection and damage, stress from hypoxemia due to respiratory failure, microvascular damage, or the systemic inflammatory response, or any combination the above. The structure of SARS-CoV-2 has been elucidated by various groups. The virus has been shown to comprise four major proteins in addition to its single positive-stranded RNA genome: (i) membrane (M) protein, which determines the shape of the virus; (ii) envelope (E) protein, which plays a role in maturation of the virus; (iii) spike (S) protein, which facilitates entry of the virus into the host cell; and (iv) nucleocapsid (N) protein, which regulates viral replication and host response to the virus (FIG. 23). The binding of SARS-CoV-2 S-protein to ACE2 facilitates entry of the virus into host cells and due to high expression of ACE2 in the heart, which is increased in failing hearts and DCM patients, can cause failure due to direct cellular damage by the virus.

Additionally, exaggerated systemic inflammation can create a “cytokine release syndrome”, which can induce acute respiratory distress syndrome, and was the major cause of morbidity in SARS-CoV and MERS-CoV patients. COVID-19 has been shown to induce expression of pro-inflammatory cytokines (IL-6, IL-10, IL-2, and IFNγ) in severe cases as compared to mild cases in a Beijing study. Another study in China compared a panel of plasma levels of 48 cytokines in critical, severe, and moderate cases of hospitalized COVID-19 patients. Results determined expression of 14 cytokines increased with COVID-19 disease as compared to healthy controls and at different levels according to disease severity. Critically ill patients had the highest levels of IP-10, MCP-3, HGF, MIG, and MIP-1α, followed by the levels in severely ill then moderately ill patients.

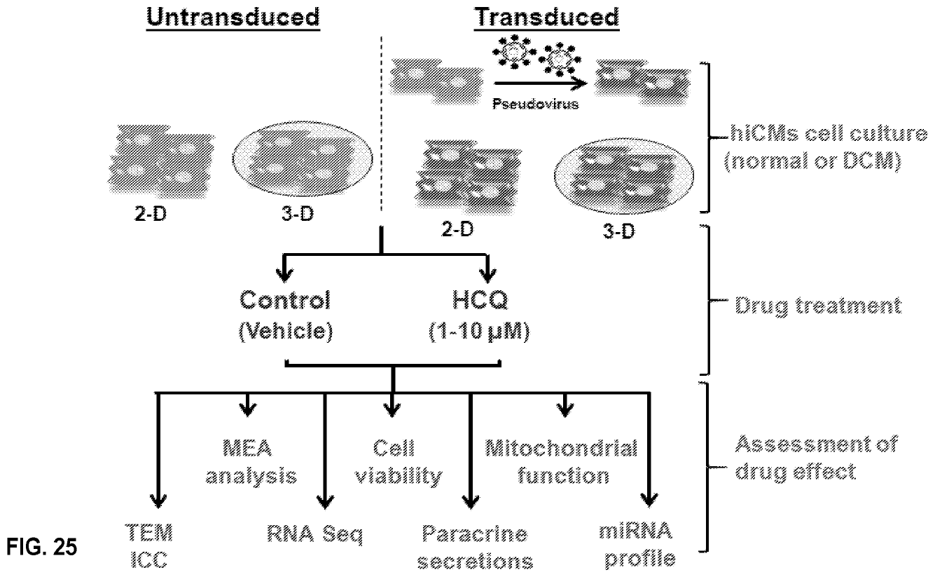

Chloroquine and related drugs were initially developed as antimalarial agents and due to the serendipitous observations made by clinicians these drugs were used for treating rheumatological and dermatological conditions. A recent study found that HCQ was more potent than chloroquine as the effective concentration for a half-maximal response (EC₅₀) was much lower (0.72 μM) for HCQ than for chloroquine (5.47 IM). Hydroxychloroquine has been reported antiviral activity of HCQ against SARS-CoV and SARS-CoV-2. Due to the lack of an approved antiviral therapy or vaccine for SARS-CoV-2, HCQ is currently being used in critically ill patients hospitalized with COVID-19. However, the potential serious cardiotoxicity associated with HCQ treatment in COVID-19 patients with underlying heart disease is not fully understood. Therefore, the present investigates electrophysiological function, cardiotoxicity, ultrastructural morphology and RNA sequencing in hiCMs in vitro, in both healthy and diseased cardiomyocytes treated with/without HCQ/SARS-CoV-2 pseudotyped virus transduction as shown in FIG. 25.

In the midst of the COVID-19 pandemic, there is an urgent need for effective antiviral therapies and HCQ has been suggested as a promising treatment. HCQ has been prescribed to millions of people for other diseases with minimal toxicity, but recent reports have demonstrated cardiotoxicity in patients with COVID-19. Therefore, identifying the underlying mechanism(s) of cardiotoxicity could arm physicians with the necessary information to both identify and increase the monitoring of SARS-CoV-2 infected patients experiencing adverse events with these pharmacologic therapies and design interventions to mitigate their negative effects. This project advances the understanding regarding the molecular mechanisms associated with HCQ-induced cardiotoxicity in hiCMs derived from heathy and diseased patients and thus fosters the identification of novel therapeutic targets and development of mechanism-based therapies to attenuate HCQ cardiotoxicity in COVID-19 patients.

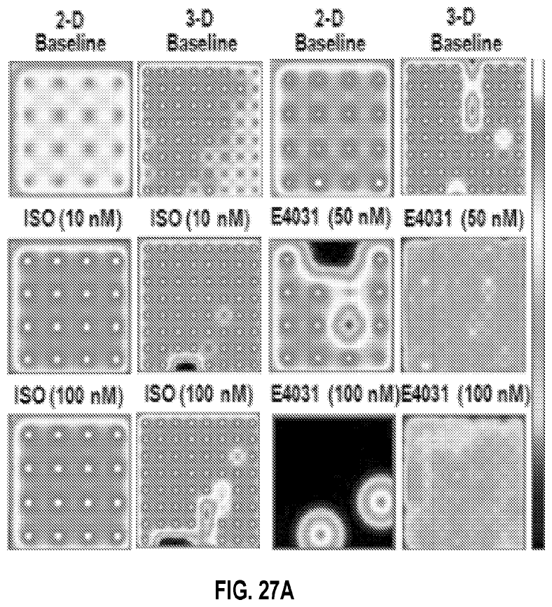

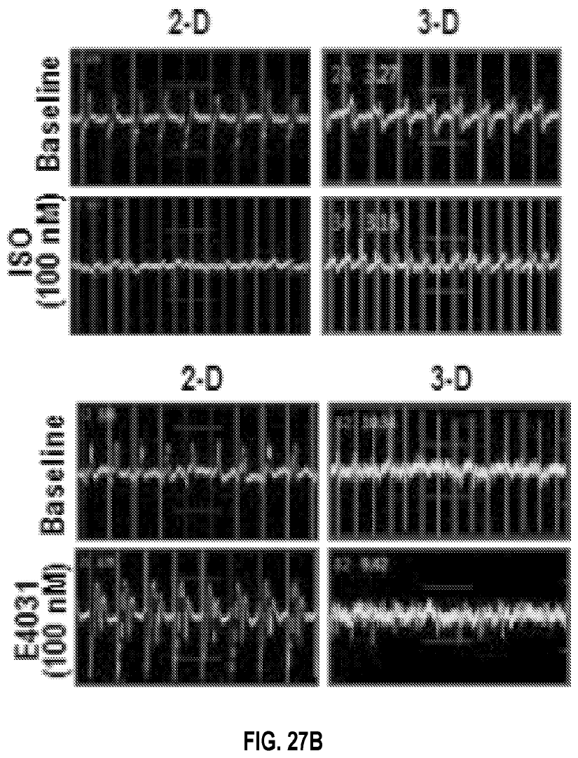

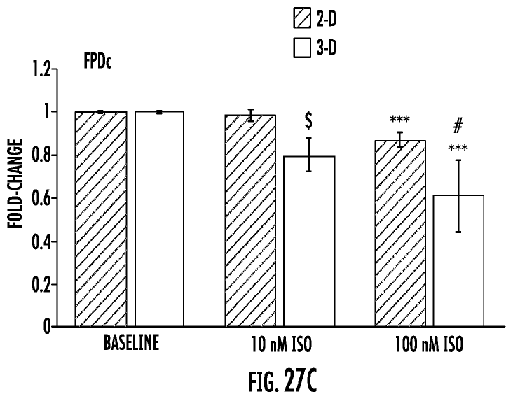

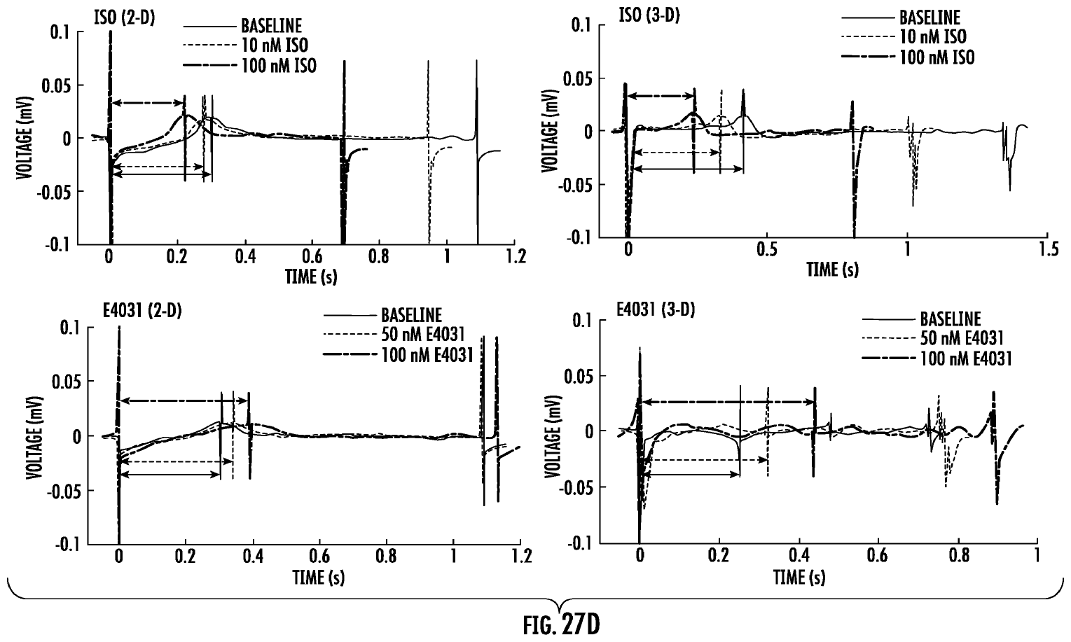

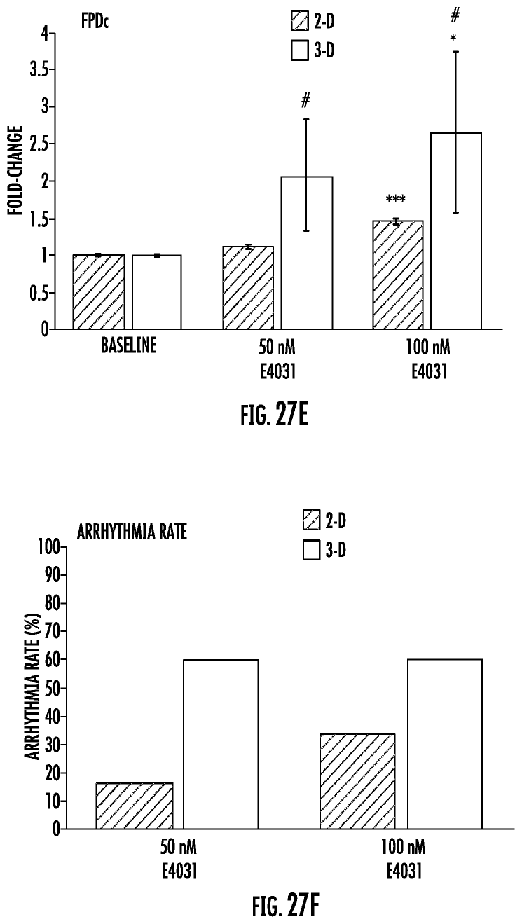

The FDA has recently issued an Emergency Use Authorization (EUA) to use HCQ in patients hospitalized with COVID-19. However, the underlying molecular mechanism of HCQ-induced cardiotoxicity is not fully understood. Therefore, this study is the first to investigate the molecular mechanism of HCQ-induced cardiotoxicity in heathy and diseased human-iPSC cardiomyocytes. The study is innovative for the following reasons: 1) Use of human iPSC-derived cardiomyocytes from both normal and diseased (DCM) patients; 2) transduction with psuedotyped virus particles expressing SARS-CoV-2 proteins instead of live viruses to mimic the disease phenotype, which also eliminates the need for a BSL3 facility; 3) data from in situ experiments with 3-D cardiac scaffolds (FIGS. 26 & 8); 4) Multi electrode array (MEA) platform to assess physiological function in cardiomyocytes (FIG. 27); 5) RNA sequencing and miRNA profiling to assess the molecular mechanism underlying HCQ-induced cardiotoxicity. Overall, the planned approach and cutting-edge tools fill an important gap in knowledge regarding the molecular mechanism of HCQ-induced cardiotoxicity and facilitate the development of knowledge-based therapeutic avenues for COVID-19.

Evaluate the Functional Effect of HCQ on hiCMs Derived from Healthy and Diseased Patients on a 3-D Scaffold In Vitro.

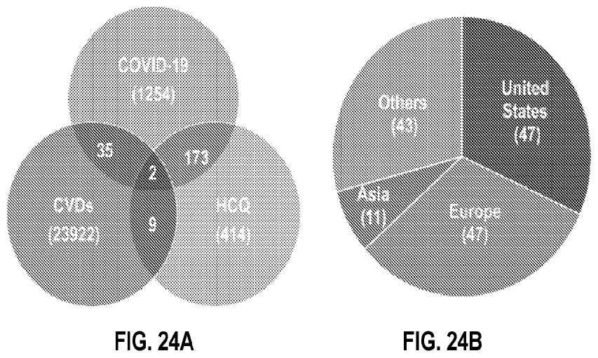

Hydroxychloroquine (HCQ) has been propositioned as a therapeutic drug for SARS-CoV-2 patients. A number of clinical trials are underway to test the safety and efficacy of HCQ in patients (FIG. 24). In fact, a randomized clinical trial showed improved recovery of patients following administration of HCQ for 5 days. However, previous reports indicating the HCQ-induced cardiac dysfunction and the higher mortality in SARS-CoV-2 patients with an underlying cardiovascular disease has raised concerns about the safety of HCQ in patients with underlying cardiovascular disease and an urgent need to investigate molecular mechanisms of HCQ-induced cardiotoxicity in COVID-19 patients. Although uncommon, severe HCQ-induced cardiotoxicity has been reported in patients after prolonged administration of the drug for treatment of rheumatological disorders, connective tissue diseases, and dermatological disorders. HCQ treatment has also been linked to QT prolongation in a cohort of 90 patients with SARS-CoV-2, and ventricular arrhythmias. Additionally, mitochondria can be a source of pro-inflammatory signals but their significance in the inflammatory response is not well understood. Therefore, studies investigating the direct effect of HCQ on cardiomyocyte function and their exact mechanism of action is currently lacking.

HiCMs have been extensively used for disease modeling, toxicity studies, and drug screening. More importantly, they can be an autologous source of cells from patients for conducting ‘clinical trials in a dish’, since they re-capitulate a majority of the drugs' effect observed in vivo. Additionally, 3-D cultured hiCMs have been found to mimic drugs responses in a sensitive manner similar to the native heart tissue. Hence, normal and DCM hiCMs cultured in a 3-D engineered cardiac scaffold can be used as an in situ model to dissect out the effect of HCQ treatment on cardiac function in patients with DCM and underlying cardiovascular disease. The present study assesses if HCQ induces a higher cardiomyocyte dysfunction/cardiotoxicity in DCM hiCMs (vs normal), with or without viral transduction.

The approach involves testing the effect of HCQ treatment on hiCMs from healthy and DCM patients (FIG. 25). HiCMs from normal and DCM patients are cultured (Fujifilm Cellular Dynamics, Cat #R1007 and R115) on 3-D polycaprolactone (PCL)-gelatin co-axial nanofiber scaffolds. hiCMs are seeded at a density of 500,000 cells/cm², this protocol is well established (FIG. 26 & FIG. 8). The functionality of the hiCMs is evaluated after 24 hours of treatment with HCQ (1-10 μM) via MEA analysis as described in FIG. 27. Also, the hiCMs seeded on a 3-D cardiac patch was very responsive to increasing concentrations of Isoproterenol and E4031 (FIG. 27). In addition, mitochondrial function, LDH, caspase, and TUNEL assays are performed to assess oxidative stress and cytotoxicity post-treatment in vitro. Pseudotyped viral particles expressing SARS-CoV-2 S-protein are used for cell transduction (for 12 hours) to mimic the disease phenotype. Untreated hiCMs serves as control.

1.1. Generation of pseudotyped SARS-CoV-2 viral particles: Pseudotyped virus particles expressing the SARS-CoV-2 S-protein are generated using a three-plasmid co-transfection strategy. Highly competent HEK cells are transfected with plasmids encoding (a) MLV core genes (gag, pol), (b) firefly luciferase reporter gene, an MLV MJ-RNA packaging signal, and 5′- and 3′-flanking MLV long terminal repeat (LTR) regions and (c) the SARS-CoV-2 S-protein. The infectivity of the pseudotyped viral particles are assessed by quantification of luciferase activity after transduction, as described previously. A comparative assessment of the susceptibility of normal and DCM hiCMs to viral infection is performed. This provides a comprehensive understanding of the vulnerability of patients with underlying CVD to SARS-CoV-2 infection.

1.2. Evaluate the electrophysiological changes in HCQ treated hiCMs in 3-D cultures: To determine the role of HCQ-mediated cardiomyocyte dysfunction, cardiomyocyte function is evaluated by analyzing its action potential and any changes in its biophysical properties. The electrophysiological changes occurring hiCMs are assessed by measuring field potentials (MEA) and action potentials (patch clamp).

a) Multi electrode array (MEA) system for electrophysiological analysis and HCQ dose optimization studies: MEA systems provide a non-invasive user-friendly platform for detailed electrophysiological analysis of human iPSC cardiomyocytes including drug testing to identify targets and the assessment of proarrhythmic risk. A dose-response curve is established to determine the effect of HCQ treatment on hiCMs. Briefly, normal and DCM hiCMs are treated with different concentrations (1, 2, 5, and 10 μM) of HCQ in a 24-well MEA plate (n=4/group/dose) in to determine the optimum dose to be used for further experiments. The hiCMs cultured in 2-D or on 3-D patches are assessed for spike amplitude, beat period, field potential duration (QT interval), beat irregularity, and conduction velocity using Axion Biosciences Maestro Edge MEA system (FIG. 27). This system and it use of multiwell plates allows for simultaneous group testing over time in a gas- and temperature-controlled environment. Comparison is performed between untransduced and pseudotyped virus-transduced hiCMs from normal or DCM patients after treatment with HCQ for 48 hours. Similar assessment is done for hiCMs cultured on 3-D patches.

b) Patch clamp analysis of action potentials: The precise involvement of ion channels is investigated by electrophysiological analysis. For this, hiCMs are enzymatically dissociated from cultures into single cells and plated at a low density on Matrigel-coated coverslips. Electrophysiological experiments are performed after dissociation of spontaneously beating single cells. Action potentials re recorded at 36±0.5° C. using an Axopatch 200B amplifier (Molecular Devices, Sunnyvale, Calif., USA) and all potentials are corrected for the estimated liquid junction potential. Action potentials are measured by the amphotericin-perforated patch-clamp methodology. Both spontaneous APs and APs elicited at 1 Hz are recorded by overdrive stimulation with 3 ms, 1.2×threshold current pulses through the patch pipette. Cardiomyocytes are perfused with optimized concentrations of HCQ in the bath solution. Assessments are performed to analyze cycle length, maximum diastolic potential (MDP), maximum AP upstroke velocity (Vmax), AP amplitude (APA), AP plateau amplitude (measured 20 ms after initiation of the action potential upstroke), and AP duration at 20%, 50%, and 90% repolarization (APD20, APD₅₀, and APD₉₀, respectively). Parameters from 10 consecutive APs are averaged.

1.3. Assess mitochondrial function in HCQ treated hiCMs in 3-D cultures: HCQ is known to cause mitochondrial dysfunction, resulting in an alteration in ATP production and apoptosis. The role of HCQ in mitochondrial dysfunction is elucidated. Normal and DCM hiCMs are used for measuring mitochondrial ROS, calcium waves, and ATP production. Here, the study tests whether HCQ causes depolarization of ψₘᵢₜₒ and thus decreases electromotive forces for mitochondrial Ca²⁺ uptake and modulates mitochondrial function. To test this potential mechanism, mPTP opening, ROS, ψₘᵢₜₒ, mitoCa²⁺ concentrations, mitochondrial, and cristae morphology in human iPSC-CMs are measured. Briefly, mPTP formation is analyzed by using calcein cobalt protocol by incubating hiCMs from all experimental groups in calcein-acetoxymethyl ester (calcein-AM) and CoCl₂ which results in calcein fluorescence in mitochondria. For the analysis of ψₘᵢₜₒ, cells are loaded with 20 nM tetramethylrhodamine methyl ester (TMRM), a cell-permeant fluorescent dye that collects in active mitochondria with intact membrane potentials. To monitor mitochondrial Ca²⁺, hiCMs are loaded with 5 μM rhod-2 AM, which increases in fluorescent intensity upon binding Ca²⁺. To further corroborate rhod-2 AM signals, mitochondria-targeted Ca²⁺-sensitive biosensor Mitycam, or GCaMP, are also used. The current study also measures ATP by using a luciferase assay to determine the effect of HCQ on ATP production quantified by luminescence detection.

1.4. Evaluate HCQ-induced cytotoxicity in hiCMs on 3-D patches: HCQ-included cytotoxity is evaluated in both normal and DCM hiCMs under unstimulated and stimulated conditions. HCQ-induced cytotoxicity can be determined by indirectly estimating apoptosis-associated markers like caspase or LDH. For this, hiCMs cultured on 3-D patches are assessed for cell viability using the Promega MultiTox-Fluor Multiplex Assay per the manufacturer's instructions following treatment with HCQ. Furthermore, HCQ-induced apoptosis is measured using XTT and TUNEL assay. Additionally, the caspase activity in the hiCMs before and after drug treatment is assessed using the Caspase-Glo® 3/7 Assay (Promega). Further, the LDH release assay is performed with the different treatment groups' conditioned media as a measure of the apoptosis induced in cells.

The binding of the pseudotyped virus particles (expressing S-protein) to the ACE2 protein expressed on hiCMs can result in the recapitulation of SARS-CoV-2-induced changes in cardiomyocyte function. Level of luciferase activity in DCM hiCMs can be higher compared to normal hiCMs, as higher ACE2 expression has been reported. Following treatment of hiCMs with HCQ, considerable changes in the electrophysiological function (assessed by MEA and patch clamp), increased cell death, and decreased cell viability can be observed. Significant changes in the mitochondrial function caused by alterations in ψₘᵢₜₒ can be observed. Since this is the first study to assess the involvement of ion channels in HCQ-induced modulation of cardiomyocyte function, the particular ion channel(s) affected is ascertained.

Identify Molecular Mechanisms Responsible for HCQ-Induced Cardiotoxicity in Normal and Diseased hiCMs In Vitro.

The molecular mechanism(s) underlying HCQ-associated cardiotoxicity are unknown and whether mechanism(s) differ in healthy versus diseased patients has not been investigated. Therefore, a molecular analysis of HCQ-induced changes in normal and diseased cardiomyocytes is needed. SARS-CoV-2 virus attaches and enters cells via ACE2. Cardiomyocytes express high levels of ACE2, which is internalized upon SARS-CoV-2 entry thus reducing the amount of functional ACE2. Therefore, transduction of cells with a pseudotyped SARS-CoV-2 virus mimics this change and the studies shown herein determines whether and how that affects the molecular changes induced by HCQ. These results can be used to identify additional parameters in COVID-19 patients that need be monitored during treatment.

hiCMs cultured in a 3-D microenvironment can be used as an in situ model to identify molecular mechanism(s) underlying HCQ treatment in human cardiomyocytes. The comparison of normal and DCM hiCMs can unravel novel differential molecular changes responsible for increased cardiotoxicity seen in HCQ treated patients with cardiovascular disease. HCQ treatment can result in differential regulation of cardiotoxicity-associated signalling molecules in normal and DCM hiCMs.

The molecular basis of HCQ-induced cardiotoxicity is investigated using hiCMs derived from healthy patients and diseased (DCM) patients. hiCMs are treated with HCQ with/without transduction with SARS-CoV-2 pseudovirions. This study analyzes cardiac gene expression, ACE2 expression, ultrastructural morphology, paracrine secretion, miRNA profiling, and paired-end RNA sequencing. Normal and DCM hiCMs are cultured onto 2-D slides for TEM and on 3-D scaffolds for all other analyses. hiCMs are treated with/without SARS-CoV-2 pseudovirions for 12 hours then treated with HCQ for 48 hours. hiCMs are analyzed for cardiac genes and paracrine genes with qPCR arrays. Transmission electron microscopy (TEM) is used to investigate any ultrastructural changes in hiCMs. Conditioned media is analyzed for cytokine secretion by ELISA. hiCMs also undergo miRNA profiling and paired end total RNA sequencing to identify pathways affected by the drug treatments. Cardiac gene qPCR and TEM inform whether cardiomyocyte-specific changes occur and the molecular and/or structural level. Paracrine expression and secretion determine intercellular communication changes occurring during treatment. miRNA profiling and RNA sequencing data are cross analyzed to determine any interacting networks modulated by HCQ treatment. Untransduced and untreated hiCMs can serve as experimental controls.

2.1. Analysis of expression of cardiac genes and ACE2 in normal and DCM hiCMs in response to HCQ: A cardiac gene expression analyzed in normal and DCM hiCMs (untreated/vehicle control, HCQ; with/without pseudovirus transduction). A custom cardiac gene qPCR array (Qiagen, MD) is designed to easily analyze 26 cardiac-related genes to initially investigate changes in gene expression. Included in the array are cardiac contractile genes, ion (K⁺, Na⁺, Ca²⁺) channel genes, mitochondrial genes, cell adhesion genes, and transcription factors. Data are normalized to the geometric mean of 3 housekeeping genes and expression is calculated relative to normal untreated hiCMs using the 2^{-ΔΔCt} method. These results determine the molecular effect of HCQ treatment on the cardiomyocytes and provide a foundation for mechanistic studies. The expedient nature of this array enables the surveillance of changes in gene expression, which can be used to adjust the dose or duration of treatment used. Additionally, the expression of ACE2 is studied with immunofluorescent staining (ab87436, Abcam, MA) in untransduced and pseudotyped virus transduced normal and DCM hiCMs, with/without HCQ treatment.

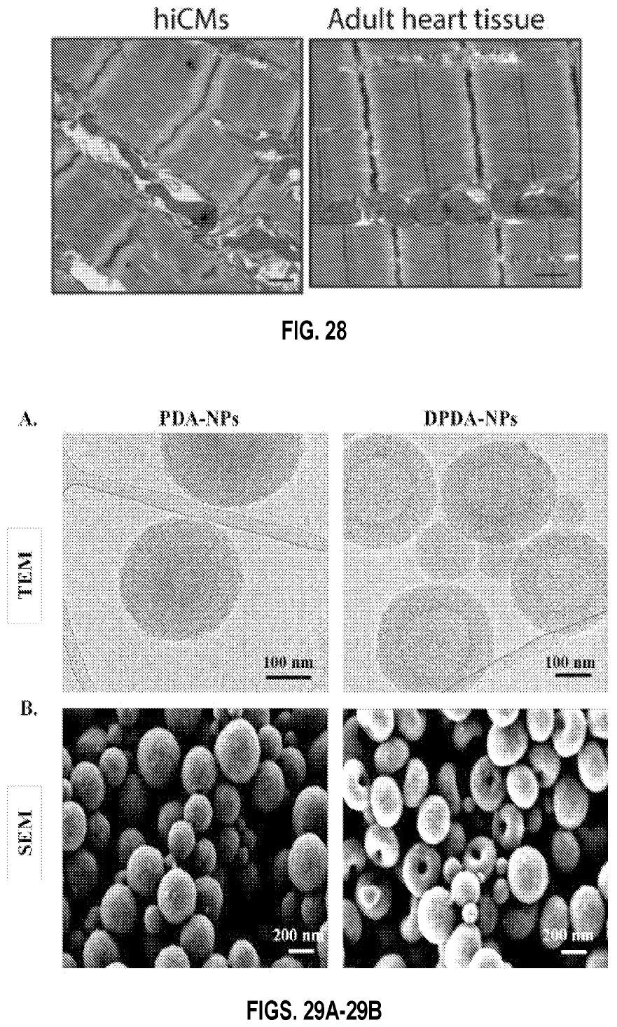

2.2. Transmission electron microscopy (TEM) for ultrastructural analysis of normal and DCM hiCMs treated with HCQ: Changes in cellular function usually correlate with changes in structure. TEM imaging of hiCMs treated with HCQ with/without pseudoviral transduction determines if changes in cardiomyocytes occur at an ultrastructural level. Study demonstrated similar ultrastructure between healthy hiCMs and non-failing adult human heart tissue (FIG. 28), strengthening the biomimetic properties of this cell model. hiCMs are seeded onto Permanox-coated chamber slides for TEM imaging, transduced with/without pseudotyped virus and incubated for 12 hours, then subjected to HCQ treatment for 48 hours. hiCMs are fixed with 2.5% glutaraldehyde with sucrose in phosphate buffer and processed for TEM.

2.3. ELISA analysis of cytokine/chemokine and cTnI secretion in normal and diseased hiCMs in response to HCQ: As severely ill COVID-19 patients have increased plasma levels of proinflammatory cytokines, analysis of HCQ-induced cytokine secretion by hiCMs elucidates whether the drug treatment exacerbates the inflammatory state. Moreover, comparing secretion of normal and diseased hiCMs can determine if the paracrine response to HCQ is similar in these two populations. Normal and DCM hiCMs are transduced with/without pseudotyped SARS-CoV-2 virus, incubated for 12 hours, and then treated with/without HCQ for 48 hours. Conditioned media collected and spun down to remove cellular debris. It is analyzed at the protein level for 32 growth factors, cytokines, and chemokines with an ELISA array (Signosis, EA-4002). This analysis gives a clear depiction of functional signaling from each group and includes IL-6, IL-10, IL-2, IFNγ, IP-10, as MIP-1α, expression of which correlate with severe COVID-19 disease. Furthermore, cardiac troponin-I is detected in plasma as a result of cardiac injury and high serum levels have been reported in severely ill COVID-19 patients. Therefore, conditioned media from the same groups are tested for human cardiac troponin-I using an ELISA kit, (Abcam, ab 200016), detection of which is indicative of cardiomyocyte damage.

2.4. miRNA profiling of normal and diseased hiCMs in response to HCQ: MicroRNAs are well known regulators of gene expression and profiling their expression can elucidate pathways and genes that are affected by a given treatment. NanoString's nCounter Human miRNA v3 panel were previously utilized to profile miRNA expression changes during maturation of hiCMs over time in culture. This panel analyzes expression over 800 human miRNAs and requires a low amount of input RNA. Differential miRNA expression are analyzed using NanoStringDiff and the count data is characterized by a generalized linear model of the negative binomial family and allows for multi-factor experimental design. Pathway analysis of miRNA targets is performed by miRanda and Ingenuity Pathway Analysis (Qiagen).

2.5. RNA sequencing in normal and diseased hiCMs in response to HCQ: RNA sequencing provides an unbiased approach to possible mechanistic candidate genes modulated by HCQ treatment in heathy and diseased cardiomyocytes. Paired-end sequencing is performed on the total RNA obtained from normal and DCM hiCMs in each treatment group (untreated/vehicle control, HCQ; with/without pseudotyped virus). The reads are trimmed following a quality control analysis using FATQC. The trimmed reads are then disambiguated and mapped against the human reference genome and rat reference genome from NCBI using BBMap tool. This helps isolate reads that uniquely map to each species from the mixed species RNAseq data. The reads that uniquely map to each species' genomes are then re-aligned to their respective genomes using the splice aware RNASeq aligner HISAT2 (v2.2.0) with default settings to generate binary alignment map (BAM) files. The BAM files are used to determine gene-level and exon-level counts using the SummarizeOverlaps function from the GenomicAlignments package in R for each species. The counts are used to determine differentially expressed genes using DESeq2. Pathways enriched among the significantly differentially expressed genes (adjusted P-value≤0.05) are identified using Ingenuity Pathway Analysis (Qiagen). In parallel, Gene Set Enrichment Analysis (GSEA) are used, performed using SeqGSEA, to include differential splicing and differential gene expression in pathways analysis in the rat hearts following the different treatments. Differentially expressed miRNAs are correlated to the expression of their target mRNAs using a miRnet to generate a miRNA-mRNA network. This analysis provides a direction towards a more viable list of candidate genes affected by HCQ and pseudotyped virus transduction with an associated underlying miRNA-regulated mechanism.

Novels genes can be identified mediated by HCQ treatment and involved in cardiotoxicity. Increased expression of ACE2 is observed in DCM hiCMs. As the 3-D cardiac patches mimic the native environment, increased secretion of inflammatory cytokines with pseudotyped viral transduction is observed. This information provides the understanding of HCQ treatment in patients, with/without COVID-19, and with underlying CVD.

### Example 6. Photothermal Welding of Pro-Angiogenic Cardiac Patch for Myocardial Repair

The myocardial infarction causes the formation of an akinetic scar on the heart leading to impaired contractility and conductance. This often leads to cardiac remodeling causing insufficiency, the pharmacological approaches that are available for preventing cardiac remodeling are limited and often come with long-term adverse effects. Thus, there is an urgent need to develop therapeutic modalities that can aid in repairing the scarred cardiac tissues and enhance the localized conductance. In the current example, the applicability of polydopamine nanoparticles (PDA-NPs) as a cardiotonic agent was demonstrated. The preliminary in vitro testing performed on HiPSC-CM's showed enhanced conductance, suggesting its use in akinetic scar region for improving contractility. Moreover, the proof-of-concept was demonstrated for sustained delivery of bioactive proteins (bFGF) that can be used for cardiac repair. Furthermore, the suture-free binding of PDA coated cardiac patch (Coaxial PCL-Gelatin) was demonstrated by employing a near-infrared (NIR) light laser. Taken together these findings suggests PDA as a versatile biomaterial that can be used for cardiac repair.

1. Background

Acute myocardial ischemia (MI) is caused by the blockage of coronary arteries supplying to the ventricles. Following an MI, a large percentage of cardiomyocytes are lost leading to a catastrophic downstream cascade. Primarily, the inflammatory response following an event of MI leads to the accumulation of cytotoxic ROS, which further exacerbates the damage to the heart tissue. In addition to it, the enhanced activity of matrix-degrading enzymes (MMP's) following an MI, is responsible for ventricular wall thinning and remodeling. Taken together, all these events lead to the formation of akinetic scar hampering cardiac contractility (end-stage disease). Current pharmacological interventions are limited in preventing cardiac remodeling, thus a radical therapeutic strategy is warranted for cardiac repair and ameliorating the damage.

One of the most promising yet challenging therapeutic interventions is the implantation of stem cell-derived cardiomyocytes. Initial preclinical studies have demonstrated functional improvements in the damaged myocardium post-implantation; however, the survival and integration of implanted cells to the host myocardium present a major challenge. Despite the poor engraftment, improvements in cardiac function were attributed to the paracrine function of these cells. To prevent cell death post-implantation, electrospun nanofibers have been used as a scaffold for CM's implantation. With the advent of electrospinning combined with smart biomaterials, researchers were able to develop beating cardiac patches that mimic the activity of available myocardium. However, the implantation of such scaffolds requires invasive surgical procedures and suturing on the myocardium which is a deciding factor for considering implantation of the patch, given the risks and benefits.

In the current example, a smart cardiac patch was developed, capable of suture-free engraftment by using NIR light loaded with proangiogenic factors and anti-inflammatory agents for cardiac repair. With the advent of minimally invasive robotic surgeries, suture-free engraftment of cardiac patches can significantly expand the therapeutic spectrum of cardiac patches not only limiting them for end-stage cardiac patients.

2. Materials and Methods

2.1 Materials

Dopamine, Trimethyl benzene (Mesitylene), pluronic F-127, tris base were procured from SigmaAldrich. All the other reagents used were of analytical grade.

2.2 Methods

Preparation of PDA-NPs and DPDA-NPs

PDA-NPs were prepared by the oxidative polymerization of dopamine monomer, briefly, 60 mg of dopamine was weighed and dissolved in a mixture of water/ethanol (65 mL/60 mL) and was stirred. To this mixture, 10 mL water containing 90 mg of tris base was introduced and the solution was stirred overnight. The change in color of the solution from light brown to black confirmed the formation of nanoparticles. The following day, nanoparticles were pelleted by centrifugation at 15000 rpm for 10 min and lyophilized.

The mesoporous PDA-NPs (DPDA-NPs) were prepared by following published literature, Mesitylene was used as a pore-forming agent, 420 μL of and 320 mg of pluronic F-127 was added to the water/ethanol mixture and stirred. The resultant emulsion was mixed for 30 min and following which dopamine (60 mg) was added and polymerized overnight by following a similar procedure. The formed DPDA-NPs were washed three times in a solvent mixture of ethanol/acetone (2:1) to remove Mesitylene and sonicated for 10 min (2 sec on/off cycles).

Preparation of bFGF Loaded PDA-NPs and DPDA-NPs

The PDA-NPs and DPDA-NPs were incubated with bFGF and stirred overnight under 4° C., the ratio of nanoparticle and bFGF was 1:100. The protein loading was performed by suspending nanoparticles in 10 mM Tris buffer (pH 8). The following day, 0.1% BSA was added to the same mixture and stirred to disperse the aggregates. The prepared nanoparticles were subjected to centrifugation at 15000 rpm for 10 min (thrice) and the supernatant was collected, later, the nanoparticles were suspended in PBS and stored at −20° C. The unentrapped bFGF was estimated from the collected supernatant solutions by using an ELISA kit, and loading efficiency was calculated.

PDA-NPs and DPDA-NPs Characterization

TEM and SEM Imaging

The nanoparticles were subjected to Cryo-TEM, the samples were suspended in distilled water and cast for imaging. Similarly for SEM imaging, the samples were drop-casted on conductive carbon tape and sputter-coated for imaging.

UV-Vis Spectroscopy, NTA and Zeta Potential Analysis

The prepared nanoparticles were subjected to UV-Vis spectroscopic analysis the samples were diluted to a concentration of 100 μg/mL and spectra were recorded from 400-900 nm. For NTA, the nanoparticle solution was diluted to a ratio of 1:1000, for evaluating the size distribution and nanoparticle concentration. Lastly, the nanoparticle samples were subjected to zeta-potential analysis to evaluate the surface charge.

bFGF Release Kinetics

The bFGF release from nanoparticles was conducted by direct suspension method, the nanoparticles (1 mg) were suspended in PBS. The sample tubes were incubated in a shaking incubator at 37° C. (100 rpm). The samples were drawn at stipulated time points by subjecting the sample tube to centrifugation at 15000 rpm for 10 min and fresh PBS was added to the pellets. The collected supernatant solution was subjected to ELISA analysis to estimate the protein concentration by comparing it with the known standard curve of bFGF.

DPPH Assay

The antioxidant potential of PDA-NPs and DPDA-NPs was evaluated by DPPH assay. A stock solution of DPPH in ethanol was prepared (100 μM), 100 μL from the stock solution was added to the 96-well plates. Each well was further added with 20 μL of water-containing nanoparticles in varying concentrations (1, 5, 10, 25, 50 μg/ml) respectively. The decrease in absorbance following the addition of test samples was recorded and the percent DPPH quench was plotted. Ascorbic acid with the same concertation range was used as control.

Biocompatibility

The biocompatibility of prepared nanoparticles was evaluated on HUVEC cell line by XTT assay. Briefly, the cells were plated in 96-well plates with a seeding density of 8000 cells/well (DMEM+10% FBS). Following day fresh medium containing PDA-NPs/DPDA-NPs (1, 5, 10, 25, 50 μg/mL) was added and allowed to incubate for 48 h. Later, the cells were washed with PBS and XTT reagents containing media were added. Cells were further incubated for 2 h and absorbance was recorded and % viability was calculated.

Intracellular Uptake

Immunofluorescence imaging was performed on hiPSC-CMs cultured on coverslips. The hiPSC-CMs were incubated with PDA-NPs tagged with calcein for 4 hrs after which they were washed twice with DPBS and processed for immunostaining as described previously. Briefly, the cells were fixed in 4% PFA for 15 min at RT, permeabilized using 0.2% Triton X-100 and incubated in a blocking buffer containing 1% bovine serum albumin (BSA, Sigma-Aldrich, MO). The cells were incubated with the rabbit anti-Troponin T (1:200, Sigma-Aldrich, MO, Cat #HPA017888) overnight at 4° C., followed by the anti-rabbit Alexa Fluor® 594 (1:1000, Cell Signaling Technology, MA, Cat #8889S) for 1 h at RT in the dark. The cells were counterstained with DAPI (Thermo Fisher Scientific, MA) and the coverslips were mounted over glass slides using ProLong™ Gold Antifade Mountant (Life Technologies, MA). The cells were then imaged on the Olympus FV3000 (Olympus Life Sciences, PA) confocal microscope.

Calcium Transient Imaging

For calcium imaging, 40000 hiPSC-CMs were plated on glass coverslips. Calcium transients in the hiPSC-CMs were visualized using the Fluo-4-AM (Thermo Fisher Scientific) as per the manufacturer's instructions. Briefly, 0.5 mL DMEM containing 10 uM of Fluo-4-AM dye, was added to each 35-mm dish, and the cells were incubated for 1 h at 37° C. in the dark. The medium was then replaced with fresh DMEM and the cells were further incubated for 30 min before imaging on a Leica fluorescence microscope. Calcium transients were recorded before and after the addition of 25 ug PDA-NPs. All captured videos were then analyzed using the ImageJ software to determine the fluorescence intensity changes in the cells

Multi Electrode Array (MEA) Analysis

The field potentials of hiPSC-CMs treated with different concentrations of PDA-NPs were measured using an MEA system. For this, hiPSC-CMs were either cultured directly on 24-well MEA plates (M384-tMEA-24W, Axion Biosystems, Atlanta, Ga., United States) having 16 PEDOT microelectrodes per well (as described previously; Kumar et al., 2019) for 2 weeks. The plate was equilibrated in the MEA system (Maestro Edge, Axion Biosystems, Atlanta, Ga., United States) for 30 min in 5% CO2 with a humidified atmosphere at 37° C. The baseline was recorded for each well for 5 min. After which the hiPSC-CMs were treated with different doses of PDA-NPs (5, 10, 25 μg/mL) The plates were equilibrated for 5 min after the addition of NPs and the field potentials were recorded for 5 min. AxIS Navigator Version™ 1.4.1.9 was used for data recording while the Cardiac analysis tool version 2.1.10 (Axion Biosystems, Atlanta, Ga. United States) was used for data analysis. The beat period, field potential duration (FPD) and conduction velocity were calculated. Further, Fredericia's correction was applied to the FPD, to interpret the effect of drugs on the QT interval. Data are expressed as mean SD (n=3).

In Ovo CAM Assay

The angiogenic potential of bFGF loaded PDA-NPs and DPDA-NPs was evaluated by using fertilized chicken eggs. The eggs were incubated in a hatching incubator for 5 days, following the incubation the viable eggs were selected and grouped as follows (bFGF+Geltrex, bFGF-PDA-NPs+Geltrex, bFGF-DPDA-NPs+Geltrex and only Geltrex). A window was made on the eggshell and a sterilized nylon mesh was placed inside the egg. The test samples were loaded on the nylon mesh and the window was covered by placing a 35 mm plate and secured by using tape. The eggs were further incubated for 5 days, following which it was carefully dissected to retrieve the nylon mesh and imaged using a stereomicroscope (Leica).

PDA-NPs Coating on PCL-Gelatin Nanofiber Patches

The electrospun nanofiber patches were crosslinked using 5 mM EDC solution overnight, the following day crosslinked patches were gently washed with 70% ethanol to remove remnant EDC. Later the patches were coated with PDA by two different methods, which is as follows.

For in situ PDA coating, the crosslinked patches were immersed in dopamine solution (2 mg/mL) in water. The pH 8 was adjusted by using 1 N NaOH, and the reaction was conducted for 12 h. Later, the patches were washed with 70% ethanol and PBS. In other instances, the nanofiber patches were coated with PDA-NPs, wherein the crosslinked patches were incubated in tris buffer pH 8 and to it, preformed PDA-NPs (2 mg/mL) were added. The patches were placed on a rocker for 12 h and the following day, patches were washed with 70% ethanol and PBS.

Photothermal Suturing of PDA Coated Patches

NIR-light mediated photothermal suturing of PDA coated patches was performed on isolated mice hearts. The mice's heart was grafted by following the Langendroff technique on an isolated heart perfusion setup. A 30% albumin solution was used as a binding solution, 20 μL of the albumin solution was added on the patch which was then placed on the heart. The PDA-coated patch was then irradiated with 808 nm laser (650 mW) for 20 sec over the borders of the patch. Following the irradiation, the patches were firmly secured on the heart muscles.

3. Results and Discussion

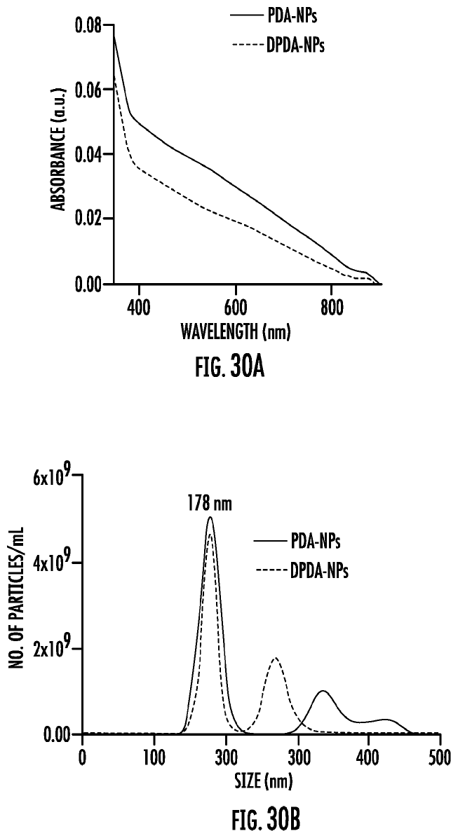

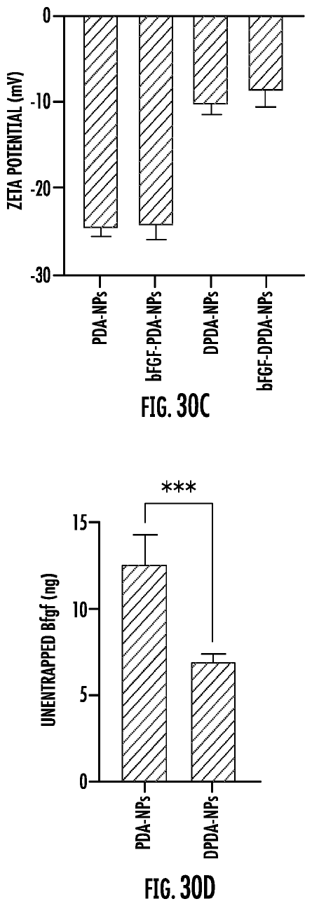

Polydopamine nanoparticles have been extensively researched as a photothermal transducer for photothermal therapy of localized tumors. It has also demonstrated adhesive properties and can bind to a variety of surfaces. In the current example, the applicability of was investigated as, a protein carrier for angiogenesis, a photothermal transducer for localized photothermal suturing, moreover, the effect of polydopamine treatment oncardiomyocytes was also evaluated. The PDA-NPs were prepared by oxidative polymerization of dopamine monomer that formed spherical nanoparticles as shown in FIGS. 29A and 29B. While The DPDA-NPs exhibited mesoporous structure mimicking a donut. This could be due to template inclusion during the polymerization, which was then extracted to induce pores. The porous nanoparticles are known to exhibits a larger surface area which can be used to graft proteins and small molecules. Unlike mesoporous silica nanoparticles that are non-degradable and exert subacute toxicity, mesoporous PDA could be used as a potential alternate. Both PDA-NPs and DPDA-NPs exhibited a wide range of absorbance (FIG. 30A) spanning from 400 nm to 800 nm. However, DPDA-NPs showed a decline in absorbance, which could be due to lower yield than PDA-NPs. Later, the NPs were subjected to NTA, it was observed that both PDA-NPs and DPDA-NPs were about ˜170 nm in size, with a slight increase in the particle concentration of PDA-NPs than DPDA-NPs (FIG. 30B). In addition to it, nanoparticle within the range of 200-400 nm was also present. The lower particle density in DPDA-NPs could be due to lower yield, corroborating with UV-Vis findings. Thus, for protein loading, the concentration of PDA-NPs and DPDA-NPs were normalized with particle numbers and proceeded for further experimentation. PDA-NPs are well known to interact with a wide variety of substrates, predominantly by π-π interaction and exhibit a pH-responsive drug release. In the current study, bFGF was interacted with PDA-NPs and DPDA-NPs by electrostatic and π-π interaction. The entrapment efficiency of bFGF in both the nanoparticles was estimated by ELISA (i.e. ˜99%). However, as shown in FIG. 30D, DPDA-NPs showed higher entrapment. Later, the bFGF release was conducted in PBS at 37° C., it was observed that DPDA-NPs exhibited a sustained release when compared to PDA-NPs, which lasted beyond 30 days. This data suggests that DPDA-NPs could be employed for the sustained delivery of bFGF.

Due to the presence of catechol rings, PDA-NPs exert a significant antioxidant potential, it has been reported that PDA-NPs can be effectively employed as a free radical scavenger in disease conditions. The antioxidant potential of PDA-NPs and DPDA-NPs was estimated by DPPH assay. As shown in FIG. 31, the % DPPH quenching was marginally higher in DPDA-NPs when compared with PDA-NPs at 5 min time point, however, by the end of 60 min, the efficacy was similar at 25 μg/mL dose in all the groups, suggesting the antioxidant potential comparable to ascorbic acid. Taken together the data suggests that PDA-NPs and DPDA-NPs can be employed as a delivery agent for angiogenic factors (bFGF) and a potent antioxidant for preventing inflammation.

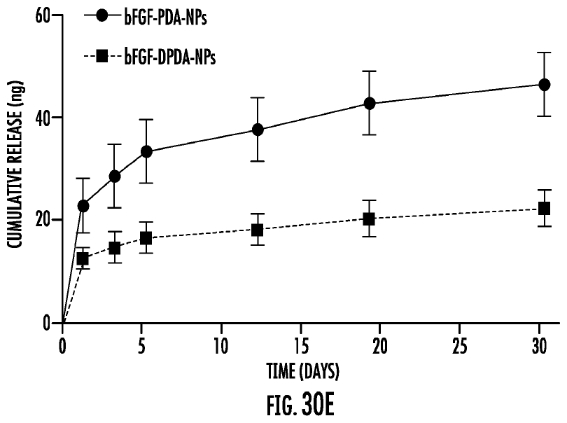

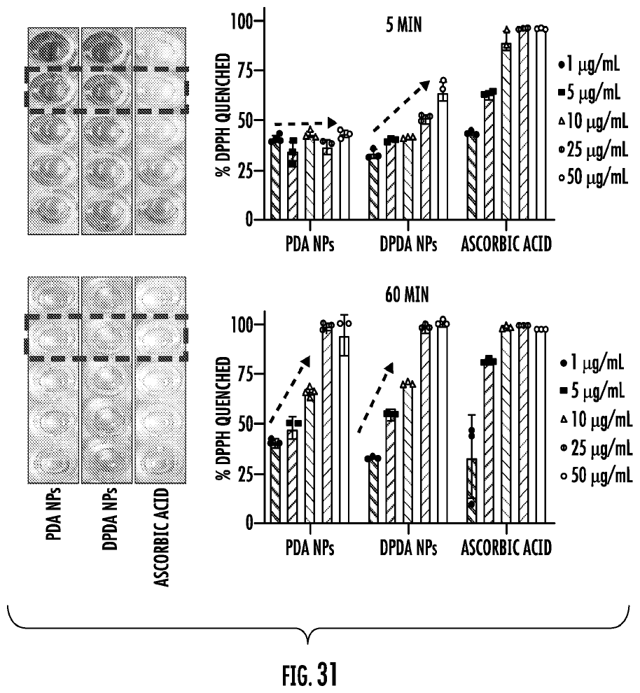

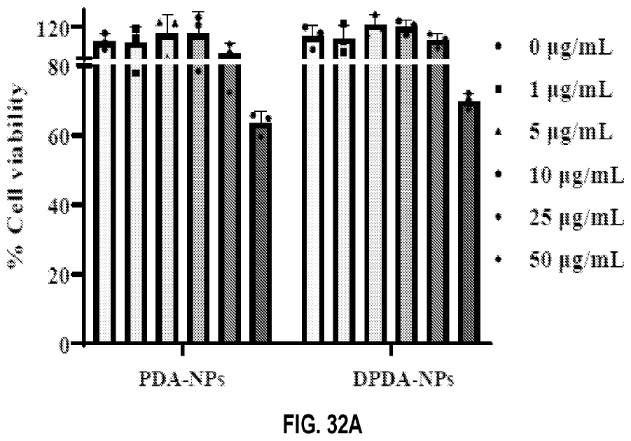

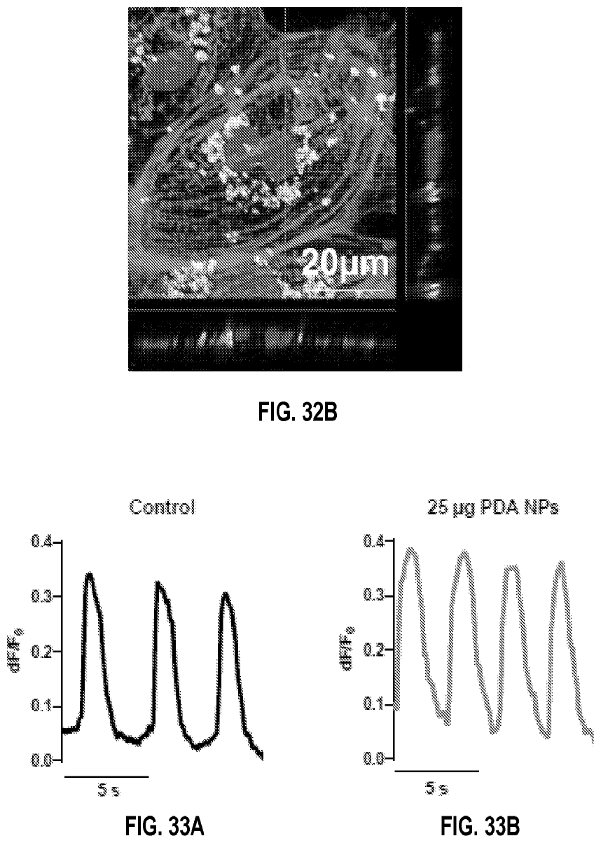

The biocompatibility of PDA-NPs/DPDA-NPs was further analyzed in the HUVEC cell line, both the nanoparticles were biocompatible up to 25 μg/mL (FIG. 32A), however, 50 μg/mL exhibited a decline in the cell viability and thus 25 μg/mL opted as an optimum dose. The effect of PDA-NPs on cardiomyocytes and their intracellular uptake was further investigated. The PDA-NPs were tagged with calcein and subjected to cells. As shown in FIG. 32B, the PDA-NPs were taken up by the cardiac cells surrounding the nucleus without affecting the expression of cardiac troponin T marker. The results demonstrate that PDA-NPs can be employed to deliver cargo to the cardiomyocytes.

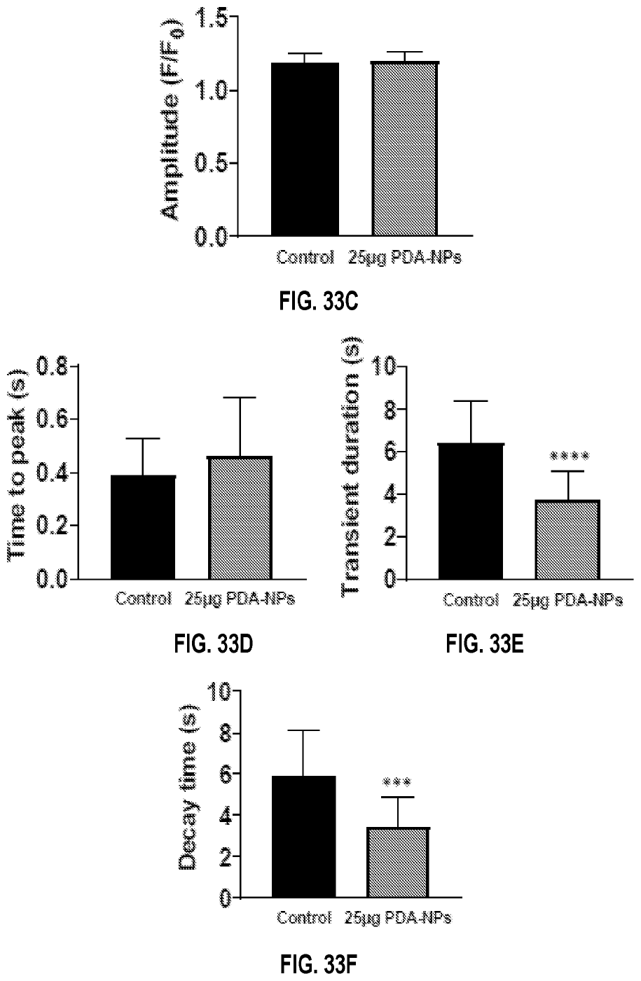

The effect of PDA-NPs on cardiomyocytes was investigated by calcium transient imaging. Interestingly, an increase in calcium transients in the hiPSC-CMs after PDA NP treatment along with a significant decrease in transient duration and decay while no significant changes were seen in the amplitude and time to peak was observed (FIG. 33A-33F). This is indicative of faster repolarization of CMs by PDA NPs. This was also confirmed by MEA analysis where a significant decrease in the beat period was observed with an increase in the dose (FIG. 33G). The inventors are the first to show the drug-like activity of PDA-NPs in CM's.

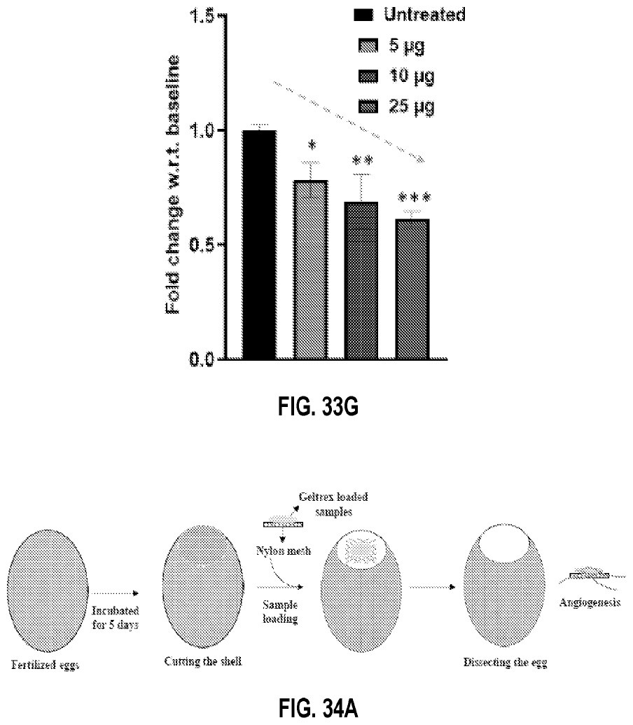

The pro-angiogenic effect of bFGF loaded nanoparticles was then investigated by using in-ovo CAM assay technique. FIG. 34A shows the schematic representing the experimental protocol, Geltrex was used as a carrier material that was devoid of angiogenic factors. As shown in FIG. 34B, the nylon mesh retrieved from the treated embryos shows remarkable angiogenesis in all the treated groups except Geltrex control. Both PDA-NPs and DPDA-NPs entrapping bFGF showed newer blood vessel formation comparable to only bFGF group. The data demonstrate that administration of bFGF loaded PDA-NPs and DPDA-NPs can induce neoangiogenesis.

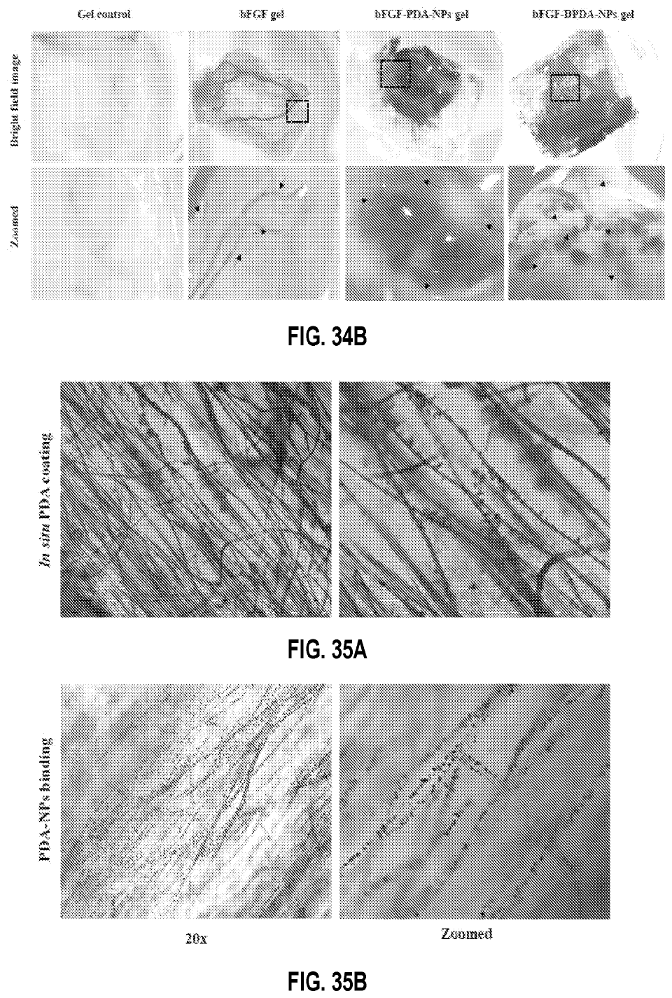

The electrospun nanofiber patches have widely been used as a scaffold for cells and stem cell implants. However, the major limitation with stem cell-based scaffold implants is poor cell survival due to a lack of angiogenesis. Thus, an angiogenic scaffold can be used for both cell and cell-free implants. As demonstrated earlier, the PDA-NPs entrapping bFGF can significantly induce rapid angiogenesis, In the current example, the PDA-NPs were combined and camouflaged with electrospun nanofibers that can be used for inducing angiogenesis. FIGS. 35A and 35B shows the nanofibers coated with PDA polymer and preformed nanoparticles. More interestingly, the PDA-NPs coated patch (FIG. 35B) can be used for bFGF release and localized antioxidant effect. Acute myocardial ischemia can significantly induce intracellular and extracellular oxidative stress, which in turn causes inflammatory response leading to LV remodeling. The presence of PDA-NPs on the nanofiber scaffold can significantly attenuate the ROS and inhibit the inflammation that can aid in preventing cardiac remodeling, thus nanoparticle coated cardiac patches can be a modality in mending damaged hearts.

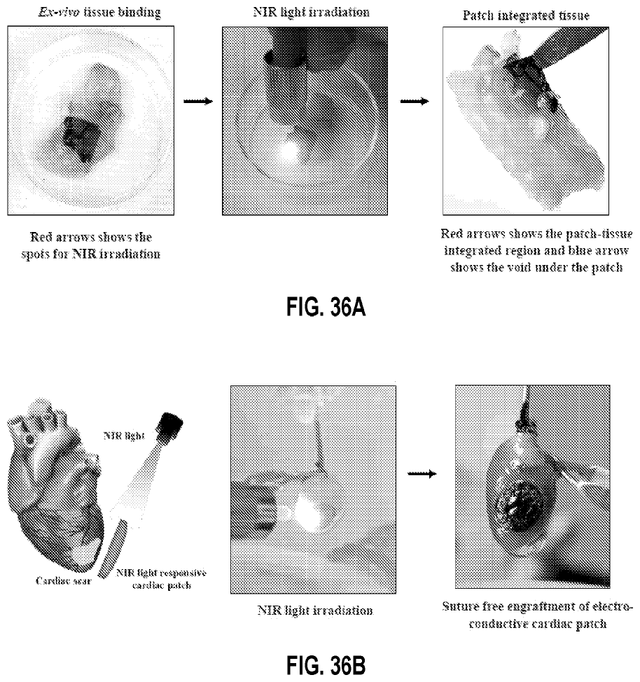

Lastly, one of the most important properties of PDA-NPs is photothermal transduction, due to which the light is converted to heat. In the current example, the localized heat generated was exploited to induce albumin intercross-linking between ECM proteins and PDA-coated patches to form a stable bond. FIG. 36A shows a PDA-coated patch placed on a tissue sample, the NIR light was irradiated at the peripheries of the patch for a short period which leads to the binding of the patch. The patch and tissue integration were localized at spots where NIR light was irradiated. This data demonstrates a minimally invasive approach for nanofiber patch grafting. Similarly, photothermal suturing of PDA coated nanofiber patches on a beating heart was also attempted. As shown in FIG. 36B, brief irradiation of NIR light on the peripheries of cardiac patch resulted in stable integration of patch to the heart tissue, demonstrating the feasibility of PDA coated patch integration on a variety of tissue substrates.

In conclusion, PDA-NPs are a versatile protein delivery agent for the sustained release of angiogenic factor (bFGF) that can be employed for tissue repair and to promote angiogenesis. The antioxidant potential of PDA-NPs and modulation of cardiac contractility when exposed to CMs PDA-NPs based patches were also demonstrated for suture-free tissue integration.

### Example 7. Collagen and Collagen-Based Electrospun Scaffolds for Cardiac Engineering

Electrospun collagen scaffolds have become a common material for many tissue engineering applications Collagen can be extracted from a number of different tissues and a wide variety of organisms including mammals, amphibians, fish, and bird. Additionally, recombinant technologies to produce collagen, specifically, type I, have been developed. Genetic modification of tobacco plants with two genes encoding recombinant heterotrimeric collagen type I, and the human prolyl-4-hydroxylase (P4H) and lysyl hydroxylase 3 (LH3) enzymes, has been shown to result in the formation of plant-extracted rhCOL1 which forms thermally stable triple helical structures. Though the solvents used to form electrospinning solutions and the electrospinning process itself is believed to remove the native ultrastructure of collagen, this structure can be preserved when the appropriate combination of source material, solvent and spinning parameters are used. For example, D-banding was observed in fibers electrospun from solutions of acid soluble, lyophilized type I collagen from calfskin (83 mg/mL; Sigma-Aldrich, St. Louis, Mo.), dissolved in hexafluoroisopropanol (HFIP) and delivered at a flow rate of 5 mL/hr. Additionally, the use of aqueous solutions such as acetic acid to solubilize the collagen appears to be effective at preserving collagen ultrastructure.

See additional references for collagen-based materials: Blackstone, B. N.; Gallentine, S. C.; Powell, H. M. Collagen-Based Electrospun Materials for Tissue Engineering: A Systematic Review. Bioengineering. 2021, 8, 39; Powell, H. M. and S. T. Boyce. Fiber density of electrospun gelatin scaffolds regulates morphogenesis of dermal-epidermal skin substitutes. J Biomed Mater Res, Volume 84A, Issue 4 Mar. 15, 2008, pages 1078-1086; Britani N. Blackstone, Andre F. Palmer, Horacio R. Rilo, and Heather M. Powell. Scaffold Architecture Controls Insulinoma Clustering, Viability, and Insulin Production. Tissue Engineering Part A, Vol. 20, No. 13-14. Feb. 21, 2014; H. M. Powell et al. Influence of electrospun collagen on wound contraction of engineered skin substitutes. Biomaterials 29 (2008) 834-843; Willard J J, Drexler J W, Das A, Roy S, Shilo S, Shoseyov O, Powell H M. Plant-derived human collagen scaffolds for skin tissue engineering. Tissue Eng Part A. 2013 July; 19(13-14):1507-18.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A biocompatible patch comprising:
a scaffold comprising a plurality of coaxial nanofibers, wherein the nanofibers comprise a polymeric core and a biocompatible shell; and
a cell, a tissue, or an organ in contact with a surface of the scaffold.

2. The biocompatible patch of claim 1, wherein the polymeric core comprises a material selected from the group consisting of polycaprolactone (PCL), poly(lactic-co-glycolic acid) (PLGA), polylactic acid (PLA), polyglycolide (PGA), and polyurethane (PU).

3. The biocompatible patch of claim 1, wherein the biocompatible shell comprises a material selected from the group consisting of gelatin, collagen, collagen type I, collagen type IV, Matrigel, elastin, silk, laminin, and polyvinyl alcohol.

4. The biocompatible patch of claim 3, wherein the biocompatible shell comprises gelatin.

5. The biocompatible patch of claim 3, wherein the biocompatible shell comprises collagen.

6. The biocompatible patch of claim 1, wherein the plurality of nanofibers are aligned.

7. The biocompatible patch of claim 1, wherein the coaxial nanofibers have a diameter between about 200 nm to about 1000 nm.

8. The biocompatible patch of claim 1, wherein the biocompatible patch has a tensile strength between about 0.5 MPa to about 3.0 MPa.

9. The biocompatible patch of claim 1, further comprising a growth factor.

10. The biocompatible patch of claim 9, wherein the growth factor is incorporated into the biocompatible shell or on the surface of the biocompatible shell.

11. The biocompatible patch of claim 9, wherein the growth factor is selected from the group consisting of vascular endothelial growth factor (VEGF), basic fibroblast growth factor (bFGF), insulin-like growth factor (IGF), placental growth factor (PIGF), angiopoietin-1, platelet derived growth factor-BB (PDGF-BB), and transforming growth factor β (TGF-β).

12. The biocompatible patch of claim 11, wherein the growth factor is basic fibroblast growth factor (bFGF).

13. The biocompatible patch of claim 1, further comprising fibronectin on the surface of the biocompatible shell.

14. The biocompatible patch of claim 1, wherein the cell comprises a stem cell or a cardiac cell.

15. The biocompatible patch of claim 14, wherein the stem cell is selected from the group consisting of an induced pluripotent stem cell, a mesenchymal stem cell, and a cardiac progenitor cell.

16. The biocompatible patch of claim 1, wherein the tissue comprises a cardiac tissue.

17. The biocompatible patch of claim 1, wherein the biocompatible patch is coated with polydopamine (PDA).

18. A method for treating a damaged cardiac tissue in a subject, comprising transplanting the biocompatible patch of claim 1 to a site of the damaged cardiac tissue in the subject.

19. The method of claim 18, further comprising culturing the cell and the scaffold of the biocompatible patch ex vivo for at least 10 days prior to transplantation.

20. A method of differentiating a stem cell, comprising:
contacting a stem cell with a surface of the scaffold of the biocompatible patch of claim 1; and
culturing the stem cell.

21. The method of claim 20, wherein the stem cell is selected from the group consisting of an induced pluripotent stem cell, a mesenchymal stem cell, and a cardiac progenitor cell.
